(19) United States
(12) Patent Application Publication
(10) Pub. No.: US 2022/0387310 A1
(43) Pub. Date: Dec. 8, 2022

(54) FORMULATIONS FOR RELEASE-RATE MODULATING FILMS FOR GASTRIC RESIDENCE SYSTEMS

(71) Applicant: Lyndra Therapeutics, Inc., Watertown, MA (US)

(72) Inventors: David ALTREUTER, Wayland, MA (US); Alisha WEIGHT, Cambridge, MA (US); Saumya MOORTHY, Medford, MA (US); Tammy TAI, Cambridge, MA (US); Estelle BEGUIN, Watertown, MA (US)

(21) Appl. No.: 17/774,127

(22) PCT Filed: Nov. 6, 2020
(86) PCT No.: PCT/US2020/059536

§ 371 Date: May 3, 2022

Related U.S. Application Data
(60) Provisional application No. 62/933,313, filed on Nov. 8, 2019.

(51) Int. Cl. A61K 9/00; A61K 47/34

(52) U.S. Cl. CPC A61K 9/0065; A61K 47/34

(57) ABSTRACT

Provided herein are gastric residence systems, or components of gastric residence system such as arms (elongate members) or segments of gastric residence systems, with release rate-modulating films. The release rate-modulating films provide good control over release of agents (such as therapeutic, diagnostic, or nutritional agents) from the gastric residence systems. The release rate-modulating films disclosed herein resist changes to their release properties during heat-assisted assembly of the gastric residence systems.

### CROSS-REFERENCE TO RELATED APPLICATIONS

This application claims priority benefit of U.S. Provisional Patent Application No. 62/933,313 filed Nov. 8, 2019. The entire contents of that application are hereby incorporated by reference herein.

### FIELD OF THE INVENTION

The current disclosure relates to systems which remain in the stomach for extended periods for sustained release of pharmaceuticals, and methods of use thereof; and to release-rate modifying films for use with such systems.

### BACKGROUND OF THE INVENTION

Gastric residence systems are delivery systems for agents which remain in the stomach for days to weeks, or even over longer periods, during which time drugs or other agents can elute from the systems for absorption in the gastrointestinal tract. Examples of such systems are described in International Patent Application Nos. WO 2015/191920, WO 2015/191925, WO 2017/070612, WO 2017/100367, and WO 2017/205844. These systems deliver agents by gradual release over time from carrier polymer-agent blends, so that the system releases an agent or agents over the period of gastric residence. Achieving the desired release rate requires careful selection of the materials for use in the gastric residence system. International Patent Application No. WO 2018/227147 describes selection of materials for release-rate modulating films for gastric residence systems which provide good control over kinetics of release from the systems. The release-rate modulating films can be placed over the agent-containing portion of the gastric residence system in order to control agent release. Use of a release rate-modulating polymer film as a coating over the carrier polymer-agent blend provides several significant advantages. Release rate-modulating polymer films reduce the burst release of agent upon initial contact with gastric fluid and improve the linearity of agent release over the residence, which provides better regulation of dosing from the gastric residence systems. Some compositions of release rate-modulating polymer films can also significantly reduce burst release upon exposure to alcohol, as compared to systems lacking such films.

Release-rate modulating films are relatively thin, and when a film-coated carrier polymer-agent blend component is assembled into a gastric residence system, the film can be subject to disruption. Assembly of gastric residence systems can be done using heat-assisted assembly, and it is particularly important to prevent disruptions in the release-rate modulating properties of the film during such heat-assisted assembly. The current disclosure provides improved release rate-modulating films for use in gastric residence systems which resist such disruption during heat-assisted assembly.

### SUMMARY OF THE INVENTION

Gastric residence systems are generally made from several different components. Examples of such components can include the elongate

members or “arms” of gastric residence systems, such as the arms of star-shaped (stellate) gastric residence systems. The arms can comprise a carrier polymer, an agent (e.g., a drug), and various excipients. A release-rate modulating film can then be placed over the arms for control of the kinetics of release. Other components of a gastric residence system can include one or more elastomeric components, such as a central elastomer; and linkers or disintegrating matrices which connect the various components. Connecting the various components is often performed by heating at least one of the components which is to be connected to the other components, and sometimes by heating all of the components which are being connected. Heating can be accomplished by contact with a heated platen, by using an infrared radiation source, by using an infrared laser, or by using other heat-producing, heat-emitting, or heat-transferring devices. The various components of the gastric residence system should be resistant to changes in their properties during such heat-assisted assembly.

Release-rate modulating films are relatively thin. If an agent-containing portion (such as an arm of a star-shaped system) of a gastric residence system bears a release-rate modulating film, it is particularly important to prevent disruptions in the release-rate modulating properties of the film during heat-assisted assembly. The current disclosure provides improved release rate-modulating films for use in gastric residence systems. One aspect of the improved release rate-modulating films is increased resistance to disruption during heat-assisted assembly, so that the release properties of the agent from the agent-containing components of the system after assembly are substantially the same as the release properties of the agent from the agent-containing components of the system prior to assembly.

The disclosure further comprises methods of administering a gastric residence system to a patient, comprising administering a container containing any embodiment of the gastric residence systems disclosed herein in a compacted state to a patient, wherein the container enters the stomach of the patient and dissolves after entry into the stomach, releasing the gastric residence system which then adopts its uncompacted state. Preferably, the patient is a human. The container containing the gastric residence system can be administered by swallowing, by feeding tube, or by gastrostomy tube.

The invention provides gastric residence systems which have segments covered with release rate-modulating polymer films. The invention also provides arms covered with release rate-modulating polymer films suitable for use in gastric residence systems. The invention also provides arms of gastric residence systems which have segments covered with release rate-modulating polymer films. The invention also provides segments covered with release rate-modulating polymer films suitable for use in gastric residence systems. Methods of making the segments, arms, and gastric residence systems are also provided. Methods of using the gastric residence systems are also provided.

In some embodiments, the invention provides arms for use in a gastric residence system, comprising: a carrier polymer, at least one agent or a pharmaceutically acceptable salt thereof, and a release rate-modulating film coated on at least a portion of the surface of the arm; wherein the release rate-modulating film comprises poly-D,L-lactide (PDL) and poly-D,L-lactide/glycolide (PDLG). In some embodiments, the PDL comprises PDL having an intrinsic viscosity of about 1 dl/g to about 5 dl/g; of about 1 dl/g to about 4 dl/g; or of about 1.6 dl/g to about 2.4 dl/g. In some embodiments, the PDLG comprises PDLG having an intrinsic viscosity of about 0.1 dl/g to about 3 dl/g; of about 0.1 dl/g to about 1.5 dl/g; or of about 0.1 dl/g to about 0.5 dl/g. In some embodiments according to any one of the arms disclosed herein, the PDL:PDLG ratio is between about 2:1 to about 1:2 (weight/weight). In some embodiments, PDL:PDLG ratio is between about 1.25:1 to about 1:1.25 (w/w). In some embodiments, the PDL:PDLG ratio is about 1:1 (w/w). In some embodiments, the release rate-modulating film is substantially free of porogen. In some embodiments according to any one of the arms disclosed herein, the increase in the weight of the arm due to addition of the release rate-modulating film is about 2% to about 6% of the weight of the uncoated arm. In some embodiments, the release rate of agent from the arm in aqueous media is substantially linear over at least a 96-hour period. In some embodiments, the release rate of agent from the arm is substantially the same before and after thermal cycling. In some embodiments, the invention provides a gastric residence system comprising any one of the arms disclosed herein. In some embodiments, the invention provides a gastric residence system comprising one or more of any of the arms disclosed herein and a central elastic polymeric component; wherein the one or more arms are each connected to the central elastic polymeric component via a separate linker component; wherein the gastric residence system is configured to be folded and physically constrained during administration and is configured to assume an open retention shape upon removal of a constraint; wherein change between the folded shape and the open retention shape is mediated by the elastic polymeric component that undergoes elastic deformation when the residence system is in the folded shape and recoils when the gastric residence system assumes the open retention shape; and wherein said linker degrades, dissolves, disassociates, or mechanically weakens in a gastric environment which results in loss of retention shape integrity and passage out of a gastric cavity. In some embodiments the release rate-modulating film is applied by pan coating. In some embodiments, the release rate-modulating film is applied by dip coating. In some embodiments, the at least one agent or a pharmaceutically acceptable salt thereof comprises memantine. In some embodiments, the at least one agent or a pharmaceutically acceptable salt thereof comprises donepezil. In some embodiments, the at least one agent or a pharmaceutically acceptable salt thereof comprises memantine and donepezil. In some embodiments, the at least one agent or a pharmaceutically acceptable salt thereof comprises risperidone. In some embodiments, the at least one agent or a pharmaceutically acceptable salt thereof comprises dapagliflozin.

In some embodiments, the invention provides arms for use in a gastric residence system, comprising: a carrier polymer, at least one agent or a pharmaceutically acceptable salt thereof, and a release rate-modulating film coated on at least a portion of the surface of the arm; wherein the release rate-modulating film comprises high molecular weight polycaprolactone (PCL-HMW) and low molecular weight polycaprolactone (PCL-LMW). In some embodiments, the PCL-HMW comprises PCL of about Mₙ 75,000 to about Mₙ 250,000; or PCL having an intrinsic viscosity of about 1.0 dl/g to about 2.4 dl/g, about 1.2 dl/g to about 2.4 dl/g, or about 1.6 dl/g to about 2.4 dl/g. In some embodiments, the PCL-LMW comprises PCL of about Mₙ 10,000 to about Mₙ 20,000; or PCL having an intrinsic viscosity of about 0.1 dl/g to about 0.8 dl/g. In some embodiments, the PCL-HMW comprises PCL of about Mₙ 75,000 to about Mₙ 250,000, or PCL having an intrinsic viscosity of about 1.0 dl/g to about 2.4 dl/g, about 1.2 dl/g to about 2.4 dl/g, or about 1.6 dl/g to about 2.4 dl/g; and the PCL-LMW comprises PCL of about Mₙ 10,000 to about Mₙ 20,000, or PCL having an intrinsic viscosity of about 0.1 dl/g to about 0.8 dl/g. In some embodiments according to any one of the arms disclosed herein, the (PCL-HMW):(PCL-LMW) ratio is between about 1:4 to about 95:5 (weight/weight). In some embodiments, the (PCL-HMW):(PCL-LMW) ratio is between about 2:3 to about 95:5 (weight/weight). In some embodiments, the (PCL-HMW):(PCL-LMW) ratio is between about 3:1 to about 95:5 (weight/weight). In some embodiments, the (PCL-HMW):(PCL-LMW) ratio is about 9:1 (w/w). In some embodiments, the release rate-modulating film is substantially free of porogen. In some embodiments according to any one of the arms disclosed herein, the increase in the weight of the arm due to addition of the release rate-modulating film is about 2% to about 6% of the weight of the uncoated arm. In some embodiments, the release rate of agent from the arm in aqueous media is substantially linear over at least a 96-hour period. In some embodiments, the release rate of agent from the arm is substantially the same before and after thermal cycling. In some embodiments, the invention provides a gastric residence system comprising any one of the arms disclosed herein. In some embodiments, the invention provides a gastric residence system comprising one or more of any of the arms disclosed herein and a central elastic polymeric component; wherein the one or more arms are each connected to the central elastic polymeric component via a separate linker component; wherein the gastric residence system is configured to be folded and physically constrained during administration and is configured to assume an open retention shape upon removal of a constraint; wherein change between the folded shape and the open retention shape is mediated by the elastic polymeric component that undergoes elastic deformation when the residence system is in the folded shape and recoils when the gastric residence system assumes the open retention shape; and wherein said linker degrades, dissolves, disassociates, or mechanically weakens in a gastric environment which results in loss of retention shape integrity and passage out of a gastric cavity. In some embodiments the release rate-modulating film is applied by pan coating. In some embodiments, the release rate-modulating film is applied by dip coating. In some embodiments, the at least one agent or a pharmaceutically acceptable salt thereof comprises memantine. In some embodiments, the at least one agent or a pharmaceutically acceptable salt thereof comprises donepezil. In some embodiments, the at least one agent or a pharmaceutically acceptable salt thereof comprises memantine and donepezil. In some embodiments, the at least one agent or a pharmaceutically acceptable salt thereof comprises risperidone. In some embodiments, the at least one agent or a pharmaceutically acceptable salt thereof comprises dapagliflozin.

In some embodiments, the invention provides arms for use in a gastric residence system, comprising: a carrier polymer, at least one agent or a pharmaceutically acceptable salt thereof, and a release rate-modulating film coated on at least a portion of the surface of the arm; wherein the release rate-modulating film comprises poly-D,L-lactide (PDL). In some embodiments, the PDL comprises PDL having an intrinsic viscosity of about 1 dl/g to about 5 dl/g; or of about 1 dl/g to about 4 dl/g. In some embodiments, the PDL comprises PDL having an intrinsic viscosity of about 1.6 dl/g to about 2.4 dl/g. In some embodiments, the release rate-modulating film further comprises polycaprolactone (PCL) and polyethylene glycol (PEG). In some embodiments, the PCL comprises PCL of about Mₙ 75,000 to about Mₙ 250,000. In some embodiments, the PEG comprises PEG of about Mₙ 800 to about Mₙ 20,000. In some embodiments according to any one of the arms disclosed herein, the PDL comprises between about 15% to about 80% of the release rate-modulating film, the PCL comprises between about 15% to about 75% of the release rate-modulating film, and the PEG comprises between about 5% to about 15% of the release rate-modulating film, by weight. In some embodiments, the PDL:PCL:PEG ratio is about 9:27:4 (w/w/w). In some embodiments, the PDL:PCL:PEG ratio is about 36:9:5 (w/w/w). In some embodiments, the release rate-modulating film is substantially free of porogen. In some embodiments according to any one of the arms disclosed herein, the increase in the weight of the arm due to addition of the release rate-modulating film is about 2% to about 6% of the weight of the uncoated arm. In some embodiments, the release rate of agent from the arm in aqueous media is substantially linear over at least a 96-hour period. In some embodiments, the release rate of agent from the arm is substantially the same before and after thermal cycling. In some embodiments, the invention provides a gastric residence system comprising any one of the arms disclosed herein. In some embodiments, the invention provides a gastric residence system comprising any one of the arms disclosed herein and a central elastic polymeric component; wherein the one or more arms are each connected to the central elastic polymeric component via a separate linker component; wherein the gastric residence system is configured to be folded and physically constrained during administration and is configured to assume an open retention shape upon removal of a constraint; wherein change between the folded shape and the open retention shape is mediated by the elastic polymeric component that undergoes elastic deformation when the residence system is in the folded shape and recoils when the gastric residence system assumes the open retention shape; and wherein said linker degrades, dissolves, disassociates, or mechanically weakens in a gastric environment which results in loss of retention shape integrity and passage out of a gastric cavity.

In some embodiments according to any one of the arms described herein, the release rate-modulating film further comprises a polyethylene glycol-polypropylene glycol-polyethylene glycol (PEG-PPG-PEG) block copolymer. In some embodiments, the PEG-PPG-PEG block copolymer comprises PEG-PPG-PEG block copolymer of Mₙ about 14,000 to about 15,000. In some embodiments, the PEG-PPG-PEG block copolymer comprises about 75% to about 90% ethylene glycol. In some embodiments, the (PDL):(PEG-PPG-PEG block copolymer) ratio is between about 85:15 to about 95:5 (w/w). In some embodiments, the (PDL):(PEG-PPG-PEG block copolymer) ratio is about 9:1 (w/w). In some embodiments, the release rate-modulating film is substantially free of porogen. In some embodiments according to any one of the arms described herein, the increase in the weight of the arm due to addition of the release rate-modulating film is about 2% to about 6% of the weight of the uncoated arm. In some embodiments, the release rate of agent from the arm in aqueous media is substantially linear over at least a 96-hour period. In some embodiments, the release rate of agent from the arm is substantially the same before and after thermal cycling. In some embodiments, the invention provides a gastric residence system comprising any one of the arms disclosed herein. In some embodiments, the invention provides a gastric residence system comprising one or more of any of the arms disclosed herein and a central elastic polymeric component; wherein the one or more arms are each connected to the central elastic polymeric component via a separate linker component; wherein the gastric residence system is configured to be folded and physically constrained during administration and is configured to assume an open retention shape upon removal of a constraint; wherein change between the folded shape and the open retention shape is mediated by the elastic polymeric component that undergoes elastic deformation when the residence system is in the folded shape and recoils when the gastric residence system assumes the open retention shape; and wherein said linker degrades, dissolves, disassociates, or mechanically weakens in a gastric environment which results in loss of retention shape integrity and passage out of a gastric cavity. In some embodiments the release rate-modulating film is applied by pan coating. In some embodiments, the release rate-modulating film is applied by dip coating. In some embodiments, the at least one agent or a pharmaceutically acceptable salt thereof comprises memantine. In some embodiments, the at least one agent or a pharmaceutically acceptable salt thereof comprises donepezil. In some embodiments, the at least one agent or a pharmaceutically acceptable salt thereof comprises memantine and donepezil. In some embodiments, the at least one agent or a pharmaceutically acceptable salt thereof comprises risperidone. In some embodiments, the at least one agent or a pharmaceutically acceptable salt thereof comprises dapagliflozin.

In some embodiments according to any one of the arms described herein, the release rate-modulating film further comprises polyethylene glycol (PEG). In some embodiments according to any one of the arms described herein, the release rate-modulating film further comprises polypropylene glycol (PPG). In some embodiments according to any one of the arms described herein, the release rate-modulating film further comprises polyethylene glycol (PEG) and polypropylene glycol (PPG). In some embodiments, the PDL comprises between about 75% to about 95% of the release rate-modulating film, the PEG comprises between about 3% to about 10% of the release rate-modulating film, and the PPG comprises between about 1% to about 7% of the release rate-modulating film, by weight. In some embodiments, the (PDL):(PEG):(PPG) ratio is about 90:(six and two-thirds):(three and one-third) by weight. In some embodiments, the PEG comprises PEG of molecular weight about 800 to about 1,200. In some embodiments, the PPG comprises PPG of at least about Mₙ 2,500. In some embodiments, the PPG comprises PPG of about Mₙ 2,500 to about Mₙ 6,000. In some embodiments, the release rate-modulating film is substantially free of porogen. In some embodiments according to any one of the arms described herein, the increase in the weight of the arm due to addition of the release rate-modulating film is about 2% to about 6% of the weight of the uncoated arm. In some embodiments, the release rate of agent from the arm in aqueous media is substantially linear over at least a 96-hour period. In some embodiments, the release rate of agent from the arm is substantially the same before and after thermal cycling. In some embodiments, the invention provides a gastric residence system comprising any one of the arms disclosed herein. In some embodiments, the invention provides a gastric residence system comprising one or more of any of the arms disclosed herein and a central elastic polymeric component; wherein the one or more arms are each connected to the central elastic polymeric component via a separate linker component; wherein the gastric residence system is configured to be folded and physically constrained during administration and is configured to assume an open retention shape upon removal of a constraint; wherein change between the folded shape and the open retention shape is mediated by the elastic polymeric component that undergoes elastic deformation when the residence system is in the folded shape and recoils when the gastric residence system assumes the open retention shape; and wherein said linker degrades, dissolves, disassociates, or mechanically weakens in a gastric environment which results in loss of retention shape integrity and passage out of a gastric cavity. In some embodiments the release rate-modulating film is applied by pan coating. In some embodiments, the release rate-modulating film is applied by dip coating. In some embodiments, the at least one agent or a pharmaceutically acceptable salt thereof comprises memantine. In some embodiments, the at least one agent or a pharmaceutically acceptable salt thereof comprises donepezil. In some embodiments, the at least one agent or a pharmaceutically acceptable salt thereof comprises memantine and donepezil. In some embodiments, the at least one agent or a pharmaceutically acceptable salt thereof comprises risperidone. In some embodiments, the at least one agent or a pharmaceutically acceptable salt thereof comprises dapagliflozin.

In some embodiments, the invention provides arms for use in a gastric residence system, comprising: a carrier polymer, at least one agent or a pharmaceutically acceptable salt thereof, and a release rate-modulating film coated on at least a portion of the surface of the arm; wherein the release rate-modulating film comprises poly-D-lactide-polycaprolactone co-polymer (PDL-PCL copolymer). In some embodiments, PDL comprises between about 15% to about 90% of the PDL-PCL copolymer. In some embodiments, PDL comprises between about 15% to about 35% of the PDL-PCL copolymer. In some embodiments, PDL comprises between about 70% to about 90% of the PDL-PCL copolymer. In some embodiments, the PDL-PCL copolymer comprises PDL-PCL copolymer having intrinsic viscosity of about 0.6 dl/g to about 4 dl/g, of about 0.6 dl/g to about 2 dl/g, or of about 0.6 dl/g to about 1 dl/g. In some embodiments according to any one of the arms described herein, the release rate-modulating film further comprises PEG. In some embodiments, the PEG comprises PEG of average molecular weight between about 800 and about 1,200. In some embodiments, the PDL-PCL copolymer comprises about 75% to about 95% of the release rate modulating film by weight and the PEG comprises about 5% to about 25% of the release rate modulating film by weight. In some embodiments, the PDL-PCL copolymer comprises about 90% of the release rate modulating film by weight and the PEG comprises about 10% of the release rate modulating film by weight. In some embodiments, the release rate-modulating film is substantially free of porogen. In some embodiments according to any one of the arms described herein, the increase in the weight of the arm due to addition of the release rate-modulating film is about 2% to about 6% of the weight of the uncoated arm. In some embodiments, the release rate of agent from the arm in aqueous media is substantially linear over at least a 96-hour period. In some embodiments, the release rate of agent from the arm is substantially the same before and after thermal cycling. In some embodiments, the invention provides a gastric residence system comprising any one of the arms disclosed herein. In some embodiments, the invention provides a gastric residence system comprising one or more of any of the arms disclosed herein and a central elastic polymeric component; wherein the one or more arms are each connected to the central elastic polymeric component via a separate linker component; wherein the gastric residence system is configured to be folded and physically constrained during administration and is configured to assume an open retention shape upon removal of a constraint; wherein change between the folded shape and the open retention shape is mediated by the elastic polymeric component that undergoes elastic deformation when the residence system is in the folded shape and recoils when the gastric residence system assumes the open retention shape; and wherein said linker degrades, dissolves, disassociates, or mechanically weakens in a gastric environment which results in loss of retention shape integrity and passage out of a gastric cavity. In some embodiments the release rate-modulating film is applied by pan coating. In some embodiments, the release rate-modulating film is applied by dip coating. In some embodiments, the at least one agent or a pharmaceutically acceptable salt thereof comprises memantine. In some embodiments, the at least one agent or a pharmaceutically acceptable salt thereof comprises donepezil. In some embodiments, the at least one agent or a pharmaceutically acceptable salt thereof comprises memantine and donepezil. In some embodiments, the at least one agent or a pharmaceutically acceptable salt thereof comprises risperidone. In some embodiments, the at least one agent or a pharmaceutically acceptable salt thereof comprises dapagliflozin.

In some embodiments according to any one of the arms or gastric residence systems described herein, the release rate-modulating film is applied by pan coating. In some embodiments according to any one of the arms or gastric residence systems described herein, the release rate-modulating film is applied by dip coating.

In some embodiments according to any one of the arms or gastric residence systems described herein, the at least one agent or a pharmaceutically acceptable salt thereof comprises one or more of drug, a pro-drug, a biologic, a statin, rosuvastatin, a nonsteroidal anti-inflammatory drug (NSAID), meloxicam, a selective serotonin reuptake inhibitor (SSRs), escitalopram, citalopram, a blood thinner, clopidogrel, a steroid, prednisone, an antipsychotic, aripiprazole, risperidone, an analgesic, buprenorphine, an opioid antagonist, naloxone, an anti-asthmatic, montelukast, an anti-dementia drug, memantine, a cardiac glycoside, digoxin, an alpha blocker, tamsulosin, a cholesterol absorption inhibitor, ezetimibe, an anti-gout treatment, colchicine, an antihistamine, loratadine, cetirizine, an opioid, loperamide, a proton-pump inhibitor, omeprazole, an antiviral agent, entecavir, an antibiotic, doxycycline, ciprofloxacin, azithromycin, an anti-malarial agent, levothyroxine, a substance abuse treatment, methadone, varenicline, a contraceptive, a stimulant, caffeine, a nutrient, folic acid, calcium, iodine, iron, zinc, thiamine, niacin, vitamin C, vitamin D, biotin, a plant extract, a phytohormone, a vitamin, a mineral, a protein, a polypeptide, a polynucleotide, a hormone, an anti-inflammatory drug, an antipyretic, an antidepressant, an antiepileptic, an antipsychotic agent, a neuroprotective agent, an anti-proliferative, an anti-cancer agent, an antimigraine drug, a prostaglandin, an antimicrobial, an antifungals, an antiparasitic, an anti-muscarinic, an anxiolytic, a bacteriostatic, an immunosuppressant agent, a sedative, a hypnotic, a bronchodilator, a cardiovascular drug, an anesthetic, an anti-coagulant, an enzyme inhibitor, a corticosteroid, a dopaminergic, an electrolyte, a gastro-intestinal drug, a muscle relaxant, a parasympathomimetic, an anorectic, an anti-narcoleptics, quinine, lumefantrine, chloroquine, amodiaquine, pyrimethamine, proguanil, chlorproguanil-dapsone, a sulfonamide, sulfadoxine, sulfamethoxypyridazine, mefloquine, atovaquone, primaquine, halofantrine, doxycycline, clindamycin, artemisinin, an artemisinin derivative, artemether, dihydroartemisinin, arteether, or artesunate. In some embodiments, the at least one agent or a pharmaceutically acceptable salt thereof comprises memantine. In some embodiments, the at least one agent or a pharmaceutically acceptable salt thereof comprises donepezil. In some embodiments, the at least one agent or a pharmaceutically acceptable salt thereof comprises memantine and donepezil. In some embodiments, the at least one agent or a pharmaceutically acceptable salt thereof comprises risperidone. In some embodiments, the at least one agent or a pharmaceutically acceptable salt thereof comprises dapagliflozin.

In any of the embodiments of the release rate-modulating polymer films, or any of the embodiments of segments covered with a release rate-modulating polymer film, the release rate-modulating polymer film do not contain agents; that is, the films do not contain any substance intended for therapeutic, diagnostic, or nutritional use.

In any of the embodiments of the release rate-modulating polymer films, or any of the embodiments of segments covered with a release rate-modulating polymer film, the release-rate modulating polymer film does not add substantially to the strength of the carrier polymer-agent segment that it covers.

It is contemplated that any features from any embodiment disclosed herein can be combined with any features from any other embodiment disclosed herein where possible. In this fashion, hybrid configurations of the disclosed features are within the scope of the present disclosure.

### BRIEF DESCRIPTION OF THE DRAWINGS

FIG. 1 shows drug release curves for donepezil (DNP) and memantine (MEM) from drug-loaded arms before and after exposure to welding conditions.

FIG. 2 shows drug release curves for donepezil from donepezil-loaded arms (DN34) before and after exposure to welding conditions.

FIG. 3 shows drug release curves for donepezil from donepezil-loaded arms (DN34) before and after exposure to welding conditions.

FIG. 4 shows drug release curves for memantine from memantine-loaded arms (M116) before and after exposure to welding conditions.

FIG. 5 shows drug release curves for memantine from memantine-loaded arms (M122) before and after exposure to welding conditions.

FIG. 6 shows drug release curves for memantine from memantine-loaded arms (M122) before and after exposure to welding conditions.

FIG. 7 shows drug release curves for donepezil (DNP) and memantine (MEM) from drug-loaded arms before and after exposure to welding conditions.

FIG. 8A shows drug release curves for memantine (MEM) from drug-loaded arms before and after exposure to welding conditions.

FIG. 8B shows drug release curves for donepezil (DNP) from drug-loaded arms before and after exposure to welding conditions.

FIG. 9 shows drug release curves for memantine from drug-loaded arms before and after exposure to welding conditions, at different coat weights.

FIG. 10 shows drug release curves for dapagliflozin (DAPA) from coated and uncoated drug-loaded arms before and after exposure to welding conditions, with IR exposure to 4 mm out of 10 mm of the drug-loaded arm.

FIG. 11 shows drug release curves for dapagliflozin (DAPA) from coated drug-loaded arms before and after exposure to welding conditions, with IR exposure to 15 mm out of 15 mm of the drug-loaded arm.

FIG. 12 shows drug release curves for dapagliflozin (DAPA) from coated drug-loaded arms before and after welding, where inactive segments are welded to either end of the drug-loaded arm, with IR exposure to 15 mm out of 15 mm of the arm, including 4 mm out of 4 mm of the drug-containing arm segment.

FIG. 13 shows an exemplary method of bonding components together to form a gastric residence system.

FIG. 14A shows a stellate design of a gastric residence system in its uncompacted state.

FIG. 14B shows a stellate design of a gastric residence system in a compacted or folded state.

FIG. 14C shows a ring design of a gastric residence system in an uncompacted state.

### DETAILED DESCRIPTION OF THE INVENTION

### Definitions

A “carrier polymer” is a polymer suitable for blending with an agent, such as a drug, for use in a gastric residence system.

An “agent” is any substance intended for therapeutic, diagnostic, or nutritional use in a patient, individual, or subject. Agents include, but are not limited to, drugs, nutrients, vitamins, and minerals.

A “dispersant” is defined as a substance which aids in the minimization of particle size of agent and the dispersal of agent particles in the carrier polymer matrix. That is, the dispersant helps minimize or prevent aggregation or flocculation of particles during fabrication of the systems. Thus, the dispersant has anti-aggregant activity and anti-flocculant activity, and helps maintain an even distribution of agent particles in the carrier polymer matrix.

An “excipient” is any substance added to a formulation of an agent that is not the agent itself. Excipients include, but are not limited to, binders, coatings, diluents, disintegrants, emulsifiers, flavorings, glidants, lubricants, and preservatives. The specific category of dispersant falls within the more general category of excipient.

An “elastic polymer” or “elastomer” is a polymer that is capable of being deformed by an applied force from its original shape for a period of time, and which then substantially returns to its original shape once the applied force is removed.

“Approximately constant plasma level” refers to a plasma level that remains within a factor of two of the average plasma level (that is, between 50% and 200% of the average plasma level) measured over the period that the gastric residence system is resident in the stomach.

“Substantially constant plasma level” refers to a plasma level that remains within plus-or-minus 25% of the average plasma level measured over the period that the gastric residence system is resident in the stomach.

“Biocompatible,” when used to describe a material or system, indicates that the material or system does not provoke an adverse reaction, or causes only minimal, tolerable adverse reactions, when in contact with an organism, such as a human. In the context of the gastric residence systems, biocompatibility is assessed in the environment of the gastrointestinal tract.

A “patient,” “individual,” or “subject” refers to a mammal, preferably a human or a domestic animal such as a dog or cat. In a most preferred embodiment, a patient, individual, or subject is a human.

“Treating” a disease or disorder with the systems and methods disclosed herein is defined as administering one or more of the systems disclosed herein to a patient in need thereof, with or without additional agents, in order to reduce or eliminate either the disease or disorder, or one or more symptoms of the disease or disorder, or to retard the progression of the disease or disorder or of one or more symptoms of the disease or disorder, or to reduce the severity of the disease or disorder or of one or more symptoms of the disease or disorder. “Suppression” of a disease or disorder with the systems and methods disclosed herein is defined as administering one or more of the systems disclosed herein to a patient in need thereof, with or without additional agents, in order to inhibit the clinical manifestation of the disease or disorder, or to inhibit the manifestation of adverse symptoms of the disease or disorder. The distinction between treatment and suppression is that treatment occurs after adverse symptoms of the disease or disorder are manifest in a patient, while suppression occurs before adverse symptoms of the disease or disorder are manifest in a patient. Suppression may be partial, substantially total, or total. Because some diseases or disorders are inherited, genetic screening can be used to identify patients at risk of the disease or disorder. The systems and methods disclosed herein can then be used to treat asymptomatic patients at risk of developing the clinical symptoms of the disease or disorder, in order to suppress the appearance of any adverse symptoms.

“Therapeutic use” of the systems disclosed herein is defined as using one or more of the systems disclosed herein to treat a disease or disorder, as defined above. A “therapeutically effective amount” of a therapeutic agent, such as a drug, is an amount of the agent, which, when administered to a patient, is sufficient to reduce or eliminate either a disease or disorder or one or more symptoms of a disease or disorder, or to retard the progression of a disease or disorder or of one or more symptoms of a disease or disorder, or to reduce the severity of a disease or disorder or of one or more symptoms of a disease or disorder. A therapeutically effective amount can be administered to a patient as a single dose, or can be divided and administered as multiple doses.

“Prophylactic use” of the systems disclosed herein is defined as using one or more of the systems disclosed herein to suppress a disease or disorder, as defined above. A “prophylactically effective amount” of an agent is an amount of the agent, which, when administered to a patient, is sufficient to suppress the clinical manifestation of a disease or disorder, or to suppress the manifestation of adverse symptoms of a disease or disorder. A prophylactically effective amount can be administered to a patient as a single dose, or can be divided and administered as multiple doses.

“Therapeutic use” of the systems disclosed herein is defined as using one or more of the systems disclosed herein to treat a disease or disorder, as defined above. A “therapeutically effective amount” of a therapeutic agent, such as a drug, is an amount of the agent, which, when administered to a patient, is sufficient to reduce or eliminate either a disease or disorder or one or more symptoms of a disease or disorder, or to retard the progression of a disease or disorder or of one or more symptoms of a disease or disorder, or to reduce the severity of a disease or disorder or of one or more symptoms of a disease or disorder. A therapeutically effective amount can be administered to a patient as a single dose, or can be divided and administered as multiple doses.

“Prophylactic use” of the systems disclosed herein is defined as using one or more of the systems disclosed herein to suppress a disease or disorder, as defined above. A “prophylactically effective amount” of an agent is an amount of the agent, which, when administered to a patient, is sufficient to suppress the clinical manifestation of a disease or disorder, or to suppress the manifestation of adverse symptoms of a disease or disorder. A prophylactically effective amount can be administered to a patient as a single dose, or can be divided and administered as multiple doses.

As used herein, the singular forms “a”, “an”, and “the” include plural references unless indicated otherwise or the context clearly dictates otherwise.

When numerical values are expressed herein using the term “about” or the term “approximately,” it is understood that both the value specified, as well as values reasonably close to the value specified, are included. For example, the description “about 50° C.” or “approximately 50° C.” includes both the disclosure of 50° C. itself, as well as values close to 50° C. Thus, the phrases “about X” or “approximately X” include a description of the value X itself. If a range is indicated, such as “approximately 50° C. to 60° C.” or “about 50° C. to 60° C.,” it is understood that both the values specified by the endpoints are included, and that values close to each endpoint or both endpoints are included for each endpoint or both endpoints; that is, “approximately 50° C. to 60° C.” (or “about 50° C. to 60° C.”) is equivalent to reciting both “50° C. to 60° C.” and “approximately 50° C. to approximately 60° C.” (or “about 50° C. to 60° C.”).

With respect to numerical ranges disclosed in the present description, any disclosed upper limit for a component may be combined with any disclosed lower limit for that component to provide a range (provided that the upper limit is greater than the lower limit with which it is to be combined). Each of these combinations of disclosed upper and lower limits are explicitly envisaged herein. For example, if ranges for the amount of a particular component are given as 10% to 30%, 10% to 12%, and 15% to 20%, the ranges 10% to 20% and 15% to 30% are also envisaged, whereas the combination of a 15% lower limit and a 12% upper limit is not possible and hence is not envisaged.

Unless otherwise specified, percentages of ingredients in compositions are expressed as weight percent, or weight/weight percent. It is understood that reference to relative weight percentages in a composition assumes that the combined total weight percentages of all components in the composition add up to 100. It is further understood that relative weight percentages of one or more components may be adjusted upwards or downwards such that the weight percent of the components in the composition combine to a total of 100, provided that the weight percent of any particular component does not fall outside the limits of the range specified for that component.

Some embodiments described herein are recited as “comprising” or “comprises” with respect to their various elements. In alternative embodiments, those elements can be recited with the transitional phrase “consisting essentially of” or “consists essentially of” as applied to those elements. In further alternative embodiments, those elements can be recited with the transitional phrase “consisting of” or “consists of” as applied to those elements. Thus, for example, if a composition or method is disclosed herein as comprising A and B, the alternative embodiment for that composition or method of “consisting essentially of A and B” and the alternative embodiment for that composition or method of “consisting of A and B” are also considered to have been disclosed herein. Likewise, embodiments recited as “consisting essentially of” or “consisting of” with respect to their various elements can also be recited as “comprising” as applied to those elements. Finally, embodiments recited as “consisting essentially of” with respect to their various elements can also be recited as “consisting of” as applied to those elements, and embodiments recited as “consisting of” with respect to their various elements can also be recited as “consisting essentially of” as applied to those elements.

When a composition or system is described as “consisting essentially of” the listed elements, the composition or system contains the elements expressly listed, and may contain other elements which do not materially affect the condition being treated (for compositions for treating conditions), or the properties of the described system (for compositions comprising a system). However, the composition or system either does not contain any other elements which do materially affect the condition being treated other than those elements expressly listed (for compositions for treating systems) or does not contain any other elements which do materially affect the properties of the system (for compositions comprising a system); or, if the composition or system does contain extra elements other than those listed which may materially affect the condition being treated or the properties of the system, the composition or system does not contain a sufficient concentration or amount of those extra elements to materially affect the condition being treated or the properties of the system. When a method is described as “consisting essentially of” the listed steps, the method contains the steps listed, and may contain other steps that do not materially affect the condition being treated by the method or the properties of the system produced by the method, but the method does not contain any other steps which materially affect the condition being treated or the system produced other than those steps expressly listed.

This disclosure provides several embodiments. It is contemplated that any features from any embodiment can be combined with any features from any other embodiment where possible. In this fashion, hybrid configurations of the disclosed features are within the scope of the present disclosure.

In addition to the embodiments and methods disclosed here, additional embodiments of gastric residence systems, and methods of making and using such systems, are disclosed in International Patent Application Nos. WO 2015/191920, WO 2015/191925, WO 2017/070612, WO 2017/100367, and WO 2017/205844, which are incorporated by reference herein in their entirety.

The following abbreviations for polymers are used:

| Abbreviation | Polymer |
|---|---|
| PDL | poly(DL-lactide); inherent viscosity 1.6-2.4 dl/g |
| | (CHCl₃), Tₘ 165-180° C. |
| PCL HMW | polycaprolactone; MW (ave) 200,000 |
| PCL LMW | polycaprolactone; MW (ave) 15,000 |
| VA64 | copovidone; Tₘ 140° C., T_{g} 101° C. |
| K90F | povidone; T_{g} 156° C. |
| PEG1 | polyethylene glycol; MW (ave) 1,000 |
| L-31 | Pluronic ® L-31; PEG-PPG-PEG block co-polymer; |
| | MW (ave) 1,100 (Mₙ) |
| PPG | polypropylene glycol |
| PDLG | copolymer of DL-lactide and glycolide); inherent |
| | viscosity 1.6-2.4 dl/g (CHCl₃) |
| PCL triol | polycaprolactone triol; MW (ave) 900 (Mₙ) |
| F-108 | Pluronic ® F-108; PEG-PPG-PEG block co-polymer |
| PDL-PCL 25-75 | poly-D-lactide-polycaprolactone co-polymer |
| PDL-PCL 80-20 | poly-D-lactide-polycaprolactone co-polymer |
| PG | propylene glycol |
| PVPP | crospovidone |
| PVAc | polyvinylacetate |
| PEG10 | polyethylene glycol; MW (ave) 10,000 |

PLURONIC® is a registered trademark of BASF Corporation for polyoxyalkylene ethers.

### Release Rate-Modulating Polymer Films

The current disclosure provides release-rate modulating polymer films which can be coated onto components of gastric residence systems which release agents, such as drugs. Components coated with the release-rate modulating polymer films disclosed herein have substantially the same release-rate properties before and after exposure to heat which occurs during heat-assisted assembly of a gastric residence system. The current disclosure also provides, inter alia, gastric residence systems, arms (elongate members) of gastric residence systems, and segments for use in gastric residence systems and arms of gastric residence systems, which are coated with such release rate-modulating films.

In some embodiments, the release rate modulating film of any of the gastric residence systems disclosed herein does not cover the enteric linkers, time-dependent linkers, disintegrating matrices, or other linkers of the gastric residence system. If a release-rate modulating polymer film is coated on the surface of an arm which comprises one or more linkers, such as a coupling polymer, enteric polymer, enteric linker, time-dependent linker, disintegrating polymer, disintegrating matrix, or other linker, the film does not cover or coat the linkers. This is readily accomplished by applying a release rate-modulating film to segments which will comprise an arm, and then linking the coated segments together with linkers or disintegrating matrices to form an arm; the segments comprising carrier polymer-agent (or agent salt) will thus be coated with the release rate-modulating film, but the linkers or disintegrating matrices will not be coated with the release rate-modulating film.

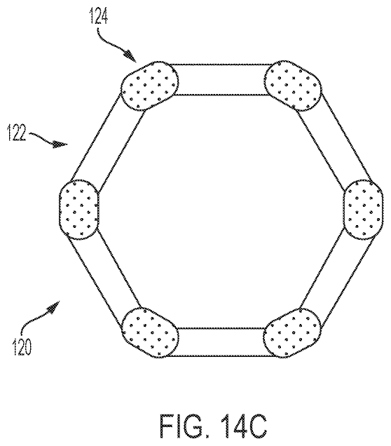

The films are typically applied to segments of the gastric residence systems. The films can also be applied to multi-segment arms prior to attachment of the multi-segment arms to a central elastomer. The films can also be applied to non-segmented arms (that is, arms which comprise only one segment) prior to attachment of the non-segmented arms to a central elastomer. The non-segmented arm can be attached to the central elastomer either directly or via a linker, such as a disintegrating matrix or coupling polymer. An example of segments of a gastric residence system is shown in FIG. 14A, where segment 102 and segment 103 are linked by linker 104, and attached to a central elastomer 106. The segments 102 and 104 comprise carrier polymer and agent (such as a drug). Using a release rate-modulating polymer film on the segments of the gastric residence system provides the advantageous characteristics described herein.

Several parameters of the films can be adjusted in order to generate desired agent release characteristics, and are discussed below

### Chemical Composition of Release Rate-Modulating Polymer Films

Various polymers can be used to form the release-rate modulating polymer films, including PCL, PDL, PDLG, PDL-PCL copolymer, and PVAc. Mixtures of these polymers can also be used. Additional polymers or other compounds can be blended with the base polymer, such as one or more of copovidone, povidone, polyethylene glycol, Pluronic L-31 (PEG-PPG-PEG block co-polymer), polypropylene glycol, polycaprolactone triol, Pluronic F-108 (PEG-PPG-PEG block co-polymer), poly-D-lactide-polycaprolactone co-polymer (25:75), poly-D-lactide-polycaprolactone co-polymer (80:20), propylene glycol, crospovidone, and polyvinylacetate. Ratios of polymers below are expressed in terms of weight (that is, weight/weight; w/w).

Polymers can be characterized by their number-average molecular weight, Mₙ. For example, where a high molecular weight polycaprolactone is desired, polycaprolactone having a number-average molecular weight of about 150,000 to about 250,000, about 175,000 to about 225,000, or about 200,000 can be used. Where a low molecular weight polycaprolactone is desired, polycaprolactone having a number-average molecular weight of about 10,000 to about 30,000, about 15,000 to about 30,000, about 10,000 to about 25,000, about 10,000 to about 20,000, about 12,000 to about 18,000, or about 15,000 can be used.

Polymers can also be characterized by their intrinsic viscosity, which is correlated to molecular weight by the Mark-Houwink equation. For example, polycaprolactone having an intrinsic viscosity of about 1.0 dL/g to about 2.5 dL/g or about 1.5 dL/g to about 2.1 dL/g can be used. The intrinsic viscosity can be measured in CHCl₃ at 25° C. For applications where a high molecular weight PCL is desired, the intrinsic viscosity can be about 1.5 dL/g to about 1.9 dL/g, or the intrinsic viscosity can have a midpoint of about 1.7 dL/g. For applications where a low molecular weight PCL is desired, the intrinsic viscosity can be about 0.2 dL/g to about 0.4 dL/g, or the intrinsic viscosity can have a midpoint of about 0.2 dL/g or 0.4 dL/g.

Poly-D,L-lactide (PDL) is a useful polymer, either alone or in combination with one or more other polymers. In one embodiment, PDL having an intrinsic viscosity of about 1 dl/g to about 5 dl/g can be used. In one embodiment, PDL having an intrinsic viscosity of about 1 dl/g to about 4 dl/g can be used. In one embodiment, PDL having an intrinsic viscosity of about 1 dl/g to about 3 dl/g can be used. In one embodiment, PDL having an intrinsic viscosity of about 1.6 dl/g to about 2.4 dl/g can be used. In another embodiment, PDL having an intrinsic viscosity midpoint of about 2.0 dl/g can be used. In one embodiment, PDL having an intrinsic viscosity of about 1.3 dl/g to about 1.7 dl/g can be used. In another embodiment, PDL having an intrinsic viscosity midpoint of about 1.5 dl/g can be used.

Polymers that can be combined with PDL include poly-D,L-lactide/glycolide (PDLG). In one embodiment, PDLG having an intrinsic viscosity of about 0.1 dl/g to about 3 dl/g, of about 0.1 dl/g to about 1.5 dl/g, or of about 0.1 dl/g to about 0.5 dl/g is used in combination with PDL. A PDL:PDLG ratio of about 9:1 to about 1:3 can be used, such as about 2:1 to about 1:2, about 1.25:1 to about 1:1.25; or about 1:1.

Another polymer that can be combined with PDL includes polycaprolactone (PCL), for example, PCL of molecular weight about Mₙ 75,000 to about Mₙ 250,000.

Another polymer that can be combined with PDL is polyethylene glycol (PEG), such as PEG of molecular weight about Mₙ 800 to about Mₙ 20,000.

Yet another polymer that can be combined with PDL is polypropylene glycol (PPG), such as PPG having Mₙ of at least about 2,500, such as PPG having Mₙ from about 2,500 to about 6,000.

Both PCL and PEG can be combined with PDL, to form a PDL:PCL:PEG film. In one embodiment, the PDL can comprise between about 15% to about 80% of the release rate-modulating film, the PCL can comprise between about 15% to about 75% of the release rate-modulating film, and the PEG can comprise between about 5% to about 15% of the release rate-modulating film, by weight. Exemplary ratios include a PDL:PCL:PEG ratio of about 9:27:4 (w/w/w) and a PDL:PCL:PEG ratio of about 36:9:5 (w/w/w).

PDL:PEG:PPG combinations can also be used. In one embodiment, the PDL can comprise between about 75% to about 95% of the release rate-modulating film, the PEG can comprise between about 3% to about 10% of the release rate-modulating film, and the PPG can comprise between about 1% to about 7% of the release rate-modulating film, by weight.

PDL can also be combined with a polyethylene glycol-polypropylene glycol-polyethylene glycol (PEG-PPG-PEG) block copolymer, for example, a PEG-PPG-PEG block copolymer which comprises about 75% to about 90% ethylene glycol. In one embodiment, the PEG-PPG-PEG block copolymer can have a molecular weight Mₙ of about 14,000 to about 15,000.

Exemplary ratios of this combination include a (PDL):(PEG-PPG-PEG block copolymer) ratio of between about 85:15 to about 95:5 (w/w), and a (PDL):(PEG-PPG-PEG block copolymer) ratio of about 9:1 (w/w).

A PDL-PCL copolymer, that is, poly-D-lactide-polycaprolactone co-polymer, can also be used as a release rate-modulating polymer film. The relative composition of the copolymer can range widely, from about 15% PDL monomer and 85% PCL monomer to about 95% PDL monomer and 5% PCL monomer in the copolymer. Other ranges, such as PDL monomer:PCL monomer of about 15:85 to about 35:65, or about 25:75 and PDL monomer:PCL monomer of about 70:30 to about 90:10, or about 80:20, can be used. The PDL-PCL copolymer can have an intrinsic viscosity of about 0.4 dl/g to about 1.2 dl/g, such as about 0.6 dl/g to about 1 dl/g.

PEG can also be combined with the PDL-PCL copolymer, to form a release rate-modulating polymer film comprising (PDL-PCL copolymer):PEG. The PDL-PCL copolymer can comprise about 75% to about 95% of the release rate modulating film by weight and the PEG can comprise about 5% to about 25% of the release rate modulating film by weight, such as PDL-PCL copolymer comprising about 90% of the release rate modulating film by weight and the PEG comprising about 10% of the release rate modulating film by weight.

Release rate-modulating films comprising high molecular weight poly-D,L-lactide (PDL-HMW) and low molecular weight poly-D,L-lactide (PDL-LMW) can also be used. The PDL-HMW can comprises PDL of inherent viscosity of about 1.6 dl/g to about 5 dl/g, about 1.6 dl/g to about 4 dl/g, or about 1.6 dl/g to about 2.4 dl/g. The PDL-LMW can comprise PDL of inherent viscosity of about 0.5 dl/g to about 1.5 dl/g. PCL and/or PEG can be added to the PDL-HMW/PDL-LMW films.

In alternative embodiments, poly-L-lactide can be used in place of the poly-D,L-lactide in any or all of the embodiments disclosed herein which recite poly-D,L-lactide as a component.

In alternative embodiments, poly-D-lactide can be used in place of the poly-D,L-lactide in any or all of the embodiments disclosed herein which recite poly-D,L-lactide as a component.

Polycaprolactone can be used as a release-rate modulating film. One such formulation comprises both high molecular weight polycaprolactone (PCL-HMW) and low molecular weight polycaprolactone (PCL-LMW). The PCL-HMW can comprise PCL of about Mₙ 75,000 to about Mₙ 250,000; or PCL having an intrinsic viscosity of about 1.0 dl/g to about 2.4 dl/g; or PCL having an intrinsic viscosity of about 1.2 dl/g to about 2.4 dl/g; or PCL having an intrinsic viscosity of about 1.6 dl/g to about 2.4 dl/g. The PCL-LMW can comprise PCL of about Mₙ 10,000 to about Mₙ 20,000; or PCL having an intrinsic viscosity of about 0.1 dl/g to about 0.8 dl/g. Ratios of (PCL-HMW):(PCL-LMW) ratio can range from about 1:4 to about 95:5, about 2:3 to about 95:5, about 3:1 to about 95:5, or about 9:1.

### Advantage of Uniform Release-Rate Modulating Polymer Films During Thermal Processing

Gastric residence systems are often assembled by heating individual components, such as arms and linkers, and pressing the heated components together. Techniques such as infrared welding or contact with a heated platen can be used to heat individual components, which can then be pressed together to join the components.

In some embodiments, release-rate modulating polymer films are applied to gastric residence systems after all heat-assisted assembly steps have been completed. Applying the film after all heat-assisted assembly steps prevents disruption of the film during the heating process. In other embodiments, however, release-rate modulating polymer films are applied to components of gastric residence systems before the all heat-assisted assembly steps have been completed. In these embodiments, it is important that the use of heat during the heat-assisted assembly steps do not change the release-rate properties of the release-rate modulating polymer films.

One aspect of the current disclosure is the use of uniform release-rate modulating polymer films. Uniform films may comprise a single polymer or may comprise multiple polymers, along with other additives such as plasticizers, permeable components, or anti-tack agents. However, all of the ingredients in the film are blended together into a uniform mixture, so that the film, after coating onto any component of the gastric residence system, is essentially uniform. Use of such uniform films is advantageous, as it significantly reduces or prevents alteration of the release rate properties of the release-rate modulating polymer film by any heat-assisted assembly steps.

In some embodiments, the release rate of agent from a coated segment or arm as disclosed herein changes by less than about 20% after heat-assisted assembly, as compared to the release rate of agent from the coated segment or arm before heat-assisted assembly. In some embodiments, the release rate of agent from a coated segment or arm as disclosed herein changes by less than about 15% after heat-assisted assembly, as compared to the release rate of agent from the coated segment or arm before heat-assisted assembly. In some embodiments, the release rate of agent from a coated segment or arm as disclosed herein changes by less than about 10% after heat-assisted assembly, as compared to the release rate of agent from the coated segment or arm before heat-assisted assembly. In some embodiments, the release rate of agent from a coated segment or arm as disclosed herein changes by less than about 5% after heat-assisted assembly, as compared to the release rate of agent from the coated segment or arm before heat-assisted assembly. Comparative release rates can be measured by incubating the coated segment or coated arm in FaSSGF at 37° C., and measuring cumulative release of agent at about day 1, at about day 4, or at about day 7; or at any two of about day 1, about day 4, and about day 7; or at all three of about day 1, about day 4, and about day 7.

Thermal cycling is exposure of an arm, such as an arm coated with a release rate-modulating polymer film, to heat, such as heat-assisted assembly, heat welding, IR welding, or using conditions similar to heat-assisted assembly, heat welding, or IR welding, followed by cooling of the arm. Comparative release rates can be measured as indicated above and in the examples before and after thermal cycling.

Some release-rate modulating polymer films disclosed in WO 2018/227147 contain porogens, which are additives in particle form that dissolve out of the release rate-modulating polymer films, creating pores in the films. Examples of porogens include sodium chloride, sucrose, or water-soluble polymeric materials in particulate form. Use of porogens results in non-uniform (non-homogeneous) release-rate modulating films, where porogen particles or domains are embedded in the release-rate modulating polymer film. Such porogen-containing films may be disrupted during heat-assisted assembly steps. Accordingly, in one embodiment, the release-rate modulating polymer films of the current disclosure do not comprise porogens.

### Plasticizers and Other Additives to Release Rate-Modulating Polymer Films

Plasticizers can also be added to further tune the properties of the release rate-modulating polymer films. Plasticizers that can be used include the classes of phthalates, phosphates, citrates, tartrates, adipates, sebacates, sulfonamides, succinates, glycolates, glycerolates, benzoates, myristates, and halogenated phenyls. Specific plasticizers that can be used include triacetin, triethyl citrate, PEG, poloxamer, tributyl citrate, and dibutyl sebacate. Triacetin and triethyl citrate (TEC) are particularly useful.

Plasticizers can be added to make up about 1% to about 35%, about 1% to about 30%, about 1% to about 25%, about 1% to about 20%, about 1% to about 15%, about 1% to about 10%, about 1% to about 8%, about 1% to about 5%, about 1% to about 3%, about 5% to about 40%, about 10% to about 40%, about 15% to about 40%, about 20% to about 40%, about 25% to about 40%, about 30% to about 40%, about 10% to about 30%, about 15% to about 30%, about 20% to about 30%, about 25% to about 30%, or about 10%, about 15%, about 20%, about 25%, about 30%, about 35%, or about 40% by weight of the release rate-modulating polymer film. A preferred range of plasticizer is about 5% to about 20%, more preferably about 10% to about 20%, by weight of the release rate-modulating polymer film.

Processing aids can also be added to release rate-modulating polymer films. Anti-tack agents, such as magnesium stearate, talc, or glycerol monostearate can be added to aid in processing of the films. Such anti-tack agents can be added in amounts of about 0.5% to about 5%, about 1% to about 3%, or about 2%.

### Film Thickness

The release-rate modulating polymer films should be very thin in comparison to the carrier polymer-agent segment of the gastric residence system that they cover. This allows for diffusion of water into the carrier polymer-agent segment, and diffusion of agent out of the segment.

The thickness of the release-rate modulating polymer films can be between about 1 micrometer to about 40 micrometers, between about 1 micrometer to about 30 micrometers, or between about 1 micrometer to about 25 micrometers. The films are typically between about 1 micrometer to about 20 micrometers, such as between about 1 micrometer to about 20 micrometers, about 1 micrometer to about 15 micrometers, about 1 micrometer to about 10 micrometers, about 1 micrometer to about 5 micrometers, about 1 micrometer to about 4 micrometers, about 1 micrometer to about 3 micrometers, about 1 micrometer to about 2 micrometers, about 2 micrometers to about 10 micrometers, about 5 micrometers to about 20 micrometers, about 5 micrometer to about 10 micrometers, about 10 micrometer to about 15 micrometers, or about 15 micrometers to about 20 micrometers.

In further embodiments, the release-rate modulating polymer film does not add substantially to the strength of the carrier polymer-agent segment that it covers. In further embodiments, the release-rate modulating polymer film adds less than about 20%, less than about 10%, less than about 5%, or less than about 1% to the strength of the segment. The strength of the segment can be measured by the four-point bending flexural test (ASTM D790) described in Example 18 of WO 2017/070612 and Example 13 of WO 2017/100367.

### Film Weight

The release-rate modulating polymer films can be coated onto the carrier polymer-agent arm or arm segment of the gastric residence system in amounts from about 0.1% to 20% of the weight of the carrier polymer-agent arm or arm segment prior to coating; or in amounts from about 0.1% to 15%, of about 0.1% to 10%, about 0.1% to about 8%, about 0.1% to about 5%, about 0.1% to about 4%, about 0.1% to about 3%, about 0.1% to about 2%, about 0.1% to about 1%, about 0.5% to about 10%, about 0.5% to about 8%, about 0.5% to about 5%, about 0.5% to about 4%, about 0.5% to about 3%, about 0.5% to about 2%, about 0.5% to about 1%, about 1% to about 10%, about 1% to about 8%, about 1% to about 5%, about 1% to about 4%, about 1% to about 3%, or about 1% to about 2% of the weight of the carrier polymer-agent arm or arm segment prior to coating. The films can be applied in amounts of about 1% to about 20% of the weight of the carrier polymer-agent arm or arm segment of the gastric residence system prior to coating, such as in amounts of about 1% to about 10%, about 1% to about 7%, about 1% to about 5%, or about 2% to about 5%, for example, in amounts of 1%, 1.5%, 2%, 2.5%, 3%, 3.5%, 4%, 4.5%, 5%, 6%, 7%, 8%, 9%, or 10% of the weight of the carrier polymer-agent arm or arm segment prior to coating.

Application of Release Rate-Modulating Polymer Films onto Segments for Use in Gastric Residence Systems

The release rate-modulating polymer films can be applied to segments for use in gastric residence systems using various techniques. Several of the techniques involve coating a segment, comprising a carrier polymer and agent, with a solution of a formulation of a release rate-modulating polymer film, producing a film-coated segment. The film-coated segment is then dried.

Various methods of coating films onto objects are known in the art, and include dip coating, pan coating, spray coating, and fluidized bed coating. Fluidized bed coating is also known as Wurster coating or air suspension coating. For these coating methods, a formulation of a release-rate modulating polymer film, including the polymer, and any plasticizers if present, is prepared as a solution. The solvent used for the solution of the polymer film formulation is typically an organic solvent, such as ethyl acetate, dichloromethane, acetone, methanol, ethanol, isopropanol, or any combination thereof. Preferably, Class 3 solvents as listed in the guidance from the United States Food and Drug Administration at URL www.fda.gov/downloads/drugs/guidances/ucm073395.pdf (which include ethanol, acetone, and ethyl acetate) are used; however, Class 2 solvents (which include dichloromethane and methanol) can be used if necessary for the formulation. Class 1 and Class 4 solvents should be used only when the formulation cannot be prepared with a suitable Class 3 or Class 2 solvent.

Release rate-modulating polymer films can also be integrated onto segments by co-extrusion, where the segment formulation is co-extruded with a surrounding thin layer of the release rate-modulating polymer film.

The Examples below illustrate the use of some of these coating techniques for preparation of segments with a release rate-modulating polymer film.

### Overall System Configuration

The current disclosure provides, inter alia, gastric residence systems, arms of gastric residence systems, and segments for use in gastric residence systems and arms of gastric residence systems, which are coated with a release rate-modulating film. As discussed, the release rate-modulating film provides a number of advantages.

Gastric residence systems can be prepared in different configurations. The “stellate” configuration of a gastric residence system is also known as a “star” (or “asterisk”) configuration. An example of a stellate system 100 is shown schematically in FIG. 14A. Multiple arms (also called “elongate members”; only one such arm, 108, is labeled for clarity), are affixed to disk-shaped central elastomer 106. The arms depicted in FIG. 14A are comprised of segments 102 and 103, joined by a coupling polymer or linker region 104 (again, the components are only labeled in one arm for clarity) which serves as a linker region. This configuration permits the system to be folded or compacted at the central elastomer. FIG. 14B shows a folded configuration 190 of the gastric residence system of FIG. 14A (for clarity, only two arms are illustrated in FIG. 14B). Segments 192 and 193, linker region 194, elastomer 196, and arm 198 of FIG. 14B correspond to segments 102 and 103, linker region 104, elastomer 106, and arm 108 of FIG. 14A, respectively. When folded, the overall length of the system is reduced by approximately a factor of two, and the system can be conveniently placed in a container such as a capsule or other container suitable for oral administration. When the capsule reaches the stomach, the capsule dissolves, releasing the gastric residence system. The gastric residence system then unfolds into its uncompacted state, which is retained in the stomach for the desired residence period.

While the linker regions 104 are shown as slightly larger in diameter than the segments 102 and 103 in FIG. 14A, they can be the same diameter as the segments, so that the entire arm 102-104-103 has a smooth outer surface.

In some embodiments, the stellate system may have an arm composed of only one segment, which is attached to the central elastomer by a linker region. This corresponds to FIG. 14A with the segments 103 omitted. The single-segment arms comprising segments 102 are then directly attached to central elastomer 106 via the linkers 104. The linkers can comprise a coupling polymer or a disintegrating matrix.

The stellate system thus constitutes at least three arms, where one or more arms is coated with a release rate-modulating polymer film as described herein; and a central elastic polymeric component. The one or more arms are each connected to the central elastic polymeric component via a separate linker component. The gastric residence system is configured to be folded and physically constrained during administration and is configured to assume an open retention shape upon removal of a constraint. Change between the folded shape and the open retention shape is mediated by the elastic polymeric component that undergoes elastic deformation when the residence system is in the folded shape and recoils when the gastric residence system assumes the open retention shape. The linker degrades, dissolves, disassociates, or mechanically weakens in a gastric environment, which results in loss of retention shape integrity and passage out of a gastric cavity.

FIG. 14C shows another possible overall configuration 120 for a gastric residence system, which is a ring configuration. Segments 122 are joined by coupling polymer or linker region 124 (only one segment and one coupling linkage are labeled for clarity). The coupling polymer/linker region in this design must also function as an elastomer, to enable the ring to be twisted into a compacted state for placement in a container, such as a capsule. The segments depicted constitute the arms in this ring configuration of the gastric residence system.

In one embodiment of the stellate configuration, the segments 102 and 103 comprise a carrier polymer blended with an agent or drug. In one embodiment of the ring configuration, the segments 122 comprise a carrier polymer blended with an agent or drug.

### Evaluation of Release Characteristics

The release characteristics of agent from segments, arms, and gastric residence systems can be evaluated by various assays. Assays for agent release are described in detail in the examples. Release of agent in vitro from segments, arms, and gastric residence systems can be measured by immersing a segment, arm, or gastric residence system in a liquid, such as distilled water, 0.1 N HCl, buffered solutions, fasted state simulated gastric fluid (FaSSGF), or fed state simulated gastric fluid (FeSSGF). Fasted state simulated gastric fluid (FaSSGF) is a preferred aqueous medium for release assays. Simulated gastric fluid indicates either fasted state simulated gastric fluid (FaSSGF) or fed state simulated gastric fluid (FeSSGF); when a limitation is specified as being measured in simulated gastric fluid (SGF), the limitation is met if the limitation holds in either fasted state simulated gastric fluid (FaSSGF) or fed state simulated gastric fluid (FeSSGF). For example, if a segment is indicated as releasing at least 10% of an agent over the first 24 hours in simulated gastric fluid, the limitation is met if the segment releases at least 10% of the agent over the first 24 hours in fasted state simulated gastric fluid, or if the segment releases at least 10% of the agent over the first 24 hours in fed state simulated gastric fluid.). Release rates can be measured at any desired temperature, which will typically be in a range from about 35° C. to about 40° C., such as normal body temperature of 37° C. Release rates can be measured for any desired period of time, for example, about 30 minutes, about 1, 2, 3, 4, 5, 6, 10, 12, 15, 18, 20, or 24 hours; about 1, 2, 3, 4, 5, 6, or 7 days; about 1, 2, 3, or 4 weeks; or about 1 month. When in vitro tests are done to compare release rates, the comparison solutions are kept at the same temperature, such as room temperature, 25° C., or 37° C. Room temperature (ambient temperature) is a preferred temperature for measurements or comparisons; in one embodiment, the ambient temperature does not drop below 20° C. or exceed 25° C. (although it may fluctuate between 20° C. and 25° C.). Normal human body temperature (37° C.) is another preferred temperature for measurements or comparisons.

Release rates can also be measured in environments designed to test specific conditions, such as an environment designed to simulate consumption of alcoholic beverages. Such environments can comprise a mixture of any one of the aqueous solutions described herein and ethanol, for example, a mixture of about 60% of any one of the aqueous solutions described herein and about 40% ethanol. Sequential exposure to different aqueous media (that is, different environments) can also be used to measure release rates.

Fasted state simulated gastric fluid (FaSSGF) is typically prepared using Biorelevant powders (biorelevant.com; Biorelevant.com Ltd., London, United Kingdom). When FaSSGF is prepared according to the Biorelevant “recipe,” it is an aqueous solution at pH 1.6 with taurocholate (0.08 mM), phospholipids (0.02 mM), sodium (34 mM), and chloride (59 mM).

In vivo tests can be performed in animals such as dogs (for example, beagle dogs or hound dogs) and swine. For in vivo tests, a gastric residence system is used, since an individual segment or arm would not be retained in the stomach of the animal. Blood samples can be obtained at appropriate time points, and, if desired, gastric contents can be sampled by cannula or other technique.

Clinical trials in humans, conducted in accordance with appropriate laws, regulations, and institutional guidelines, also provide in vivo data.

### Release Profiles

The increased linearity profiles of the segments with release rate-modulating polymer films provides advantageous release characteristics over a segment with the same carrier polymer-agent composition, but lacking the release rate-modulating polymer films. For example, a segment of a gastric residence system comprising a carrier polymer, an agent or a salt thereof, and a release-rate modulating polymer film configured to control the release rate of the agent, can have a release profile where the release-rate modulating polymer film is configured such that, over a seven-day incubation in simulated gastric fluid, the amount of the agent or salt thereof released during day 5 is at least about 40% of the amount of agent or salt thereof released during day 2. That is, over the seven day incubation period, the amount of the agent or salt thereof released from hours 96-120 (day 5) is at least about 40% of the amount of agent or salt released during hours 24-48 (day 2) of the incubation. In some embodiments, release over day 5 is at least about 50%, at least about 60%, at least about 70%, at least about 80%, or at least about 90% of the amount of agent or salt released over day 2. In some embodiments, release over day 5 is at least about 40% to about 90%, at least about 50% to about 90%, at least about 60% to about 90%, at least about 70% to about 90%, at least about 80% to about 90%, or at least about 40% to about 100%, of the amount of agent or salt released over day 2. In any of these embodiments, at least about 5% of the total amount of agent is released on day 2 and at least about 5% of the total amount of agent is released on day 5, at least about 5% of the total amount of agent is released on day 2 and at least about 7% of the total amount of agent is released on day 5, or at least about 7% of the total amount of agent is released on day 2 and at least about 7% of the total amount of agent is released on day 5. “Total amount of agent” refers to the amount of agent originally present in the segment.

In another embodiment, a segment of a gastric residence system comprising a carrier polymer, an agent or a salt thereof, and a release-rate modulating polymer film configured to control the release rate of the agent, can have a release profile where the release-rate modulating polymer film is configured such that, over a seven-day incubation in simulated gastric fluid, the amount of the agent or salt thereof released during day 7 is at least about 20% of the amount of agent or salt thereof released during day 1. That is, over the seven day incubation period, the amount of the agent or salt thereof released from hours 144-168 (day 7) is at least about 20% of the amount of agent or salt released during hours 0-24 (day 1) of the incubation. In some embodiments, release over day 7 is at least about 30%, at least about 40%, at least about 50%, at least about 60%, or at least about 70% of the amount of agent or salt released over day 1. In some embodiments, release over day 7 is at least about 20% to about 70%, at least about 30% to about 70%, at least about 40% to about 70%, at least about 50% to about 70%, at least about 60% to about 70%, or at least about 20% to about 100%, of the amount of agent or salt released over day 1. In any of these embodiments, at least about 7% of the total amount of agent is released on day 1 and at least about 4% of the total amount of agent is released on day 7, at least about 4% of the total amount of agent is released on day 1 and at least about 4% of the total amount of agent is released on day 7, or at least about 7% of the total amount of agent is released on day 1 and at least about 7% of the total amount of agent is released on day 7. “Total amount of agent” refers to the amount of agent originally present in the segment.

Segments with release rate-modulating polymer films as disclosed herein also have lower burst release when initially immersed in simulated gastric fluid. In one embodiment, a segment of a gastric residence system comprising a carrier polymer and an agent or a salt thereof, where the segment has a release-rate modulating polymer film configured to control the release rate of the agent, can have a release profile where the release-rate modulating polymer film is configured such that the release of agent from the segment in simulated gastric fluid over an initial 24 hour period is at least about 40% lower than the release of agent from a second segment in simulated gastric fluid over an initial 6 hour period, where the second segment comprises the same combination of carrier polymer and agent or salt thereof, but lacks the release-rate modulating polymer film; and wherein the release of agent from the segment with the polymer film in simulated gastric fluid over a seven-day period is either i) at least about 60% of the release of agent from the second segment lacking the polymer film over a seven-day period, or ii) at least 60% of the total amount of agent originally present in the segment. In further embodiments, the release of agent from the segment with the film in simulated gastric fluid over an initial 24 hour period is at least about 40% lower, about 40% to about 50% lower, about 40% to about 60% lower, or about 40% to about 70% lower than the release of agent from a second segment without the film in simulated gastric fluid over an initial 6 hour period, while the release of agent from the segment with the film in simulated gastric fluid over a seven day period is either i) at least about 60%, at least about 70%, at least about 80%, or about 60% to about 80% of the release of agent from the second segment in simulated gastric fluid lacking the polymer film over a seven-day period, or ii) at least about 60%, at least about 70%, at least about 80%, or about 60% to about 80% of the total amount of agent originally present in the segment. In further embodiments, the release of agent from the segment with the film in simulated gastric fluid over a seven-day period is either i) at least about 60%, at least about 70%, at least about 75%, or at least about 80% (such as about 60% to about 70%, about 60% to about 80%, about 60% to about 90%, or about 60% to about 99%) of the release of agent from the second segment without the film in simulated gastric fluid over a seven-day period, or ii) at least about 60%, at least about 70%, at least about 75%, or at least about 80% (such as about 60% to about 70%, about 60% to about 80%, about 60% to about 90%, or about 60% to about 99%) of the total amount of agent originally present in the segment.

Linearity of release of agent from segments having a release rate-modulating polymer film coating is also improved. In one embodiment, a segment of a gastric residence system comprising a carrier polymer and an agent or a salt thereof, where the segment has a release-rate modulating polymer film configured to control the release rate of the agent, can have a release profile where the release-rate modulating polymer film is configured such that a best-fit linear regression model of the release rate of agent has a coefficient of determination R² of at least about 0.8, at least about 0.85, or at least about 0.9 over an initial period of seven days in simulated gastric fluid (where the initial period of seven days is measured from the start time when the segment is initially immersed in simulated gastric fluid; that is, the period of seven days includes the time at t=0 or origin point of the release profile); and wherein the segment releases about 30% to about 70% of the agent or salt thereof within a time of about 40% to about 60% of the seven-day period.

In one embodiment, a segment of a gastric residence system comprising a carrier polymer and an agent or a salt thereof, where the segment has a release-rate modulating polymer film configured to control the release rate of the agent, can have a release profile where the release-rate modulating polymer film is configured such that the release rate over any one of the seven days varies by no more than about 50%, no more than about 40%, no more than about 30%, no more than about 25%, no more than about 20%, or no more than about 10% from the average daily total release over the seven days.

### Carrier Polymers for Segments and Arms (Carrier Polymer-Agent Component)

Exemplary carrier polymers suitable for use in the release-rate modulating polymer films disclosed herein include, but are not limited to, hydrophilic cellulose derivatives (such as hydroxypropylmethyl cellulose, hydroxypropyl cellulose, hydroxymethyl cellulose, hydroxyethyl cellulose, carboxymethylcellulose, sodium-carboxymethylcellulose), cellulose acetate phthalate, poly(vinyl pyrrolidone), ethylene/vinyl alcohol copolymer, poly(vinyl alcohol), carboxyvinyl polymer (Carbomer), Carbopol® acidic carboxy polymer, polycarbophil, poly(ethyleneoxide) (Polyox WSR), polysaccharides and their derivatives, polyalkylene oxides, polyethylene glycols, chitosan, alginates, pectins, acacia, tragacanth, guar gum, locust bean gum, vinylpyrrolidonevinyl acetate copolymer, dextrans, natural gum, agar, agarose, sodium alginate, carrageenan, fucoidan, furcellaran, laminaran, hypnea, eucheuma, gum arabic, gum ghatti, gum karaya, arbinoglactan, amylopectin, gelatin, gellan, hyaluronic acid, pullulan, scleroglucan, xanthan, xyloglucan, maleic anhydride copolymers, ethylenemaleic anhydride copolymer, poly(hydroxyethyl methacrylate), ammoniomethacrylate copolymers (such as Eudragit RL or Eudragit RS), poly(ethylacrylate-methylmethacrylate) (Eudragit NE), Eudragit E (cationic copolymer based on dimethylamino ethyl methylacrylate and neutral methylacrylic acid esters), poly(acrylic acid), polymethacrylates/polyethacrylates such as poly(methacrylic acid), methylmethacrylates, and ethyl acrylates, polylactones such as poly(caprolactone), polyanhydrides such as poly[bis-(p-carboxyphenoxy)-propane anhydride], poly(terephthalic acid anhydride), polypeptides such as polylysine, polyglutamic acid, poly(ortho esters) such as copolymers of DETOSU with diols such as hexane diol, decane diol, cyclohexanedimethanol, ethylene glycol, polyethylene glycol and incorporated herein by reference those poly(ortho) esters described and disclosed in U.S. Pat. No. 4,304,767, starch, in particular pregelatinized starch, and starch-based polymers, carbomer, maltodextrins, amylomaltodextrins, dextrans, poly(2-ethyl-2-oxazoline), poly(ethyleneimine), polyurethane, poly(lactic acid), poly(glycolic acid), poly(lactic-co-glycolic acid) (PLGA), polyhydroxyalkanoates, polyhydroxybutyrate, and copolymers, mixtures, blends and combinations thereof. Polycaprolactone (PCL) is a useful carrier polymer. In another embodiment, polydioxanone is used as the carrier polymer. In any of the embodiments of the gastric residence system, the carrier polymer used in the gastric residence system can comprise polycaprolactone, such as linear polycaprolactone with a number-average molecular weight (Mn) range between about 60 kiloDalton (kDa) to about 100 kDa; 75 kDa to 85 kDa; or about 80 kDa; or between about 45 kDa to about 55 kDa; or between about 50 kDa to about 110,000 kDa, or between about 80 kDa to about 110,000 kDa.

Other excipients can be added to the carrier polymers to modulate the release of agent. Such excipients can be added in amounts from about 1% to 15%, preferably from about 5% to 10%, more preferably about 5% or about 10%. Examples of such excipients include Poloxamer 407 (available as Kolliphor P407, Sigma Cat #62035), poly(ethylene glycol)-block-poly(propylene glycol)-block-poly(ethylene glycol), CAS No. 9003-11-6; H—(OCH2CH2)x-(O—CH(CH3)CH2)y-(OCH2CH2)z-OH where x and z are about 101 and y is about 56); Pluronic P407; Eudragit E, Eudragit EPO (dimethylaminoethyl methacrylate-butyl methacrylate-methyl methacrylate copolymer; available from Evonik); hypromellose (available from Sigma, Cat #H3785), Kolliphor RH40 (available from Sigma, Cat #07076), polyvinyl caprolactam, polyvinyl acetate (PVAc), polyvinylpyrrolidone (PVP), polyvinyl alcohol (PVA), polyethylene glycol (PEG), PDO (polydioxanone), Kollidon VA64 (copovidone; vinylpyrrolidone—vinyl acetate copolymer in a ratio of 6:4 by mass), and Soluplus (available from BASF; a copolymer of polyvinyl caprolactam, polyvinyl acetate, and polyethylene glycol). Preferred soluble excipients include Eudragit E, polyethylene glycol (PEG), polyvinylpyrrolidone (PVP), polyvinyl acetate (PVAc), and polyvinyl alcohol (PVA). Preferred insoluble excipients include Eudragit RS and Eudragit RL. Preferred insoluble, swellable excipients include crospovidone, croscarmellose, hypromellose acetate succinate (HPMCAS), and carbopol. EUDRAGIT RS and EUDRAGIT RL are registered trademarks of Evonik (Darmstadt, Germany) for copolymers of ethyl acrylate, methyl methacrylate and methacrylic acid ester with quaternary ammonium groups (trimethylammonioethyl methacrylate chloride), having a molar ratio of ethyl acrylate, methyl methacrylate and trimethylammonioethyl methacrylate of about 1:2:0.2 in Eudragit® RL and about 1:2:0.1 in Eudragit® RS. Preferred insoluble, swellable excipients include crospovidone, croscarmellose, hypromellose acetate succinate (HPMCAS), carbopol, and linear block copolymers of dioxanone and ethylene glycol; linear block copolymers of lactide and ethylene glycol; linear block copolymers of lactide, ethylene glycol, trimethyl carbonate, and caprolactone; linear block copolymers of lactide, glycolide, and ethylene glycol; linear block copolymers of glycolide, polyethylene glycol, and ethylene glycol; such as linear block copolymers of dioxanone (80%) and ethylene glycol (20%); linear block copolymers of lactide (60%) and ethylene glycol (40%); linear block copolymers of lactide (68%), ethylene glycol (20%), trimethyl carbonate (10%), and caprolactone (2%); linear block copolymers of lactide (88%), glycolide (8%), and ethylene glycol (4%); linear block copolymers of glycolide (67%), polyethylene glycol (28%), and ethylene glycol (5%).

Further examples of excipients that can be used in the segments of the gastric residence system are listed in the Excipient Table below.

Excipient Table

| Function | General examples | Specific examples |
|---|---|---|
| Polymeric and non-polymeric | Polyalkylene oxides | Kolliphor RH, Kolliphor P407, |
| solubilizers | Polyethoxylated castor oil | Soluplus, Cremophor, SDS |
| | Detergents | |
| Release-enhancing excipient | Acrylate polymers | Eudragit RL |
| (wicking agent) | Acrylate co-polymers | Eudragit RS |
| | Polyvinylpyrrolidone | Eudragit E |
| | | Linear block copolymer of |
| | | dioxanone and ethylene glycol |
| | | (e.g., 80:20 ratio) |
| Dispersant | porous inorganic material | silica, hydrophilic-fumed silica, |
| | polar inorganic material | hydrophobic colloidal silica, |
| | non-toxic metal oxides | magnesium aluminum silicate, |
| | amphiphilic organic molecules | stearate salts, calcium stearate, |
| | polysaccharides, cellulose, cellulose | magnesium stearate, |
| | derivatives | microcrystalline cellulose, |
| | fatty acids | carboxymethylcellulose, |
| | detergents | hypromellose, phospholipids, |
| | | polyoxyethylene stearates, zinc |
| | | acetate, alginic acid, lecithin, |
| | | sodium lauryl sulfate, aluminum |
| | | oxide |
| Stabilizer/Preservative agent | Anti-oxidants | Tocopherols |
| | Anti-microbial agents | Alpha-tocopherol |
| | Buffering substances/pH stabilizers | Ascorbic acid; ascorbate salts |
| | | Carotenes |
| | | Butylated hydroxytoluene (BHT) |
| | | Butylated hydroxyanisole (BHA) |
| | | Fumaric acid |
| | | calcium carbonate |
| | | calcium lactate |
| | | calcium phosphate |
| | | sodium phosphate |
| | | sodium bicarbonate |

### Agents for Use in Gastric Residence Systems

Agents which can be administered to or via the gastrointestinal tract can be used in the gastric residence systems as disclosed herein. The agent is blended with the carrier polymer, and any other excipients or other additives to the carrier polymer, and formed into a segment for use in a gastric residence system. Agents include, but are not limited to, drugs, pro-drugs, biologics, and any other substance which can be administered to produce a beneficial effect on an illness or injury. Agents that can be used in the gastric residence systems as disclosed herein include statins, such as rosuvastatin; nonsteroidal anti-inflammatory drugs (NSAIDs) such as meloxicam; selective serotonin reuptake inhibitors (SSRIs) such as escitalopram and citalopram; blood thinners, such as clopidogrel; steroids, such as prednisone; antipsychotics, such as aripiprazole and risperidone; analgesics, such as buprenorphine; opioid antagonists, such as naloxone; anti-asthmatics such as montelukast; anti-dementia drugs, such as memantine; cardiac glycosides such as digoxin; alpha blockers such as tamsulosin; cholesterol absorption inhibitors such as ezetimibe; anti-gout treatments, such as colchicine; antihistamines, such as loratadine and cetirizine, opioids, such as loperamide; proton-pump inhibitors, such as omeprazole; antiviral agents, such as entecavir; antibiotics, such as doxycycline, ciprofloxacin, and azithromycin; anti-malarial agents; levothyroxine; substance abuse treatments, such as methadone and varenicline; contraceptives; stimulants, such as caffeine; and nutrients such as folic acid, calcium, iodine, iron, zinc, thiamine, niacin, vitamin C, vitamin D, biotin, plant extracts, phytohormones, and other vitamins or minerals. Biologics that can be used as agents in the gastric residence systems disclosed herein include proteins, polypeptides, polynucleotides, and hormones. Exemplary classes of agents include, but are not limited to, analgesics; anti-analgesics; anti-inflammatory drugs; antipyretics; antidepressants; antiepileptics; antipsychotic agents; neuroprotective agents; anti-proliferatives, such as anti-cancer agents; antihistamines; antimigraine drugs; hormones; prostaglandins; antimicrobials, such as antibiotics, antifungals, antivirals, and antiparasitics; anti-muscarinics; anxiolytics; bacteriostatics; immunosuppressant agents; sedatives; hypnotics; antipsychotics; bronchodilators; anti-asthma drugs; cardiovascular drugs; anesthetics; anti-coagulants; enzyme inhibitors; steroidal agents; steroidal or non-steroidal anti-inflammatory agents; corticosteroids; dopaminergics; electrolytes; gastro-intestinal drugs; muscle relaxants; nutritional agents; vitamins; parasympathomimetics; stimulants; anorectics; anti-narcoleptics; and antimalarial drugs, such as quinine, lumefantrine, chloroquine, amodiaquine, pyrimethamine, proguanil, chlorproguanil-dapsone, sulfonamides (such as sulfadoxine and sulfamethoxypyridazine), mefloquine, atovaquone, primaquine, halofantrine, doxycycline, clindamycin, artemisinin, and artemisinin derivatives (such as artemether, dihydroartemisinin, arteether and artesunate). The term “agent” includes salts, solvates, polymorphs, and co-crystals of the aforementioned substances. In certain embodiments, the agent is selected from the group consisting of cetirizine, rosuvastatin, escitalopram, citalopram, risperidone, olanzapine, donepezil, and ivermectin. In another embodiment, the agent is one that is used to treat a neuropsychiatric disorder, such as an anti-psychotic agent, or an anti-dementia drug such as memantine.

### Agent Loading of Arms and Segments

The arms, or segments of which the arms are comprised, comprise agent or a pharmaceutically acceptable salt thereof. In some embodiments, the agent or salt thereof (for example, a drug) makes up about 10% to about 40% by weight of the arm or segment, and thus the carrier polymer and any other components of the arm or segment blended into the carrier polymer together make up the remainder of the weight of the arm or segment. In some embodiments, the agent or salt thereof makes up about 10% to about 35%, about 10% to about 30%, about 10% to about 25%, about 10% to about 20%, about 10% to about 15%, about 15% to about 40%, about 20% to about 40%, about 25% to about 40%, about 30% to about 40%, about 35% to about 40%, about 15% to about 35%, about 20% to about 35%, or about 25% to about 40% by weight of the arm or segment.

In some embodiments, the amount of agent by weight in the arms, or segments of which the arms are comprised, can comprise about 20% to about 60%, about 25% to about 60%, about 30% to about 60%, about 35% to about 60%, about 20% to about 50%, about 20% to about 40%, or about 25% to about 50%.

In some embodiments, the amount of agent by weight in the arms, or segments of which the arms are comprised, can comprise at least about 40%, at least about 45%, at least about 50%, at least about 55%, or about 60%. In some embodiments, the amount of agent by weight in the arms, or segments of which the arms are comprised, can comprise about 40% to about 60%, about 45% to about 60%, about 50% to about 60%, about 55% to about 60%, about 40% to about 55%, about 40% to about 50%, or about 40% to about 45%. In some embodiments, the amount of agent by weight in the arms, or segments of which the arms are comprised, can comprise about 25% to about 60%, about 30% to about 60%, or about 35% to about 60%. In some embodiments, the amount of agent by weight in the arms, or segments of which the arms are comprised, can comprise about 51% to about 60%, about 52% to about 60%, about 53% to about 60%, about 54% to about 60%, about 55% to about 60%, about 56% to about 60%, or about 57% to about 60%. In some embodiments, the agent or pharmaceutically acceptable salt thereof is present in an amount by weight of between about 67% and about 150% of the weight of the carrier polymer.

### Dispersants for Use in Gastric Residence Systems

Dispersants can be used in the gastric residence systems in order to improve distribution of agent in the carrier polymer-agent arms and provide more consistent release characteristics. Examples of dispersants that can be used include silicon dioxide (silica, SiO₂) (including hydrophilic fumed silica); stearate salts, such as calcium stearate and magnesium stearate; microcrystalline cellulose; carboxymethylcellulose; hydrophobic colloidal silica; hypromellose; magnesium aluminum silicate; phospholipids; polyoxyethylene stearates; zinc acetate; alginic acid; lecithin; fatty acids; sodium lauryl sulfate; and non-toxic metal oxides such as aluminum oxide. Porous inorganic materials and polar inorganic materials can be used. Hydrophilic-fumed silicon dioxide is a preferred dispersant. One particularly useful silicon dioxide is sold by Cabot Corporation (Boston, Mass., USA) under the registered trademark CAB-O-SIL® M-5P (CAS #112945-52-5), which is hydrophilic-fumed silicon dioxide having a BET surface area of about 200 m2/g±15 m2/g The mesh residue for this product on a 45 micron sieve is less than about 0.02%. The typical primary aggregate size is about 150 to about 300 nm, while individual particle sizes may range from about 5 nm to about 50 nm.

The weight/weight ratio of dispersant to agent substance can be about 0.1% to about 5%, about 0.1% to about 4%, about 0.1% to about 3%, about 0.1% to about 2%, about 0.1% to about 1%, about 1% to about 5%, about 1% to about 4%, about 1% to about 3%, about 1% to about 2%, about 2% to about 4%, about 2% to about 3%, about 3% to about 4%, about 4% to about 5%, or about 0.1%, about 0.5%, about 1%, about 2%, about 3%, about 4% or about 5%.

Dispersants can comprise about 0.1% to about 4% of the carrier polymer-agent components, such as about 0.1% to about 3.5%, about 0.1% to about 3%, about 0.1% to about 2.5%, about 0.1% to about 2%, about 0.1% to about 1.5%, about 0.1% to about 1%, about 0.1% to about 0.5%, or about 0.2% to about 0.8%.

### Stabilizers for Use in Gastric Residence Systems

Many agents are prone to oxidative degradation when exposed to reactive oxygen species, which can be present in the stomach. An agent contained in the system may thus oxidize due to the prolonged residence in the stomach of the system, and the extended release period of agent from the system. Accordingly, it is desirable to include stabilizers, such as anti-oxidants including tocopherols, alpha-tocopherol, ascorbic acid, ascorbyl palmitate, butylated hydroxytoluene, butylated hydroxyanisole, and fumaric acid, in the systems, in amounts of about 0.1% to about 4% of the carrier polymer-agent components, such as about 0.1% to about 3.5%, about 0.1% to about 3%, about 0.1% to about 2.5%, about 0.1% to about 2%, about 0.1% to about 1.5%, about 0.1% to about 1%, about 0.1% to about 0.5%, or about 0.2% to about 0.8%. Vitamin E, a tocopherol, a Vitamin E ester, a tocopherol ester, ascorbic acid, or a carotene, such as alpha-tocopherol, Vitamin E succinate, alpha-tocopherol succinate, Vitamin E acetate, alpha-tocopherol acetate, Vitamin E nicotinate, alpha-tocopherol nicotinate, Vitamin E linoleate, or alpha-tocopherol linoleate can be used as anti-oxidant stabilizers.

Buffering or pH-stabilizer compounds that can be included in the systems to reduce or prevent degradation of pH-sensitive agents at low pH include calcium carbonate, calcium lactate, calcium phosphate, sodium phosphate, and sodium bicarbonate. They are typically used in an amount of up to about 2% w/w. The buffering or pH-stabilizer compounds can comprise about 0.1% to about 4% of the carrier polymer-agent components, such as about 0.1% to about 3.5%, about 0.1% to about 3%, about 0.1% to about 2.5%, about 0.1% to about 2%, about 0.1% to about 1.5%, about 0.10% to about 10%, about 0.10% to about 0.5%, or about 0.2% to about 0.8%. The anti-oxidant stabilizers, pH stabilizers, and/or other stabilizer compounds can be blended into the carrier polymer, the agent, or the carrier polymer-agent mixture, resulting in the presence of the anti-oxidant stabilizers, pH stabilizers, and/or other stabilizer compounds in the final segment or arm.

### Residence Time

The residence time of the gastric residence system is defined as the time between administration of the system to the stomach and exit of the system from the stomach. In one embodiment, the gastric residence system has a residence time of about 24 hours, or up to about 24 hours. In one embodiment, the gastric residence system has a residence time of about 48 hours, or up to about 48 hours. In one embodiment, the gastric residence system has a residence time of about 72 hours, or up to about 72 hours. In one embodiment, the gastric residence system has a residence time of about 96 hours, or up to about 96 hours. In one embodiment, the gastric residence system has a residence time of about 5 days, or up to about 5 days. In one embodiment, the gastric residence system has a residence time of about 6 days, or up to about 6 days. In one embodiment, the gastric residence system has a residence time of about 7 days (about one week), or up to about 7 days (about one week). In one embodiment, the gastric residence system has a residence time of about 10 days, or up to about 10 days. In one embodiment, the gastric residence system has a residence time of about 14 days (about two weeks), or up to about 14 days (about two weeks). In one embodiment, the gastric residence system has a residence time of about 21 days (about three weeks), or up to about 21 days (about three weeks). In one embodiment, the gastric residence system has a residence time of about 28 days (about four weeks), or up to about 28 days (about four weeks). In one embodiment, the gastric residence system has a residence time of about a month, or up to about a month.

The gastric residence system releases a therapeutically effective amount of agent (or salt thereof) during at least a portion of the residence time or residence period during which the system resides in the stomach. In one embodiment, the system releases a therapeutically effective amount of agent (or salt thereof) during at least about 25% of the residence time. In one embodiment, the system releases a therapeutically effective amount of agent (or salt thereof) during at least about 50% of the residence time. In one embodiment, the system releases a therapeutically effective amount of agent (or salt thereof) during at least about 60% of the residence time. In one embodiment, the system releases a therapeutically effective amount of agent (or salt thereof) during at least about 70% of the residence time. In one embodiment, the system releases a therapeutically effective amount of agent (or salt thereof) during at least about 75% of the residence time. In one embodiment, the system releases a therapeutically effective amount of agent (or salt thereof) during at least about 80% of the residence time. In one embodiment, the system releases a therapeutically effective amount of agent (or salt thereof) during at least about 85% of the residence time. In one embodiment, the system releases a therapeutically effective amount of agent (or salt thereof) during at least about 90% of the residence time. In one embodiment, the system releases a therapeutically effective amount of agent (or salt thereof) during at least about 95% of the residence time. In one embodiment, the system releases a therapeutically effective amount of agent (or salt thereof) during at least about 98% of the residence time. In one embodiment, the system releases a therapeutically effective amount of agent (or salt thereof) during at least about 99% of the residence time.

### Radiopacity

The systems are optionally radiopaque, so that they can be located via abdominal X-ray if necessary. In some embodiments, one or more of the materials used for construction of the system is sufficiently radiopaque for X-ray visualization. In other embodiments, a radiopaque substance is added to one or more materials of the system, or coated onto one or more materials of the system, or are added to a small portion of the system. Examples of suitable radiopaque substances are barium sulfate, bismuth subcarbonate, bismuth oxychloride, and bismuth trioxide. It is preferable that these materials should not be blended into the polymers used to construct the gastric residence system, so as not to alter drug release from the carrier polymer, or desired properties of other system polymers. Metal striping or tips on a small portion of the system components can also be used, such as tungsten.

### Manufacture/Assembly of System Using Heat-Assisted Assembly and Infrared Welding

Components of the gastric residence systems can be manufactured by various methods, such as co-extrusion or three-dimensional printing, as disclosed in U.S. Pat. No. 10,182,985, and published patent applications US 2018/0311154 A1, US 2019/0262265 A1, US 2019/0231697 A1, US 2019/0254966 A1, and WO 2018/227147.

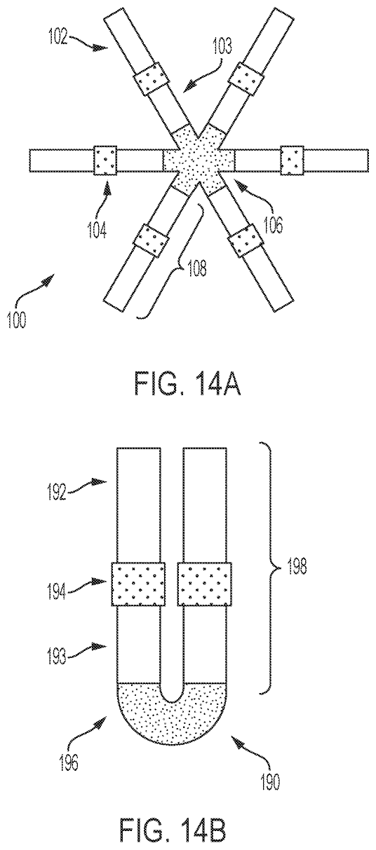

FIG. 13 shows an exemplary method of bonding components together to form a gastric residence system. A pre-cut polymeric linker (such as an enteric linker or a time-dependent linker) is laser or IR welded to an elastomeric central member. The polymeric linker may be formed, for example, by hot melt extruding a material and cutting it to the desired length. Hot melt extruded arms (elongate members) containing a carrier polymer mixed with an agent are then laser or IR welded to the polymeric linkers, thereby forming the stellate structure of the gastric residence system.

Heat-assisted assembly can be accomplished by contacting the surfaces to be joined with a heated platen, by using an infrared radiation source such as an infrared lamp, by using an infrared laser, or by using other heat-producing, heat-emitting, or heat-transferring devices. Examples 12-14 of US 2019/0262265 A1 describe modalities for heating components of gastric residence system, such as by using a hot plate or an infrared lamp. The heated surfaces are then pressed together, followed by cooling.

Infrared welding can be performed by contacting a first surface on a first component with a second surface on a second component, and irradiating the region of the contacting surfaces with infrared radiation, while applying force or pressure to maintain the contact between the two surfaces, followed by cooling of the attached components (the applied force or pressure is optionally maintained during the cooling process).

### Methods of Treatment Using the Gastric Residence Systems

The gastric residence systems can be used to treat conditions requiring administration of a drug or agent over an extended period of time. In a preferred embodiment, a gastric residence system is administered to a human. For long-term administration of agents or drugs which are taken for months, years, or indefinitely, administration of a gastric residence system periodically, such as once weekly or once every two weeks can provide substantial advantages in patient compliance and convenience. Accordingly, the gastric residence systems disclosed herein can be administered once every three days, once every five days, once weekly, once every ten days, or once every two weeks. The administration frequency is timed to coincide with the designed gastric residence period of the gastric residence system which is administered, so that at about the same time that a gastric residence system passes out of the stomach after its residence period, a new gastric residence system is administered.

### Dissolution Profile, Bioavailability and Pharmacokinetics for Gastric Residence Systems

Dissolution: The gastric residence systems described herein provide a steady release of an agent or a pharmaceutically acceptable salt thereof over an extended period of time. The systems are designed to release a therapeutically effective amount of an agent or salt thereof over the period of residence in the stomach. The release of agent (or salt thereof) can be measured in vitro or in vivo to establish the dissolution profile (elution profile, release rate) of the agent (or salt thereof) from a given residence system in a specific environment. The dissolution profile can be specified as a percentage of the original amount of agent (or salt thereof) present in the system which elutes from the system over a given time period.

Thus, in some embodiments, the agent (or salt thereof) contained in a gastric residence system can have a dissolution profile of 10-20% release between zero hours and 24 hours in a given environment. That is, over the 24-hour period after initial introduction of the gastric residence system into the environment of interest, 10-20% of the initial agent (or salt thereof) contained in the system elutes from the system.

The environment of interest can be 1) the stomach of a patient (that is, an in vivo environment), or 2) simulated gastric fluid (that is, an in vitro environment).

The gastric residence systems disclosed herein provide for high bioavailability of the agent (or salt thereof) as measured by AUC_{inf} after administration of the systems, relative to the bioavailability of a conventional oral formulation of the agent (or salt thereof). The systems also provide for maintenance of a substantially constant plasma level of the agent (or salt thereof).

Parameters of interest for release include the linearity of release over the residence period of the gastric residence systems, the standard deviation of release over the residence period (which is related to linearity of release; a standard deviation of zero indicates that release is linear over the entire residence period), the release over the initial six hours of residence (that is, burst release upon initial administration), and total release of agent (or salt thereof) over the residence period. A preferable residence period is seven days, although other periods, such as two, three, four, five, six, eight, nine, ten, 11, 12, 13, or 14 days can be useful.

Linearity of agent (or salt thereof) release over the residence period refers to the amount released during each 24-hour period of residence. For a seven-day period of residence, it is desirable that about the amount of agent (or salt thereof) is released each day, i.e., that linearity of agent (or salt thereof) release is maximized. This will minimize the standard deviation of daily agent or agent salt release over the residence period. In some embodiments, the gastric release systems have a variation (or a standard deviation) for daily agent (or salt thereof) release of less than about 100%, less than about 90%, less than about 80%, less than about 70%, less than about 60%, less than about 50%, less than about 40%, less than about 30%, less than about 25%, less than about 20%, less than about 15%, less than about 10%, or less than about 5%, over the period of residence. In some embodiments, the period of residence can be about three days, about seven days, about ten days, or about two weeks.

Minimization of burst release, that is, release over the initial period of residence (such as six hours, twelve hours, or 24 hours after administration of a gastric residence system) is desirable in order to maintain a predictable and steady release profile. If T is the total agent (or salt thereof) release over the residence period (in units of mass), and D is the number of days of the residence period, then completely linear release would mean that about T/D mass of agent (or salt thereof) is released per day. If the period over which burst release is measured is the first six hours, then a linear release profile will result in 0.25×T/D mass of agent (or salt thereof) released during the first six hours. In percentage terms of the total amount of agent (or salt thereof) released over the residence period of D days, linear release would be about 100/D % of agent (or salt thereof) per day, and a linear release over the first six hours would be 25/D %. (Note that 100% in this context indicates the total amount of agent (or salt thereof) released, regardless of how much agent (or salt thereof) is contained in the initial formulation.) Thus, for a seven day residence period, linear release over the first six hours would be about 3.6% of the total amount of agent (or salt thereof) released over the seven-day period.

In some embodiments, during the initial six hours of residence after administration the gastric residence systems release about 0.2 to about 2 times T/D of the total mass of agent (or salt thereof) T released over the residence period of D days, or about 0.2 to about 1.75 times T/D of the total mass of agent (or salt thereof) T released over the residence period of D days, or about 0.2 to about 1.5 times T/D of the total mass of agent (or salt thereof) T released over the residence period of D days, or about 0.2 to about 1.25 times T/D of the total mass of agent (or salt thereof) T released over the residence period of D days, or about 0.2 to about 1 times T/D of the total mass of agent (or salt thereof) T released over the residence period of D days, or about 0.2 to about 0.8 times T/D of the total mass of agent (or salt thereof) T released over the residence period of D days, or about 0.2 to about 0.75 times T/D, or about 0.2 to about 0.7 times T/D, or about 0.2 to about 0.6 times T/D, or about 0.2 to about 0.5 times T/D, or about 0.2 to about 0.4 times T/D, or about 0.2 to about 0.3 times T/D, or about 0.25 to about 2 times T/D, or about 0.3 to about 2 times T/D, or about 0.4 to about 2 times T/D, or about 0.5 to about 2 times T/D, or about 0.6 to about 2 times T/D, or about 0.7 to about 2 times T/D, or about 0.25 to about 1.5 times T/D, or about 0.3 to about 1.5 times T/D, or about 0.4 to about 1.5 times T/D, or about 0.5 to about 1.5 times T/D, or about 0.6 to about 1.5 times T/D, or about 0.7 to about 1.5 times T/D, or about 0.25 to about 1.25 times T/D, or about 0.3 to about 1.25 times T/D, or about 0.4 to about 1.25 times T/D, or about 0.5 to about 1.25 times T/D, or about 0.6 to about 1.25 times T/D, or about 0.7 to about 1.25 times T/D, or about 0.25 to about 1 times T/D, or about 0.3 to about 1 times T/D, or about 0.4 to about 1 times T/D, or about 0.5 to about 1 times T/D, or about 0.6 to about 1 times T/D, or about 0.7 to about 1 times T/D, or about 0.25 times T/D, or about 0.25 to about 0.8 times T/D, or about 0.3 to about 0.8 times T/D, or about 0.4 to about 0.8 times T/D, or about 0.5 to about 0.8 times T/D, or about 0.6 to about 0.8 times T/D, or about 0.7 to about 0.8 times T/D, or about 0.8 times T/D, about 1 times T/D, about 1.25 times T/D, about 1.5 times T/D, or about 2 times T/D.

In some embodiment of the gastric residence systems, during the initial six hours of residence after administration the gastric residence systems release about 2% to about 10% of the total mass of agent (or salt thereof) released over the residence period, or about 3% to about 10%, or about 4% to about 10%, or about 5% to about 10%, or about 6% to about 10%, or about 7% to about 10%, or about 8% to about 10%, or about 9% to about 10%, or about 2% to about 9%, or about 2% to about 8%, or about 2% to about 7%, or about 2% to about 6%, or about 2% to about 5%, or about 2% to about 4%, or about 2% to about 3%.

In some embodiments of the gastric residence systems, where the gastric residence systems have a residence period of about seven days, during the initial six hours of residence after administration the gastric residence systems release about 2% to about 10% of the total mass of agent (or salt thereof) released over the residence period of seven days, or about 3% to about 10%, or about 4% to about 10%, or about 5% to about 10%, or about 6% to about 10%, or about 7% to about 10%, or about 8% to about 10%, or about 9% to about 10%, or about 2% to about 9%, or about 2% to about 8%, or about 2% to about 7%, or about 2% to about 6%, or about 2% to about 5%, or about 2% to about 4%, or about 2% to about 3%.

In some embodiments, during the initial 24 hours of residence after administration, the gastric residence systems release about 10% to about 35% of the total mass of agent (or salt thereof) released over the residence period, or about 10% to about 30%, or about 10% to about 25%, or about 10% to about 20%, or about 10% to about 15%, or about 15% to about 35%, or about 15% to about 35%, or about 15% to about 30%, or about 20% to about 30%, or about 25% to about 35%, or about 25% to about 30%, or about 30% to about 35%.

In some embodiments, where the gastric residence systems have a residence period of about seven days, during the initial 24 hours of residence after administration the gastric residence systems release about 10% to about 35% of the total mass of agent (or salt thereof) released over the residence period of seven days, or about 10% to about 30%, or about 10% to about 25%, or about 10% to about 20%, or about 10% to about 15%, or about 15% to about 35%, or about 15% to about 35%, or about 15% to about 30%, or about 20% to about 30%, or about 25% to about 35%, or about 25% to about 30%, or about 30% to about 35%.

### Kits and Articles of Manufacture

Also provided herein are kits for treatment of patients with the gastric residence systems as disclosed herein. The kit may contain, for example, a sufficient number of gastric residence systems for periodic administration to a patient over a desired total treatment time period. If the total treatment time in days is (T-total), and the gastric residence systems have a residence time of (D-days), then the kit will contain a number of gastric residence systems equal to ((T-total) divided by (D-days)) (rounded to an integral number), for administration every D-days. Alternatively, if the total treatment time in days is (T-total), and the gastric residence systems have an effective release period of (E-days), then the kit will contain a number of gastric residence systems equal to ((T-total) divided by (E-days)) (rounded to an integral number), for administration every E-days. The kit may contain, for example, several gastric residence systems in containers (where the containers may be capsules) and may optionally also contain printed or computer readable instructions for dosing regimens, duration of treatment, or other information pertinent to the use of the gastric residence systems and/or the agent or drug contained in the gastric residence systems. For example, if the total treatment period prescribed for the patient is one year, and the gastric residence system has a residence time of one week or an effective release period of one week, the kit may contain 52 capsules, each capsule containing one gastric residence system, with instructions to swallow one capsule once a week on the same day (e.g., every Saturday).

Articles of manufacture, comprising a sufficient number of gastric residence systems for periodic administration to a patient over a desired total treatment time period, and optionally comprising instructions for dosing regimens, duration of treatment, or other information pertinent to the use of the gastric residence systems and/or the agent or drug contained in the gastric residence systems, are also included in the disclosure. The articles of manufacture may be supplied in appropriate packaging, such as dispensers, trays, or other packaging that assists the patient in administration of the gastric residence systems at the prescribed interval.

### EXAMPLES

The technology as disclosed herein is further illustrated by the following non-limiting examples.

### Example 1: FaSSGF Preparation

FaSSGF was prepared as follows, according to the manufacturer's instructions (biorelevant.com). 975 mL deionized water and 25 mL of 1N hydrochloric acid were mixed in a 1 L glass media bottle. The pH was adjusted to 1.6 using 1N HCl or NaOH as needed. 2.0 grams of NaCl was added and mixed in. Just before use, 60 mg of Biorelevant powder was mixed into the solution. The composition of FaSSGF is taurocholate (0.08 mM), phospholipids (0.02 mM), sodium (34 mM), chloride (59 mM).

### Example 2: Dip Coating Provides Release Rate Control for High Drug Load Formulations

### Drug Arm Formulation Preparation:

All non-PCL powders were blended and wet granulated with water. The dried granules were then blended with PCL powder and compounding extrusion was performed using a twin screw extruder. Profile extrusion was subsequently performed using a twin screw extruder. DNP34 and M116 arm formulations as described in Table 1 were used for dip coating experiments.

TABLE 1

| Name | Composition | Function |
|---|---|---|
| MEM116 | 45% MEM, 41.9% PCL, 10% PDL20, 2% | memantine |
| | P407, 0.5% Vit. E Succinate, 0.5% SiO2, | formulation |
| | 0.1% pigment | |
| MEM122 | 50% MEM, 43.97% PCL, 5% Kollidon SR, | memantine |
| | 0.5% Vit E Succinate, 0.5% SiO2, 0.03% | formulation |
| | pigment | |
| DNP34 | 40% DNP, 44% PCL, 10% PDL20, 5% P407, | donepezil |
| | 0.5% Vit. E Succinate, 0.5% SiO2 | formulation |
| MD01 | 35% MEM, 14.5% DNP, 43.97% PCL, 5% | memantine + |
| | Kollidon SR, 0.5% Vit E Succinate, 0.5% | donepezil |
| | SiO2, 0.03% pigment | formulation |
| RSP49 | 35% RSP, 55.9% PCL, 5% VA64, 3% P407, | risperidone |
| | 0.5% Vit. E Succinate, 0.5% SiO2, 0.1% | formulation |
| | pigment | |
| D138 | 20% daoagliflozin, 33.99% PCL, 30% VA64, | dapagliflozin |
| | 10% PDL20, 5% Sorbitan monostearate | formulation |
| | (Span60), 0.5% Vit. E Succinate, 0.5% | |
| | Colloidal silicon dioxide (M5P), 0.01% | |
| | pigment | |

Dip coating: Dip coating solutions were prepared as follows: the solid contents of each coating solution were weighed directly into a glass vial. Solvent was added to reach the appropriate solids content (% w/v). The solutions were stirred at 65° C. and 300 rpm until solids were solubilized or uniformly suspended. Exemplary compositions of coating solutions are listed in Table 2. All dip coating formulations were prepared in ethyl acetate either as a solution or as a stable suspension (for coating formulations with insoluble ingredients such as porogens). All solutions and suspensions were prepared at 8% w/v solid content, except for solutions containing PEG 10K, which were prepared at 5% w/v solid content and suspensions containing K90F at 6-8 w/v. Drug arms were gripped with forceps, completely submerged in the coating solution, and immediately removed. Coated arms were dried in a fume hood overnight. In all dip-coating experiments, the PDL used was Corbion Purasorb PDL20, a PDL having 2.0 dl/g intrinsic viscosity (range 1.6 dl/g to 2.4 dl/g). In all dip-coating experiments, the PDLG used was Corbion Purasorb PDLG 5004A, an acid terminated copolymer of DL-lactide and glycolide (50/50 molar ratio) having an inherent viscosity midpoint of 0.4 dl/g. For dip coating, PCL HMW was 80 kD or 2.07 dL/g in CHCl₃ and PCL LMW was 14 kD. PDL-PCL2575 used was Lactel® 25:75 poly(DL-lactide-co-ε-caprolactone) with inherent viscosity 0.70-0.90 dl/g, while PDL-PCL8020 was Lactel® 80:20 poly(DL-lactide-co-ε-caprolactone) with inherent viscosity 0.70-0.90 dl/g.

TABLE 2
Coating formulations for dip coating.

| Formulation No. | Coating Formulation |
|---|---|
| 1 | PDL |
| 2 | PCL HMW |
| 3 | 1:1 PCL HMW:PCL LMW |
| 4 | 3:1, PDL:VA64 |
| 5 | 3:1, PCL HMW:K90F |
| 6 | 9:1, PDL:PEG1 |
| 7 | 9:1, PDL:L31 |
| 8 | 27:2:1, PDL:PEG1:PPG |
| 9 | 9:1, PCL HMW:PG |
| 10 | 9:1, PCL HMW:PPG |
| 11 | 9:1, PCL HMW:L-31 |
| 12 | 9:1, PCL HMW:F-108 |
| 13 | 27:2:1, PCL HMW:PEG1:PPG |
| 14 | 9:1, PCL HMW:PCL triol |
| 15 | 9:1, PDL:PG |
| 16 | 9:1, PDL:PPG |
| 17 | 9:1, PDL:L-31 |
| 18 | 9:1, PDL:F-108 |
| 19 | 27:2:1, PDL:PEG1:PPG |
| 20 | 4:1, PDL:K90F |
| 21 | 4:1, PDL:PVPP |
| 22 | 9:27:4 PDL:PCL:PEG |
| 23 | 36:9:5 PDL:PCL:PEG |
| 24 | 9:1, PDL-PCL2575:PEG1 |
| 25 | 9:1, PDL-PCL8020:PEG1 |
| 26 | PDLG 5004A |
| 27 | 3:1, PDLG:VA64 |
| 28 | 3:1, PCL HMW:PVPP |
| 29 | 4:1, PDL:K90F |
| 30 | 3:1, PDL:PVPP |
| 31 | 3:1, PDL-PCL2575:K90F |
| 32 | 3:1, PDL-PCL2575:PVPP |
| 33 | 3:1, PDL-PCL8020:K90F |
| 34 | 3:1, PDL-PCL8020:PVPP |
| 35 | PVAc |
| 36 | PDL-PCL2575 |
| 37 | PDL-PCL8020 |
| 38 | 3:1, PVAc:VA64 |
| 39 | 3:1, PDL-PCL2575:VA64 |
| 40 | 3:1, PDL-PCL8020:VA64 |
| 41 | 2:1, PCL HMW:PCL LMW |
| 42 | 1:2, PCL HMW:PCL LMW |
| 43 | 9:1, PCL HMW:PEG10 |
| 44 | 9:1, PDL:PEG1 |
| 45 | 9:1, PDL:PEG10 |
| 46 | 9:1, PCL HMW:PEG1 |
| 47 | 9:1, PVAc:PEG1 |
| 48 | 9:1, PVAc:PEG10 |
| 49 | 9:1, PDL-PCL2575:PEG10 |
| 50 | 9:1, PDL-PCL8020:PPEG10 |
| 64 | 9:1, PCL HMW:mannitol |

In Vitro Release: Each formulation was applied to DN34 drug arms and evaluated for release in fasted state simulated gastric fluid (FaSSGF) for seven days. Release rates were evaluated using the procedures provided below for donepezil.

### Example 3: Pan Coating Provides Release Rate Control for High and Low Drug Load Formulations

Drug Arm Formulation Preparation: The underlined drugs as indicated in Table 1 were respectively blended into drug-loaded arms using one of the following procedures.

Procedure #1: All non-API powders were bag blended by hand until a visually uniform mixture was achieved. API was added and the mixture bag blended further until a visually uniform mixture was again achieved. Compounding extrusion was performed using a twin screw extruder at 140° C. Profile extrusion was performed using a twin screw extruder and a temperature gradient of 120° C. to 100° C. to maintain the desired shape.

Procedure #2: All non-API powders were bag blended by hand until a visually uniform mixture was achieved. API was added and the mixture bag blended further until a visually uniform mixture was again achieved. Compounding extrusion was performed using a twin screw extruder and temperature gradient of 115-130° C. Profile extrusion was performed using a single screw extruder and a temperature gradient of 50-80° C.

Procedure #3: All non-PCL powders were blended and wet granulated with water. The dried granules were then blended with PCL powder and compounding extrusion was performed using a twin screw extruder. Profile extrusion was subsequently performed using a twin screw extruder.

Procedure #4: Each API was granulated independently with all other non-PCL powders. The powder mixes were blended wet granulated with water. The dried granules containing memantine, dried granules containing donepezil, and PCL powder were then blended and compounding extrusion was performed using a twin screw extruder. Profile extrusion was subsequently performed using a single screw extruder. Arm formulations used are listed in Table 1.

Exemplary compositions of pan coating solutions are listed in Table 3. Pan-coating procedures were carried out as described below.

### Poly-Lactide-Based Films

Solutions of poly-lactide-based polymers were prepared in neat and dry acetone with solid concentrations of 1.5-3.3% w/v. Solutions were prepared in one of two methods described below, with each method demonstrating comparable performance in both the film coating process and in drug release.

Method 1: PDL20 was removed from −20° C. freezer and equilibrated to room temperature for at least 2 hours. A stir bar and glass bottle for solution preparation were triple rinsed with acetone. The wash solvent was decanted and evaporated. Half of the desired mass of acetone was placed in the glass bottle with the stir bar and set to stir at 180-200 RPM at room temperature. The entire mass of PDL20 required in formulation was slowly added to the stirring acetone. The glass bottle was then capped, sealed with parafilm, and left to stir overnight. Subsequently, the solution was allowed to settle. If any particulates were observed, the solution was decanted and re-weighed. The additional desired mass of acetone was then added to the solution. PDLG5002A was removed from −20° C. freezer and equilibrated to room temperature for at least 2 hours. The entire mass of PDLG5002A required in formulation was slowly added to the stirring solution containing PDL20 and acetone. The solution was then set to stir at room temperature at 180-200 RPM for at least 30 minutes. Magnesium stearate was added in one portion to the stirring solution and allowed to stir at 180-200 RPM under room temperature for at least 10 minutes to achieve a homogenous dispersion. The suspension was weighed and filled to mass with acetone if needed.

Method 2: PDL20 was removed from −20° C. freezer and equilibrated to room temperature for at least 2 hours. A glass bottle and impeller for solution preparation were triple rinsed with acetone. The wash solvent was decanted and evaporated. The desired mass of acetone was placed in the glass bottle and set to stir at 500 RPM at room temperature. The entire mass of PDL20 required in formulation was slowly added to the stirring acetone. The glass bottle was then capped, sealed with parafilm, and left to stir for at least 2 hours. Subsequently, the solution was allowed to settle. If any particulates were observed, the solution was decanted, re-weighed, and filled to mass with acetone if needed. PDLG5002A was removed from −20° C. freezer and equilibrated to room temperature for at least 2 hours. The entire mass of PDLG5002A required in formulation was slowly added to the stirring solution containing PDL20 and acetone. The solution was allowed to stir at 500 RPM under room temperature for at least an additional 30 minutes. Magnesium stearate was added into one portion of the solution with continued stirring. The resulting suspension was stirred for at least 5 minutes to achieve a homogenous dispersion. The suspension was then weighed and filled to mass with acetone if needed.

Procedures similar to Method 1 and Method 2 were used for preparation of PDL20 coating solutions using other additional polymers instead of PDLG5002A. Separately, a mixture of placebo arms and drug arms totaling 480 g was prepared. The quantity of drug-containing arms was approximately 1% to 25% by weight.

The coating solution, maintained under agitation with a stir bar during spraying, was then applied to the mixture of placebo and drug loaded arms using a LDCS Hi-Coater pharmaceutical pan coater with manufacturer-supplied spray nozzle (Freund-Vector, Marion, Iowa, USA). The following parameters were used: inlet air temperature (48° C.), exhaust air temperature (36-38° C.), airflow (50 CFM), pan run speed (22 RPM), atomization pressure (20 PSI), pattern pressure (18 PSI). A-Pharm-Line acetone-resistant tubing was used with the built-in peristaltic pump and was pre-washed with 50 g of neat and dry acetone. The mixture of placebo and drug arms was then loaded into the pan. Solution was applied in 12 minute intervals followed by 5 minutes of tumbling. This procedure was repeated until a desired mass gain of approximately 1-6% (w/w) was achieved. Mass gain was determined based on the amount of solution sprayed. After the desired quantity of solution was sprayed, arms were dried for at least 2 hours at ambient condition to drive off any residual acetone. After evaporation, arms were stored sealed with desiccant until use in drug release studies.

In all pan-coating experiments, the PDL used was Corbion Purasorb PDL20, a PDL having 2.0 dl/g intrinsic viscosity (range 1.6 dl/g to 2.4 dl/g). In all pan-coating experiments, the PDLG used was either Corbion Purasorb PDLG 5004A (an acid terminated copolymer of DL-lactide and glycolide in 50/50 molar ratio, having an inherent viscosity midpoint of 0.4 dl/g), or Corbion Purasorb PDLG 5002A (an acid terminated copolymer of DL-lactide and glycolide in a 50/50 molar ratio, having an inherent viscosity midpoint of 0.2 dl/g).

### Polycaprolactone-Based Films

Solutions containing polycaprolactone-based polymers were prepared in neat and dry ethyl acetate with solid concentration of 3.3% w/v.

A glass bottle and impeller for solution preparation were triple rinsed with ethyl acetate. The wash solvent was decanted and evaporated. The desired mass of ethyl acetate was weighed in the glass bottle. The solid PCL was weighed and added to the glass bottle containing ethyl acetate. The bottle was then placed on a hot plate set at approximately 45° C. and set to stir at between 500-550 RPM using an overhead stirrer (IKA Works Inc., Wilmington, N.C., USA). The bottle was then capped and left to stir for approximately 30 minutes. Once PCL was fully dissolved, Kollidon VA64 was added to it with continued stirring. Once the VA64 was solubilized, heating was stopped, and the hot plate was removed. Magnesium stearate was added, and the suspension was continually stirred until cooled to room temperature. Procedures similar to this method were used for preparation of PCL coating solutions using other ethyl acetate-soluble ingredients instead of VA64. Separately, a mixture of placebo arms and drug arms totaling approximately 485 g was prepared. The quantity of drug-containing arms was approximately 1% to 25% by weight.

The coating solution, maintained under agitation with a stir bar during spraying, was then applied to mixture of placebo and drug loaded arms using a LDCS Hi-Coater pharmaceutical pan coater with manufacturer-supplied spray nozzle (Freund-Vector, Marion, Iowa, USA). The following parameters were used: inlet air temperature (50° C.), exhaust air temperature (40-42° C.), airflow (50 CFM), pan run speed (22 RPM), atomization pressure (20-22 PSI), pattern pressure (18-20 PSI). Ethyl acetate-resistant tubing was used with the built-in peristaltic pump and was pre-washed with approximately 50 ml of neat solvent. The mixture of placebo and drug arms were then loaded into the pan. Solution was applied in 5-minute intervals followed by 3 minutes of tumbling. This procedure was repeated until a desired mass gain of approximately 1-6% (w/w) was achieved. Mass gain was determined based on solution sprayed on the placebo and drug arms in the pan. After coating, arms were stored at ambient conditions until used in drug release studies. For PCL used in pan coating, high molecular weight PCL (PCL HMW) had intrinsic viscosity of 1.7 dl/g, while low molecular weight PCL (PCL LMW) had intrinsic viscosity less than or equal to 0.8 dl/g, most typically less than 0.4 dl/g.

TABLE 3
Coating formulations for pan coating.

| Formulation | | Coating | Coating Solution |
|---|---|---|---|
| Code | Coating Formulation | Solvent | Concentration (% w/v) |
| 51 | 9:1, PDL:PEG1; 2% Mg stearate by | Ethyl acetate | 2.6 |
| | weight of solids | | |
| 52 | 1:1, PDL:PDLG; 2% Mg stearate | Acetone | 1.5 |
| | by weight of solids | | |
| 53 | 3:1, PCL HMW:VA64; 2% Mg | Ethyl Acetate | 3.3 |
| | stearate by weight of solids | | |
| 54 | PDLG5004; 2% Mg stearate by | Acetone | 1.5 |
| | weight of solids | | |
| 55 | 1:1, PDL:PDLG; 2% Mg stearate | Acetone | 1.5 |
| | by weight of solids | | |
| 56 | 9:1, PCL HMW:P407; 2% Mg | Ethyl Acetate | 3.3 |
| | stearate by weight of solids | | |
| 57 | PCL midMW; 2% Mg stearate by | Ethyl Acetate | 3.3 |
| | weight of solids | | |
| 58 | 1:3, PCL HMW:PCL LMW; 2% | Ethyl Acetate | 3.3 |
| | Mg stearate by weight of solids | | |
| 59 | 4:6, PCL HMW:PCL LMW; 2% | Ethyl Acetate | 3.3 |
| | Mg stearate by weight of solids | | |
| 60 | 1:1, PCL HMW:PCL LMW; 2% | Ethyl Acetate | 3.3 |
| | Mg stearate by weight of solids | | |
| 61 | 3:1, PCL HMW:PCL LMW; 2% | Ethyl Acetate | 3.3 |
| | Mg stearate by weight of solids | | |
| 62 | 85:15, PCL HMW:PCL LMW; 2% | Ethyl Acetate | 3.3 |
| | Mg stearate by weight of solids | | |
| 63 | 9:1, PCL HMW:PCL LMW; 2% | Ethyl Acetate | 3.3 |
| | Mg stearate by weight of solids | | |
| 65 | 6:4, PCL HMW:PCL LMW; 2% | Ethyl Acetate | 3.3 |
| | Mg stearate by weight of solids | | |

### Example 4: In Vitro Drug Release Assay and Exposure to Welding Conditions for Pan-Coated or Dip-Coated Drug Arms

In Vitro release: In vitro release of drugs for coated drug arms was conducted as follows for the various drugs.

To measure dapagliflozin release, fasted state simulated gastric fluid (FaSSGF; biorelevant.com LTD, London, UK) was prepared per the manufacturer's instructions. Individual coated drug arms were placed in flat bottom 20 mL glass scintillation vials with 10 mL FaSSGF. Each vial was placed upright in an Innova43 shaking incubator (Eppendorf AG, Hamburg, Germany) at 200 RPM and 37° C. Drug content in the FaSSGF was analyzed by HPLC at least four times over at least seven days. Samples were stored for no more than 3 days at 4° C. prior to analysis. At each measurement time point, in order to maintain sink conditions, the entire volume of release media was replaced with fresh solution pre-equilibrated to 37° C.

To measure donepezil release, fasted state simulated gastric fluid (FaSSGF; biorelevant.com LTD, London, UK) was prepared per the manufacturer's instructions. Individual coated drug arms were placed in conical bottom 15 mL polypropylene tubes with 10 mL FaSSGF. Each tube was placed upright in an Innova43 shaking incubator (Eppendorf AG, Hamburg, Germany) at 200 RPM and 37° C. Drug content in the FaSSGF was analyzed by HPLC at least four times over at least seven days. Samples were stored for no more than 3 days at 4° C. prior to analysis. At each measurement time point, in order to maintain sink conditions, the entire volume of release media was replaced with fresh solution pre-equilibrated to 37° C.

To measure memantine release, fasted state simulated gastric fluid (FaSSGF; biorelevant.com LTD, London, UK) was prepared per the manufacturer's instructions. Individual coated drug arms were placed in in conical bottom 15 mL polypropylene tubes with 10 mL FaSSGF. Each tube was placed upright in an Innova43 shaking incubator (Eppendorf AG, Hamburg, Germany) at 200 RPM and 37° C. Drug content in the FaSSGF was analyzed by HPLC with pre-column derivatization at least four times over at least seven days. Samples were stored for no more than 3 days at 4° C. prior to analysis. At each measurement time point, in order to maintain sink conditions, the entire volume of release media was replaced with fresh solution pre-equilibrated to 37° C.

Thermal exposure: To test the effect of residence system assembly on the coating, drug-loaded arms were thermally exposed to the same process or a similar process used to assemble dosage forms and dosage form components (i.e., composite arms). Welding operations were performed using a custom fixture that enables control of weld temperature, applied pressure, and material alignment. In typical heat-assisted assembly, irradiation of drug-loaded arms reaches temperatures of approximately 60-160° C., most commonly below 120° C. In typical heat-assisted assembly, pressures of 15-60 psi are applied to one or both sides of an arm. Arms were exposed to IR and pressure either by a) using welding conditions identical to those used for preparation of a stellate system, by welding arms to a liquid silicone rubber (LSR) core, then cutting them from the stellate for in vitro release study or b) welding conditions identical to those used for preparation of composite arms (i.e., inactive-active-inactive segments), which are welding conditions highly similar to that used in preparation of stellate system. Alternatively, arms can be welded under the same conditions as to an LSR core, but using an aluminum core insert as a placeholder. These scenarios which are comparable to preparation of a stellate dosage form for animal or human dosing, where a drug arm is only partly exposed to IR. In scenario b) full arms can be exposed to IR and pressure without being attached to anything, which represents a “worst case” scenario where an entire arm is exposed to IR (which is not representative of stellate assembly).

After welding, all drug-loaded arms were stored at room temperature for at least overnight to facilitate complete re-crystallization before drug release was evaluated. In vitro release of drug was performed on single (“isolated”) arms in individual vials.

### Example 5: Effect of PC30 Coating on Drug Release Kinetics for Welded Gastric Residence System with Low Load Memantine/Donepezil Formulation (MD01)

To elucidate the effect of a candidate PCL-based coating on memantine and donepezil drug release in residence systems, drug arms for MD01 were prepared, pan-coated with PC30 (60:40 w/w, Corbion PC17:Corbion PC04+2% Mg stearate by weight of solids) using procedures as described in Example 3, subjected to IR exposure resembling typical assembly, and tested for in vitro drug release as described below. Corbion PC17 is a high molecular weight PCL with an inherent viscosity midpoint of 1.7 dl/g (range 1.5-1.9 dl/g), while Corbion PC04 is a low molecular weight PCL with an inherent viscosity midpoint of 0.4 dl/g (range 0.35 dl/g to 0.43 dl/g).

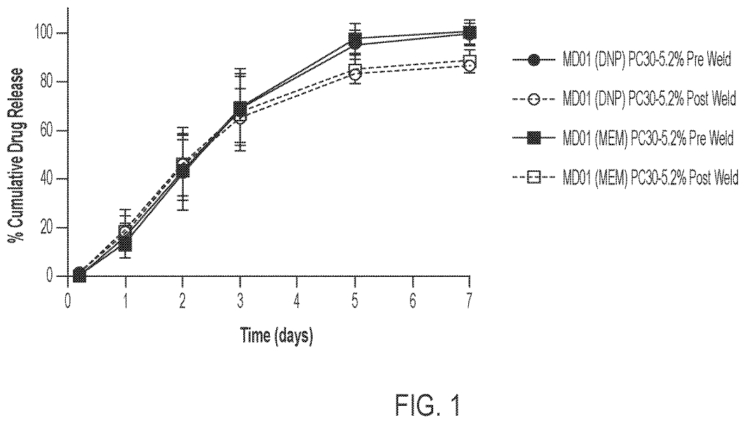

In Vitro Release: MD01 was evaluated for release in fasted state simulated gastric fluid (FaSSGF) for seven days. Drug arms weighing approximately 25-150 mg were generally used to evaluate in vitro release, most typically arms weighing approximately 100 mg. Carrier polymer-agent formulation was processed into drug arms, pan-coated with PC30, and evaluated for drug release kinetics before and after exposure to welding conditions (IR exposure to 4 to 7 mm out of the 14 mm drug arm) according to Example 4. Coat weight gain was approximately 5.2% for PC30-coated arms. The cumulative drug release was plotted and shown in FIG. 1.

As shown in FIG. 1, release of both memantine and donepezil could be modulated and controlled by use of an appropriate release-rate modulating film, as demonstrated by the linear release rate achieved over 7 days by pan-coating MD01 drug arms with PC30 coating solution in ethyl acetate. FIG. 1 further showed that exposure of the coated arms to welding conditions did not affect the linear drug release rate over at least 7 days, indicating that the release modulation afforded by PC30 coating formulation would not be adversely affected by the welding process used in gastric residence system assembly.

### Example 6: Effect of PC25 and PC26 Coating on Drug Release Kinetics for Welded Gastric Residence System with Low Load Donepezil Formulation (DNP34)

To elucidate the effect of candidate PCL-based coatings on donepezil drug release in residence systems, drug arms for DNP34 were prepared, pan-coated with either PC25 (50:50 w/w, Corbion PC17: Corbion PC02; +2% Mg stearate by weight of solids), PC26 (75:25 w/w, Corbion PC17: Corbion PC04; +2% Mg stearate by weight of solids), or control coating PC17 (75:25 w/w, Corbion PC17: VA64; +2% Mg stearate by weight of solids) as described in Example 3, subjected to IR exposure resembling typical assembly, and tested for in vitro drug release as described below. Corbion PC17 is a high molecular weight PCL with an intrinsic viscosity midpoint of 1.7 dl/g, while Corbion PC02 and Corbion PC04 are low molecular weight PCL with intrinsic viscosity midpoints of 0.2 dl/g (PC02) and 0.4 dl/g (PC04).

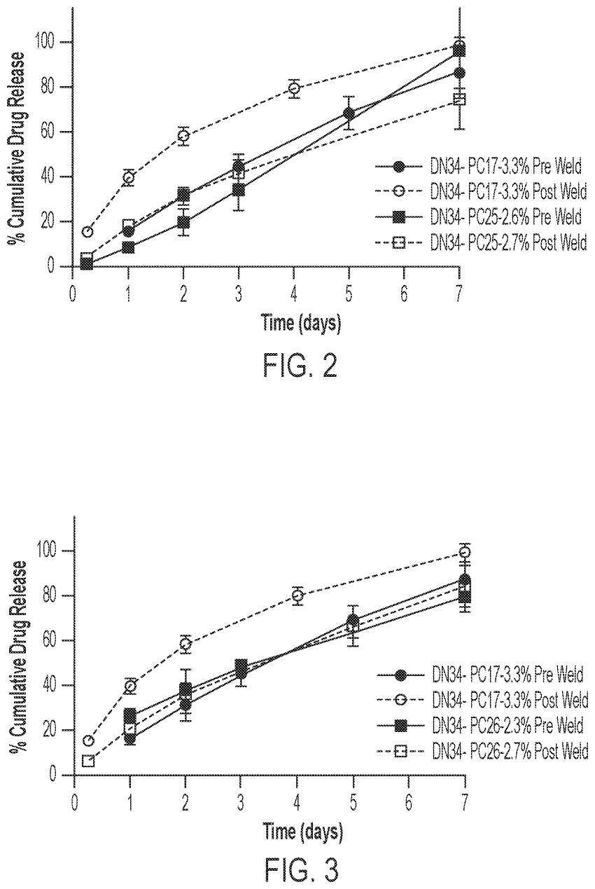

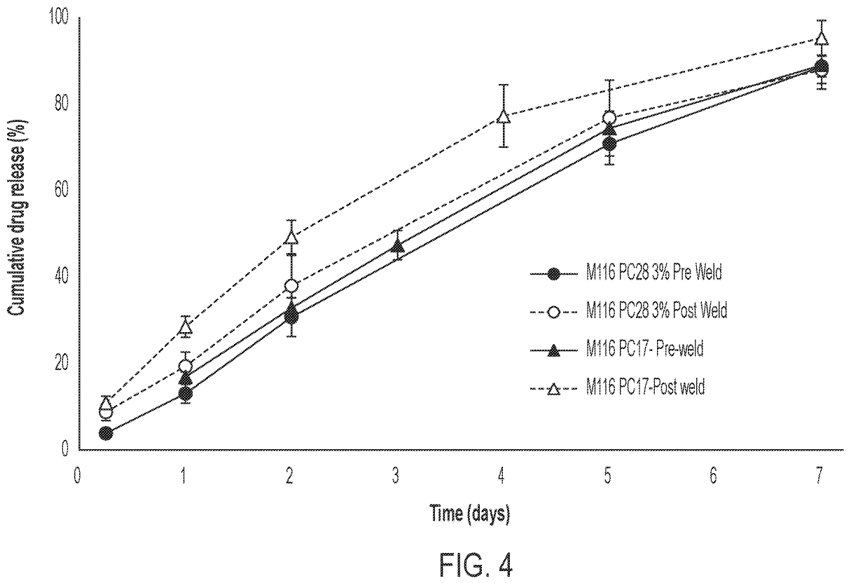

In Vitro Release: DNP34 was evaluated for release in fasted state simulated gastric fluid (FaSSGF) for seven days. Drug arms within a general range of approximately 25-150 mg, typically weighing approximately 100 mg, were used to evaluate in vitro release. Drug arms were pan-coated with PC25, PC26, or PC17 and evaluated for drug release kinetics before and after exposure to welding conditions (IR exposure to 4 to 7 mm out of the 14 mm drug arm) according to Example 4. The coat weight gain was approximately 2.7% for PC25, 2.5% for PC26, and 3.3% for PC17. The cumulative drug release with PC25 or P26 coating was compared to that with PC17 coating, and shown in FIGS. 2 and 3, respectively.

As shown in FIGS. 2 and 3, by pan-coating DNP34 drug arms with PC17 coating solutions in ethyl acetate, linear release of donepezil could be achieved over 7 days. However, release kinetics shifted significantly when PC17-coated DNP34 drug arms were subjected to welding conditions. In contrast, release of donepezil could be modulated and controlled by use of an appropriate release-rate modulating film, as demonstrated by the linear release rate achieved over 7 days by pan-coating DNP34 drug arms with PC25 or PC26 coating solutions in ethyl acetate (FIG. 2, 3 respectively), where exposure of the coated arms to welding conditions did not affect the linear drug release rate over at least 7 days, indicating that the release modulation afforded by PC25 or PC26 coating formulations would not be adversely affected by the welding process used in gastric residence system assembly (FIG. 2, 3 respectively).

The PC17 coating contains the pore-forming agent VA64 (copovidone; vinylpyrrolidone-vinyl acetate copolymer), and is believed to form non-homogeneous coatings. The non-homogeneous coatings are disrupted during heat-assisted assembly or procedures similar to heat exposure during heat-assisted assembly, leading to large differences between the release rate from coated drug arms before heat exposure as compared to coated drug arms after heat exposure. These results demonstrate the advantage of using homogeneous release-rate modulating films, without porogens or other elements that result in a non-homogeneous coating.

### Example 7: Effect of PC28 Coating on Drug Release Kinetics for Welded Gastric Residence System with Low Load Memantine Formulation (MEM116)

To elucidate the effect of a candidate PCL-based coatings on memantine drug release in residence systems, drug arms for MEM116 were prepared, pan-coated with either PC28 (50:50 w/w, Corbion PC17: Corbion PC04; +2% Mg stearate by weight of solids), or control coating PC17 (75:25 w/w, Corbion PC17: VA64; +2% Mg stearate by weight of solids) as described in Example 3, subjected to IR exposure resembling typical assembly, and tested for in vitro drug release as described below. Corbion PC17 is a high molecular weight PCL while Corbion PC04 is a low molecular weight PCL.

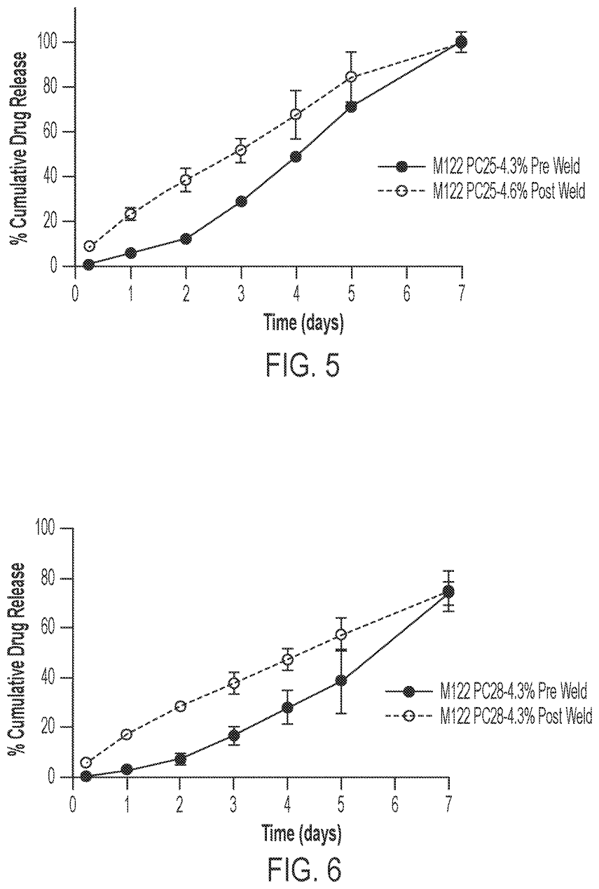

In Vitro Release: MEM116 was evaluated for release in fasted state simulated gastric fluid (FaSSGF) for seven days. Drug arms within a general range of approximately 25-150 mg, typically weighing approximately 100 mg, were used to evaluate in vitro release. Drug arms were pan-coated with PC17 or PC28, and evaluated for drug release kinetics before and after exposure to welding conditions (IR exposure to 4 to 7 mm out of the 14 mm drug arm) according to Example 4. The coat weight gain was 3% for PC28. The cumulative drug release with PC28 coating was compared to that with PC17 coating, and shown in FIG. 4.

As shown in FIG. 4, by pan-coating MEM116 drug arms with PC17 coating solutions in ethyl acetate, linear release of memantine could be achieved over 7 days. However, the release kinetics shifted significantly when PC17-coated MEM116 drug arms were subjected to welding conditions. In contrast, release of memantine could be modulated and controlled by use of an appropriate release-rate modulating film, as demonstrated by the linear release rate achieved over 7 days by pan-coating MEM116 drug arms with PC28 coating solutions in ethyl acetate (FIG. 4), where exposure of the coated arms to welding conditions had very little effect on the linear drug release rate over at least 7 days, indicating that the release modulation afforded by PC28 coating formulation would not be adversely affected by the welding process used in gastric residence system assembly (FIG. 4).

As in the previous example, the PC17 coating contains the pore-forming agent VA64 (copovidone; vinylpyrrolidone-vinyl acetate copolymer), and is believed to form non-homogeneous coatings. The non-homogeneous coatings are disrupted during heat-assisted assembly or procedures similar to heat exposure during heat-assisted assembly, leading to large differences between the release rate from coated drug arms before heat exposure as compared to coated drug arms after heat exposure. These results demonstrate the advantage of using homogeneous release-rate modulating films, without porogens or other elements that result in a non-homogeneous coating.

### Example 8: Effect of PC25 and PC28 Coating on Drug Release Kinetics for Welded Gastric Residence System with High Load Memantine Formulation (MEM122)

To elucidate the effect of a candidate PCL-based coating on memantine and donepezil drug release in residence systems, drug arms for MEM122 were prepared, pan-coated with either PC25 (50:50 w/w, Corbion PC17: Corbion PC02; +2% Mg stearate by weight of solids) or PC28 (50:50 w/w, Corbion PC17: Corbion PC04; +2% Mg stearate by weight of solids) using procedures as described in Example 3, subjected to IR exposure resembling typical assembly, and tested for in vitro drug release as described below.

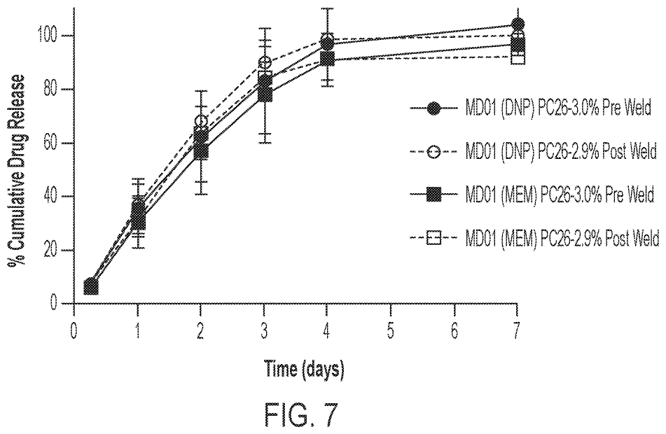

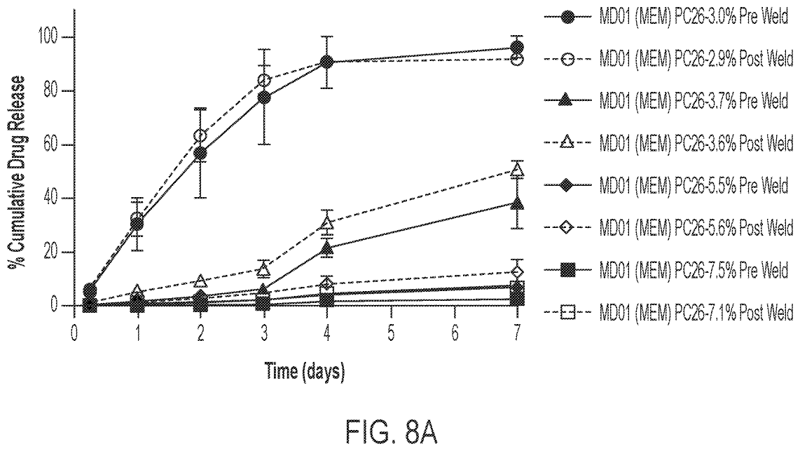

In Vitro Release: MEM122 was evaluated for release in fasted state simulated gastric fluid (FaSSGF) for seven days. Drug arms within a general range of approximately 25-150 mg, typically weighing approximately 100 mg, were used to evaluate in vitro release. Drug arms were pan-coated with PC25 or PC28, and evaluated for drug release kinetics before and after exposure to welding conditions (IR exposure to 4 to 7 mm out of the 14 mm drug arm) according to Example 4. Coating weight gain was approximately 4.5% for both PC25 and PC28. The cumulative drug release was plotted and shown in FIGS. 5 and 6, respectively.

As shown in FIGS. 5 and 6, release of memantine could be modulated and controlled by use of an appropriate release-rate modulating film.

### Example 9: Effect of PC26 Coating on Drug Release Kinetics for Welded Gastric Residence System with Low Load Memantine/Donepezil Formulation (MD01)

To elucidate the effect of a candidate PCL-based coatings on memantine and donepezil drug release in residence systems, drug arms for MD01 were prepared, pan-coated with PC26 (75:25 w/w, Corbion PC17: Corbion PC04; +2% Mg stearate by weight of solids) using procedures as described in Example 3, subjected to IR exposure resembling typical assembly, and tested for in vitro drug release as described below. Corbion PC17 is a high molecular weight PCL while Corbion PC04 is a low molecular weight PCL.

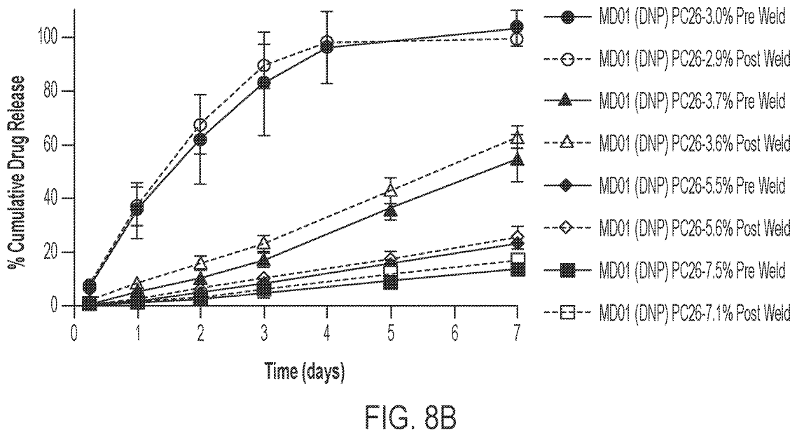

In Vitro Release: MD01 was evaluated for release in fasted state simulated gastric fluid (FaSSGF) for seven days. Drug arms within a general range of approximately 25-150 mg, typically weighing approximately 100 mg, were used to evaluate in vitro release. Drug arms were pan-coated with PC26, and evaluated for drug release kinetics before and after exposure to welding conditions (IR exposure to 4 to 7 mm out of the 14 mm drug arm) according to Example 4. The cumulative drug release was plotted and shown in FIG. 7.

As shown in FIG. 7, release of both memantine and donepezil could be modulated and controlled by use of an appropriate release-rate modulating film. FIG. 7 further shows that exposure of the coated arms to welding conditions did not affect the drug release rate over at least 7 days, indicating that the release modulation afforded by PC26 coating formulation would not be adversely affected by the welding process used in gastric residence system assembly.

### Example 10: Effect of Incremental Coating with PC26 on Drug Release Kinetics for Welded Gastric Residence System with Low Load Memantine/Donepezil Formulation (MD01)

To elucidate how incremental PC26 coating (75:25 w/w, Corbion PC17: Corbion PC04; +2% Mg stearate by weight of solids) affects memantine and donepezil drug release in residence systems, drug arms for MD01 were prepared, pan-coated with PC26 as described in Example 3 to achieve a coat weight gain of approximately 3%, 3.5%, 5.5% or 7% to 7.5%, subjected to IR exposure resembling that in typical assembly, and subsequently tested for in vitro drug release as described below.

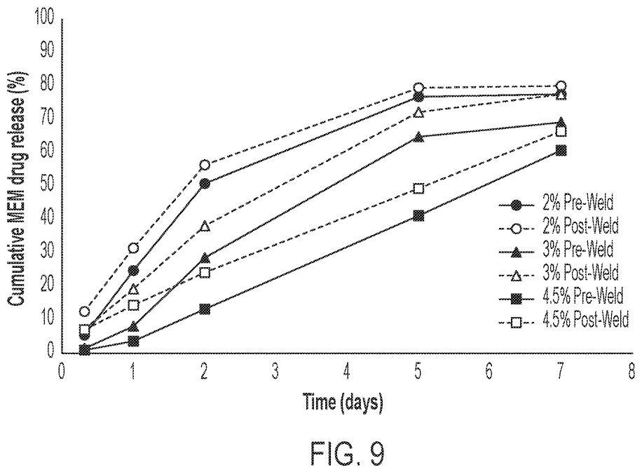

In Vitro Release: MD01 was evaluated for release in fasted state simulated gastric fluid (FaSSGF) for seven days. Drug arms within a general range of approximately 25-150 mg, typically weighing approximately 100 mg, were used to evaluate in vitro release. Drug arms were pan-coated with PC26, and evaluated for drug release kinetics before and after exposure to welding conditions (IR exposure to 4 to 7 mm out of the 14 mm drug arm) according to Example 4. The average coat wait gain for the respective groups of drug arms were as displayed in FIGS. 8A and 8B. The cumulative drug release with PC26 coating at the indicated coat weight gains was compared, and shown in FIG. 8A (memantine release) and FIG. 8B (donepezil release).

As shown in FIGS. 8A and 8B, release of both memantine and donepezil could be modulated and controlled by use of an appropriate release-rate modulating film at a selected coating mass. PC26 coating at 3% mass gain afforded linear release kinetics through day 4, at which point most of the drugs had been released. Coating at 3.5% mass gain afforded a more gradual release of both drugs. Coating at 5.5% and 7% mass gain resulted in linear release kinetics for both memantine and donepezil, but also resulted in relatively low cumulative drug release (FIGS. 8A and 8B, respectively). FIGS. 8A and 8B further showed that heat exposure of the coated arms did not substantially affect the drug release rates over at least 7 days, indicating that the release modulation afforded by PC26 coating formulation (at 3% to 7.5% coat weight gain) would not be adversely affected by the welding process used in gastric residence system assembly.

### Example 11: Effect of Incremental Coating with PC27 on Drug Release Kinetics for Welded Gastric Residence System with Low Load Memantine Formulation (MEM116)

To elucidate how incremental coating with PC27 formulation (40:60 w/w, Corbion PC17: Corbion PC02; +2% Mg stearate by weight of solids) affects memantine drug release in residence systems, drug arms for MEM116 was prepared, pan-coated with PC27 as described in Example 3 to achieve a coat weight gain of approximately 2%, 3%, or 4.5%, subjected to IR exposure resembling typical assembly, and subsequently tested for in vitro drug release as described below.

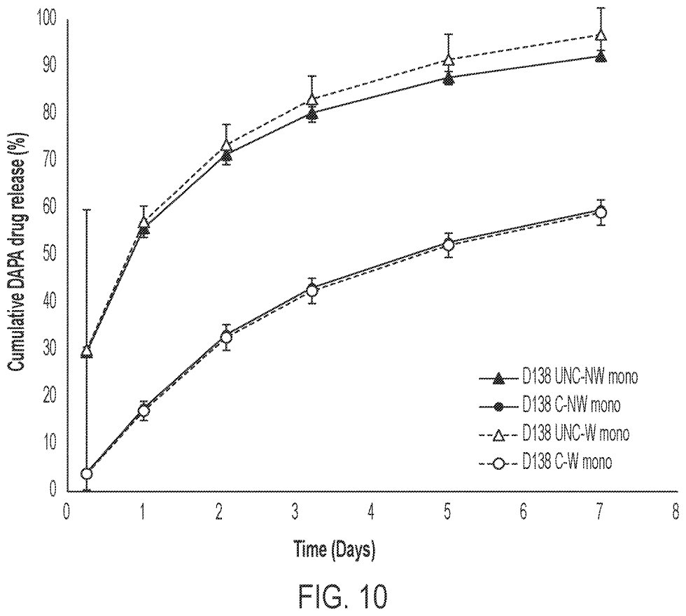

In Vitro Release: MEM116 was evaluated for release in fasted state simulated gastric fluid (FaSSGF) for seven days. Drug arms within a general range of approximately 25-150 mg, typically weighing approximately 100 mg, were used to evaluate in vitro release. Drug arms were pan-coated with PC27, and evaluated for drug release kinetics before and after exposure to welding conditions (IR exposure to 4 to 7 mm out of the 14 mm drug arm) according to Example 4. The average coat weight gains for the respective groups of drug arms were as displayed in FIG. 9. The cumulative drug release with PC27 coating at the indicated coat weight gains is shown in FIG. 9.

As shown in FIG. 9, release of memantine could be modulated and controlled by use of an appropriate release-rate modulating film at a selected coating mass. PC27 coating at 4.5% mass gain resulted in linear release kinetics for memantine.

### Example 12: Effect of PDL/PDLG5002A Coating on Drug Release Kinetics for Welded Gastric Residence System with Dapagliflozin Formulation (D138)

To elucidate the effect of a candidate PDL-based coating on dapagliflozin drug release in residence systems, drug formulation rods (monoliths) for D138 were prepared, pan-coated with PDL/PDLG5002A (1:1 w/w, PDL20: PDLG5002A; +2% Mg stearate by weight of solids) using procedures as described in Example 3, subjected to IR exposure resembling typical assembly, and tested for in vitro drug release as described below.

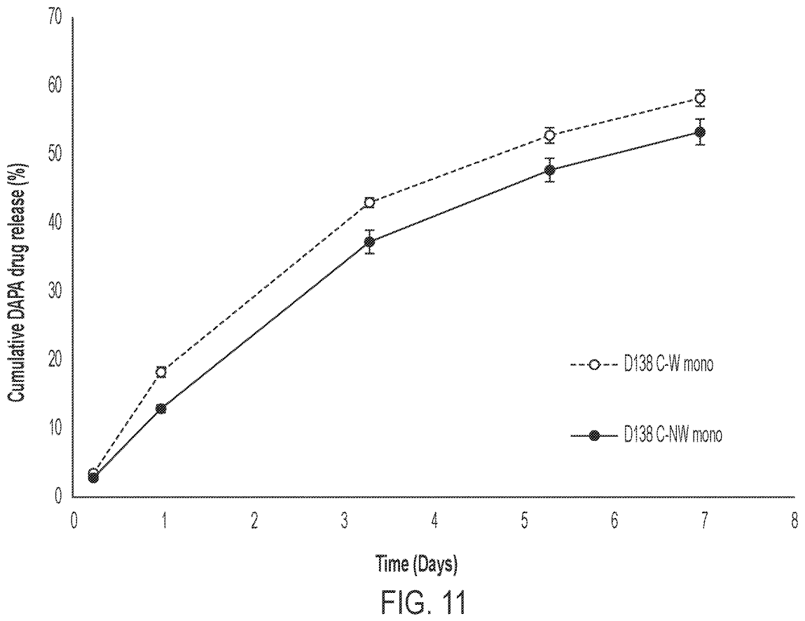

In Vitro Release: D138 was evaluated for release in fasted state simulated gastric fluid (FaSSGF) for seven days. Drug arms within a general range of approximately 25-150 mg, typically weighing approximately 100 mg, were used to evaluate in vitro release. Drug arms were prepared (i) with or without coating, and (ii) before and after exposure to welding conditions (IR exposure to 4 mm out of the 10 mm drug arm), and evaluated for drug release kinetics according to Example 4. The cumulative drug release was plotted and shown in FIG. 10 (UNC-NW mono=uncoated, non-welded monoliths; C-NW mono=coated, non-welded monoliths; UNC-W mono=uncoated, welded monoliths; C-W mono=coated, welded monoliths).

FIG. 10 further showed that exposure of the coated monoliths to welding conditions did not affect drug release rate over at least 7 days as compared to the coated monoliths not exposed to welding conditions, indicating that the release modulation afforded by the PDL/PDLG5002A coating formulation would not be adversely affected by the welding process used in gastric residence system assembly.

### Example 13: Effect of PDL/PDLG5002A Coating on Drug Release Kinetics for Welded Gastric Residence System with Dapagliflozin Formulation (D138) Receiving Over-Exposure to Welding

To determine whether PDL/PDLG5002A coating could withstand excessive exposure to heat and still retain a dapagliflozin drug release profile similar to the pre-exposure drug release profile, drug monoliths for D138 were prepared, pan-coated with PDL/PDLG5002A (1:1 w/w, PDL20: PDLG5002A; +2% Mg stearate by weight of solids) using procedures as described in Example 3, subjected to IR exposure exceeding that experienced in a typical assembly process, and tested for in vitro drug release as described below.

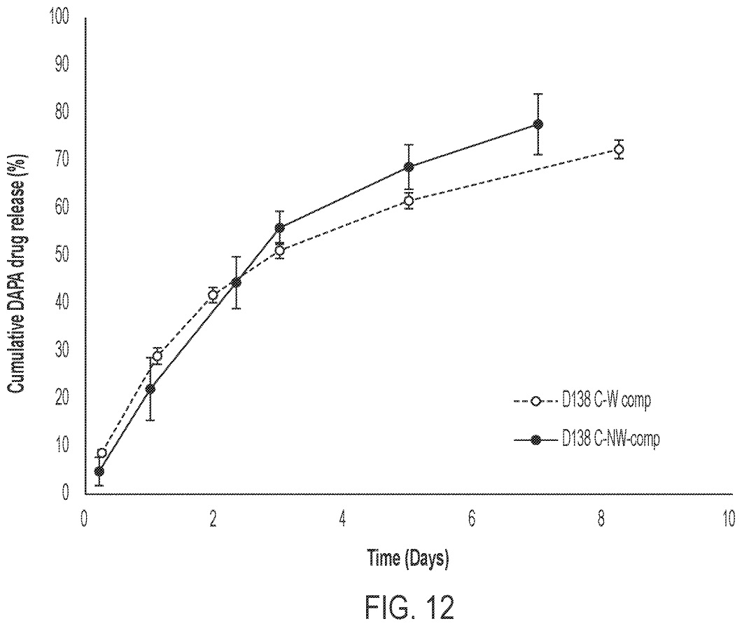

In Vitro Release: D138 was evaluated for release in fasted state simulated gastric fluid (FaSSGF) for seven days. Drug arms within a general range of approximately 25-150 mg, typically weighing approximately 100 mg, were used to evaluate in vitro release. Drug arms were prepared, pan-coated with PDL/PDLG5002A with or without IR exposure exceeding that in welding for typical assembly (IR exposure to 15 mm out of the 15 mm drug arm), and evaluated for drug release kinetics according to Example 4. The cumulative drug release was plotted and shown in FIG. 11.

FIG. 11 shows that exposure to welding conditions, with IR exposure exceeding that experienced during typical assembly of the coated monoliths, did not substantially affect the drug release rate over at least 7 days, indicating that the release modulation afforded by PDL/PDLG5002A coating formulation would not be adversely affected by the welding process in a typical gastric residence system assembly process, or an assembly procedure where even more exposure to IR irradiation occurs than the exposure that occurs during the typical assembly process.

### Example 14: Effect of PDL/PDLG5002A Coating on Drug Release Kinetics for Gastric Residence System with Dapagliflozin Formulation (D138) Receiving Overexposure to Welding

To determine whether PDL/PDLG5002A coating could withstand excessive exposure to welding in drug arm assembly and still retain a drug release profile similar to the pre-exposure drug release profile, composite drug arms containing D138 as well as inactive arm-parts were prepared, pan-coated with PDL/PDLG5002A (1:1 w/w, PDL20: PDLG5002A; +2% Mg stearate by weight of solids) using procedures as described in Example 3, subjected to IR exposure exceeding that in typical assembly and tested for in vitro drug release as described below.

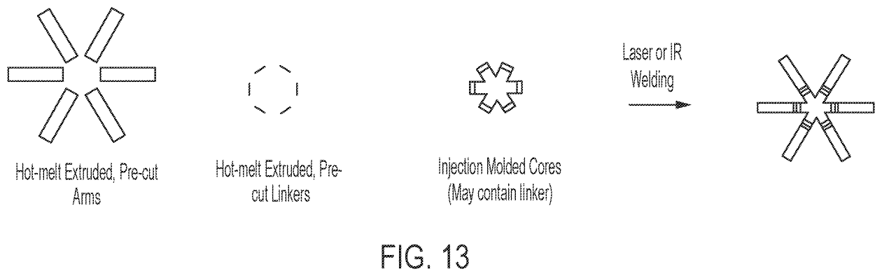

In Vitro Release: D138 was evaluated for release in fasted state simulated gastric fluid (FaSSGF) for seven days. Drug arms within a general range of approximately 25-150 mg, typically weighing approximately 100 mg, were used to evaluate in vitro release. D138-containing composite drug arms were prepared, pan-coated with PDL/PDLG5002A with or without IR exposure exceeding that in welding for typical assembly (IR exposure to 15 mm out of the 15 mm drug arm), and evaluated for drug release kinetics according to Example 4. The cumulative drug release was plotted and shown in FIG. 12 (C-W comp=coated, welded composite arm; C-NW comp=coated, non-welded composite arm).

FIG. 12 shows that exposure to welding conditions, with IR exposure exceeding that in typical assembly of the coated composite arms, did not significantly affect drug release rate, indicating that the release modulation afforded by PDL/PDLG5002A coating formulation would not be adversely affected by the welding process in a typical gastric residence system, or an assembly procedure where even more exposure to IR irradiation occurs than the exposure that occurs during the typical assembly process.

### Exemplary Embodiments

Embodiment 1. An arm for use in a gastric residence system, comprising:

a carrier polymer,
at least one agent or a pharmaceutically acceptable salt thereof, and
a release rate-modulating film coated on at least a portion of the surface of the arm;
wherein the release rate-modulating film comprises poly-D,L-lactide (PDL) and poly-D,L-lactide/glycolide (PDLG).

Embodiment 2. The arm of embodiment 1, wherein the PDL comprises PDL having an intrinsic viscosity of about 1 dl/g to about 4 dl/g.

Embodiment 3. The arm of embodiment 1, wherein the PDLG comprises PDLG having an intrinsic viscosity of about 0.1 dl/g to about 3 dl/g; 0.1 dl/g to about 1.5 dl/g; or 0.1 dl/g to about 0.5 dl/g.

Embodiment 4. The arm of any one of embodiments 1-3, wherein the PDL:PDLG ratio is between about 2:1 to about 1:2 (weight/weight).

Embodiment 5. The arm of any one of embodiments 1-3, wherein the PDL:PDLG ratio is between about 1.25:1 to about 1:1.25 (w/w).

Embodiment 6. The arm of any one of embodiments 1-3, wherein the PDL:PDLG ratio is about 1:1 (w/w).

Embodiment 7. The arm of any one of embodiments 1-6, wherein the release rate-modulating film is substantially free of porogen.

Embodiment 8. The arm of any one of embodiments 1-7, wherein the increase in the weight of the arm due to addition of the release rate-modulating film is about 2% to about 6% of the weight of the uncoated arm.

Embodiment 9. The arm of any one of embodiments 1-8, wherein the release rate of agent from the arm in aqueous media is substantially linear over at least a 96-hour period.

Embodiment 10. The arm of any one of embodiments 1-9, wherein the release rate of agent from the arm is substantially the same before and after thermal cycling.

Embodiment 11. A gastric residence system comprising an arm of any one of embodiments 1-10.

Embodiment 12. A gastric residence system comprising:

one or more arms of any one of embodiments 1-10; and

a central elastic polymeric component;

wherein the one or more arms are each connected to the central elastic polymeric component via a separate linker component;

wherein the gastric residence system is configured to be folded and physically constrained during administration and is configured to assume an open retention shape upon removal of a constraint;

wherein change between the folded shape and the open retention shape is mediated by the elastic polymeric component that undergoes elastic deformation when the residence system is in the folded shape and recoils when the gastric residence system assumes the open retention shape; and

wherein said linker degrades, dissolves, disassociates, or mechanically weakens in a gastric environment which results in loss of retention shape integrity and passage out of a gastric cavity.

Embodiment 13. An arm for use in a gastric residence system, comprising:

a carrier polymer,
at least one agent or a pharmaceutically acceptable salt thereof, and
a release rate-modulating film coated on at least a portion of the surface of the arm;
wherein the release rate-modulating film comprises high molecular weight polycaprolactone (PCL-HMW) and low molecular weight polycaprolactone (PCL-LMW).

Embodiment 14. The arm of embodiment 13, wherein the PCL-HMW comprises PCL of about Mₙ 75,000 to about Mₙ 250,000; or PCL having an intrinsic viscosity of about 1.0 dl/g to about 2.4 dl/g; or PCL having an intrinsic viscosity of about 1.2 dl/g to about 2.4 dl/g; or PCL having an intrinsic viscosity of about 1.6 dl/g to about 2.4 dl/g.

Embodiment 15. The arm of embodiment 13 or embodiment 14, wherein the PCL-LMW comprises PCL of about Mₙ 10,000 to about Mₙ 20,000; or PCL having an intrinsic viscosity of about 0.1 dl/g to about 0.8 dl/g.

Embodiment 16. The arm of embodiment 13, wherein the PCL-HMW comprises PCL of about Mₙ 75,000 to about Mₙ 250,000, or PCL having an intrinsic viscosity of about 1.0 dl/g to about 2.4 dl/g, or PCL having an intrinsic viscosity of about 1.2 dl/g to about 2.4 dl/g, or PCL having an intrinsic viscosity of about 1.6 dl/g to about 2.4 dl/g; and the PCL-LMW comprises PCL of about Mₙ 10,000 to about Mₙ 20,000, or PCL having an intrinsic viscosity of about 0.1 dl/g to about 0.8 dl/g.

Embodiment 17. The arm of any one of embodiments 13-16, wherein the (PCL-HMW):(PCL-LMW) ratio is between about 1:4 to about 95:5 (weight/weight).

Embodiment 18. The arm of any one of embodiments 13-16, wherein the (PCL-HMW):(PCL-LMW) ratio is between about 2:3 to about 95:5 (weight/weight).

Embodiment 19. The arm of any one of embodiments 13-16, wherein the (PCL-HMW):(PCL-LMW) ratio is between about 3:1 to about 95:5 (weight/weight).

Embodiment 20. The arm of any one of embodiments 13-16, wherein the (PCL-HMW):(PCL-LMW) ratio is about 9:1 (w/w).

Embodiment 21. The arm of any one of embodiments 13-16, wherein the (PCL-HMW):(PCL-LMW) ratio is about 1:3 (w/w).

Embodiment 22. The arm of any one of embodiments 13-16, wherein the (PCL-HMW):(PCL-LMW) ratio is about 4:6 (w/w); or wherein the (PCL-HMW):(PCL-LMW) ratio is about 6:4 (w/w).

Embodiment 23. The arm of any one of embodiments 13-16, wherein the (PCL-HMW):(PCL-LMW) ratio is about 1:1 (w/w).

Embodiment 24. The arm of any one of embodiments 13-16, wherein the (PCL-HMW):(PCL-LMW) ratio is about 3:1 (w/w).

Embodiment 25. The arm of any one of embodiments 13-16, wherein the (PCL-HMW):(PCL-LMW) ratio is about 85:15 (w/w).

Embodiment 26. The arm of any one of embodiments 13-16, wherein the release rate-modulating film is substantially free of porogen.

Embodiment 27. The arm of any one of embodiments 13-26, wherein the increase in the weight of the arm due to addition of the release rate-modulating film is about 2% to about 6% of the weight of the uncoated arm.

Embodiment 28. The arm of any one of embodiments 13-27, wherein the release rate of agent from the arm in aqueous media is substantially linear over at least a 96-hour period.

Embodiment 29. The arm of any one of embodiments 13-28, wherein the release rate of agent from the arm is substantially the same before and after thermal cycling.

Embodiment 30. A gastric residence system comprising an arm of any one of embodiments 13-29.

Embodiment 31. A gastric residence system comprising:

one or more arms of any one of embodiments 13-29; and

a central elastic polymeric component;

wherein the one or more arms are each connected to the central elastic polymeric component via a separate linker component;

wherein the gastric residence system is configured to be folded and physically constrained during administration and is configured to assume an open retention shape upon removal of a constraint;

wherein change between the folded shape and the open retention shape is mediated by the elastic polymeric component that undergoes elastic deformation when the residence system is in the folded shape and recoils when the gastric residence system assumes the open retention shape; and

wherein said linker degrades, dissolves, disassociates, or mechanically weakens in a gastric environment which results in loss of retention shape integrity and passage out of a gastric cavity.

Embodiment 32. An arm for use in a gastric residence system, comprising:

a carrier polymer,
at least one agent or a pharmaceutically acceptable salt thereof, and
a release rate-modulating film coated on at least a portion of the surface of the arm;
wherein the release rate-modulating film comprises poly-D,L-lactide (PDL).

Embodiment 33. The arm of embodiment 32, wherein the PDL comprises PDL having an intrinsic viscosity of about 1 dl/g to about 5 dl/g, or about 1.6 dl/g to about 2.4 dl/g.

Embodiment 34. The arm of embodiment 32 or embodiment 33, wherein the release rate-modulating film further comprises polycaprolactone (PCL).

Embodiment 35. The arm of embodiment 32 or embodiment 33, wherein the release rate-modulating film further comprises polycaprolactone (PCL) and polyethylene glycol (PEG).

Embodiment 36. The arm of embodiment 32 or embodiment 33, wherein the release rate-modulating film further comprises polycaprolactone (PCL), polyethylene glycol (PEG) and polypropylene glycol (PPG).

Embodiment 37. The arm of any one of embodiments 34-36, wherein the PCL comprises PCL of about Mₙ 75,000 to about Mₙ 250,000.

Embodiment 38. The arm of any one of embodiments 35-37, wherein the PEG comprises PEG of about Mₙ 800 to about Mₙ 20,000.

Embodiment 39. The arm of any one of embodiments 36-38, wherein the PPG comprises PPG having Mₙ of at least about 2,500.

Embodiment 40. The arm of any one of embodiments 36-38, wherein the PPG comprises PPG of about Mₙ 2,500 to about Mₙ 6,000.

Embodiment 41. The arm of any one of embodiments 34-39, wherein the PDL:PCL ratio is about 9:27 (w/w).

Embodiment 42. The arm of any one of embodiments 34-39, wherein the PDL:PCL ratio is about 36:9 (w/w).

Embodiment 43. The arm of any one of embodiments 36-39, wherein the PDL:PCL:PEG ratio is about 9:27:4 (w/w/w).

Embodiment 44. The arm of any one of embodiments 36-39, wherein the PDL:PCL:PEG ratio is about 36:9:5 (w/w/w).

Embodiment 45. The arm of any one of embodiments 32-44, wherein the release rate-modulating film is substantially free of porogen.

Embodiment 46. The arm of any one of embodiments 32-45, wherein the increase in the weight of the arm due to addition of the release rate-modulating film is about 2% to about 6% of the weight of the uncoated arm.

Embodiment 47. The arm of any one of embodiments 32-46, wherein the release rate of agent from the arm in aqueous media is substantially linear over at least a 96-hour period.

Embodiment 48. The arm of any one of embodiments 32-47, wherein the release rate of agent from the arm is substantially the same before and after thermal cycling.

Embodiment 49. A gastric residence system comprising an arm of any one of embodiments 32-48.

Embodiment 50. A gastric residence system comprising:

one or more arms of any one of embodiments 32-48; and

a central elastic polymeric component;

wherein the one or more arms are each connected to the central elastic polymeric component via a separate linker component;

wherein the gastric residence system is configured to be folded and physically constrained during administration and is configured to assume an open retention shape upon removal of a constraint;

wherein change between the folded shape and the open retention shape is mediated by the elastic polymeric component that undergoes elastic deformation when the residence system is in the folded shape and recoils when the gastric residence system assumes the open retention shape; and

wherein said linker degrades, dissolves, disassociates, or mechanically weakens in a gastric environment which results in loss of retention shape integrity and passage out of a gastric cavity.

Embodiment 51. An arm for use in a gastric residence system, comprising:

a carrier polymer,
at least one agent or a pharmaceutically acceptable salt thereof, and
a release rate-modulating film coated on at least a portion of the surface of the arm;
wherein the release rate-modulating film comprises polycaprolactone (PCL).

Embodiment 52. The arm of embodiment 51, wherein the PCL comprises PCL of about Mₙ 75,000 to about Mₙ 250,000.

Embodiment 53. The arm of embodiment 51 or embodiment 52, wherein the release rate-modulating film further comprises polyethylene glycol (PEG).

Embodiment 54. The arm of embodiment 51 or embodiment 52, wherein the release rate-modulating film further comprises polyethylene glycol (PEG) and polypropylene glycol (PPG).

Embodiment 55. The arm of any one of embodiments 53-54, wherein the PEG comprises PEG of Mₙ about 800 to about 1,200.

Embodiment 56. The arm of any one of embodiments 54-55, wherein the PPG comprises PPG of about Mₙ 2,500 to about Mₙ 6,000.

Embodiment 57. The arm of any one of embodiments 54-55, wherein the PCL comprises between about 15% to about 80% of the release rate-modulating film, the PEG comprises between about 5% to about 15% of the release rate-modulating film, and/or the PPG comprises between about 5% to about 15% of the release rate-modulating film by weight.

Embodiment 58. The arm of any one of embodiments 51-57, wherein the release rate-modulating film is substantially free of porogen.

Embodiment 59. The arm of any one of embodiments 51-58, wherein the increase in the weight of the arm due to addition of the release rate-modulating film is about 2% to about 6% of the weight of the uncoated arm.

Embodiment 60. The arm of any one of embodiments 51-59, wherein the release rate of agent from the arm in aqueous media is substantially linear over at least a 96-hour period.

Embodiment 61. The arm of any one of embodiments 51-60, wherein the release rate of agent from the arm is substantially the same before and after thermal cycling.

Embodiment 62. A gastric residence system comprising an arm of any one of embodiments 51-61.

Embodiment 63. A gastric residence system comprising:

one or more arms of any one of embodiments 51-61; and

a central elastic polymeric component;

wherein the one or more arms are each connected to the central elastic polymeric component via a separate linker component;

wherein the gastric residence system is configured to be folded and physically constrained during administration and is configured to assume an open retention shape upon removal of a constraint;

wherein change between the folded shape and the open retention shape is mediated by the elastic polymeric component that undergoes elastic deformation when the residence system is in the folded shape and recoils when the gastric residence system assumes the open retention shape; and

wherein said linker degrades, dissolves, disassociates, or mechanically weakens in a gastric environment which results in loss of retention shape integrity and passage out of a gastric cavity.

Embodiment 64. An arm for use in a gastric residence system, comprising:

a carrier polymer,
at least one agent or a pharmaceutically acceptable salt thereof, and
a release rate-modulating film coated on at least a portion of the surface of the arm;
wherein the release rate-modulating film comprises high molecular weight poly-D,L-lactide (PDL-HMW) and low molecular weight poly-D,L-lactide (PDL-LMW).

Embodiment 65. The arm of embodiment 64, wherein the PDL-HMW comprises PDL of inherent viscosity of about 1.6 dl/g to about 2.4 dl/g.

Embodiment 66. The arm of embodiment 64 or embodiment 65, wherein the PDL-LMW comprises PDL of inherent viscosity of about 0.5 dl/g to about 1.5 dl/g.

Embodiment 67. The arm of embodiment 64, wherein the PDL-HMW comprises PDL having an intrinsic viscosity midpoint of about 2 dl/g and the PDL-LMW comprises PDL having an intrinsic viscosity midpoint of about 1.5 dl/g.

Embodiment 68. The arm of any one of embodiments 64-67, wherein the (PDL-HMW):(PDL-LMW) ratio is between about 5:95 to about 95:5 (weight/weight).

Embodiment 69. The arm of any one of embodiments 64-67, wherein the (PDL-HMW):(PDL-LMW) ratio is between about 2:3 to about 95:5 (weight/weight).

Embodiment 70. The arm of any one of embodiments 54-67, wherein the (PDL-HMW):(PDL-LMW) ratio is between about 3:1 to about 95:5 (weight/weight).

Embodiment 71. The arm of any one of embodiments 64-67, wherein the (PDL-HMW):(PDL-LMW) ratio is about 9:1 (w/w).

Embodiment 72. The arm of embodiment 64 or embodiment 65, wherein the release rate-modulating film further comprises polycaprolactone (PCL) and polyethylene glycol (PEG).

Embodiment 73. The arm of embodiment 72, wherein the PCL comprises PCL of about Mₙ 80,000 to about Mₙ 200,000.

Embodiment 74. The arm of embodiment 72 or 73, wherein the PEG comprises PEG of about Mₙ 1000 to about Mₙ 20,000.

Embodiment 75. The arm of any one of embodiments 72-74, wherein the (PDL-HMW+PDL-LMW) comprises between about 15% to about 80% of the release rate-modulating film, the PCL comprises between about 15% to about 75% of the release rate-modulating film, and the PEG comprises between about 5% to about 15% of the release rate-modulating film, by weight.

Embodiment 76. The arm of any one of embodiments 72-74, wherein the (PDL-HMW+PDL-LMW):PCL:PEG ratio is about 9:27:4 (w/w/w).

Embodiment 77. The arm of any one of embodiments 72-74, wherein the (PDL-HMW+PDL-LMW):PCL:PEG ratio is about 36:9:5 (w/w/w).

Embodiment 78. The arm of any one of embodiments 64-77, wherein the release rate-modulating film is substantially free of porogen.

Embodiment 79. The arm of any one of embodiments 64-78, wherein the increase in the weight of the arm due to addition of the release rate-modulating film is about 2% to about 6% of the weight of the uncoated arm.

Embodiment 80. The arm of any one of embodiments 64-79, wherein the release rate of agent from the arm in aqueous media is substantially linear over at least a 96-hour period.

Embodiment 81. The arm of any one of embodiments 64-80, wherein the release rate of agent from the arm is substantially the same before and after thermal cycling.

Embodiment 82. A gastric residence system comprising an arm of any one of embodiments 64-81.

Embodiment 83. A gastric residence system comprising:

one or more arms of any one of embodiments 64-81; and

a central elastic polymeric component;

wherein the one or more arms are each connected to the central elastic polymeric component via a separate linker component;

wherein the gastric residence system is configured to be folded and physically constrained during administration and is configured to assume an open retention shape upon removal of a constraint;

wherein change between the folded shape and the open retention shape is mediated by the elastic polymeric component that undergoes elastic deformation when the residence system is in the folded shape and recoils when the gastric residence system assumes the open retention shape; and

wherein said linker degrades, dissolves, disassociates, or mechanically weakens in a gastric environment which results in loss of retention shape integrity and passage out of a gastric cavity.

Embodiment 84. The arm of any one of embodiments 32, 51, or 64, wherein the release rate-modulating film further comprises a polyethylene glycol-polypropylene glycol-polyethylene glycol (PEG-PPG-PEG) block copolymer.

Embodiment 85. The arm of embodiment 84, wherein the PEG-PPG-PEG block copolymer comprises PEG-PPG-PEG block copolymer of Mₙ about 14,000 to about 15,000.

Embodiment 86. The arm of embodiment 84 or embodiment 85, wherein the PEG-PPG-PEG block copolymer comprises about 75% to about 90% ethylene glycol.

Embodiment 87. The arm of any one of embodiments 84-86, wherein the release rate-modulating film comprises PDL and PEG-PPG-PEG block copolymer, and wherein the (PDL):(PEG-PPG-PEG block copolymer) ratio is between about 85:15 to about 95:5 (w/w).

Embodiment 88. The arm of any one of embodiments 84-86, wherein the release rate-modulating film comprises PDL-HMW+PDL-LMW and PEG-PPG-PEG block copolymer, wherein the (PDL-HMW+PDL-LMW):(PEG-PPG-PEG block copolymer) ratio is between about 85:15 to about 95:5 (w/w).

Embodiment 89. The arm of any one of embodiments 84-86, wherein the release rate-modulating film comprises PCL and PEG-PPG-PEG block copolymer, wherein the (PCL):(PEG-PPG-PEG block copolymer) ratio is between about 85:15 to about 95:5 (w/w).

Embodiment 90. The arm of any one of embodiments 84-86, wherein the release rate-modulating film comprises PDL and PEG-PPG-PEG block copolymer, and wherein the (PDL):(PEG-PPG-PEG block copolymer) ratio is about 9:1 (w/w).

Embodiment 91. The arm of any one of embodiments 84-86, wherein the release rate-modulating film comprises PDL-HMW+PDL-LMW and PEG-PPG-PEG block copolymer, wherein the (PDL-HMW+PDL-LMW):(PEG-PPG-PEG block copolymer) ratio is about 9:1 (w/w).

Embodiment 92. The arm of any one of embodiments 84-86, wherein the release rate-modulating film comprises PCL and PEG-PPG-PEG block copolymer, wherein the (PCL):(PEG-PPG-PEG block copolymer) ratio is about 9:1 (w/w).

Embodiment 93. The arm of any one of embodiments 84-92, wherein the release rate-modulating film is substantially free of porogen.

Embodiment 94. The arm of any one of embodiments 84-93, wherein the increase in the weight of the arm due to addition of the release rate-modulating film is about 2% to about 6% of the weight of the uncoated arm.

Embodiment 95. The arm of any one of embodiments 84-94, wherein the release rate of agent from the arm in aqueous media is substantially linear over at least a 96-hour period.

Embodiment 96. The arm of any one of embodiments 84-95, wherein the release rate of agent from the arm is substantially the same before and after thermal cycling.

Embodiment 97. A gastric residence system comprising an arm of any one of embodiments 84-96.

Embodiment 98. A gastric residence system comprising:

one or more arms of any one of embodiments 84-96; and

a central elastic polymeric component;

wherein the one or more arms are each connected to the central elastic polymeric component via a separate linker component;

wherein the gastric residence system is configured to be folded and physically constrained during administration and is configured to assume an open retention shape upon removal of a constraint;

wherein change between the folded shape and the open retention shape is mediated by the elastic polymeric component that undergoes elastic deformation when the residence system is in the folded shape and recoils when the gastric residence system assumes the open retention shape; and

wherein said linker degrades, dissolves, disassociates, or mechanically weakens in a gastric environment which results in loss of retention shape integrity and passage out of a gastric cavity.

Embodiment 99. The arm of embodiment 32, wherein the release rate-modulating film further comprises polyethylene glycol (PEG).

Embodiment 100. The arm of embodiment 32, wherein the release rate-modulating film further comprises polypropylene glycol (PPG).

Embodiment 101. The arm of any one of embodiments 32, 51, or 64, wherein the release rate-modulating film further comprises polyethylene glycol (PEG) and polypropylene glycol (PPG).

Embodiment 102. The arm of embodiment 101, wherein the PDL comprises between about 75% to about 95% of the release rate-modulating film, the PEG comprises between about 3% to about 10% of the release rate-modulating film, and the PPG comprises between about 1% to about 7% of the release rate-modulating film, by weight.

Embodiment 103. The arm of embodiment 101, wherein the release rate-modulating film comprises PDL, PEG, and PPG, and wherein the (PDL):(PEG):(PPG) ratio is about 90:(six and two-thirds):(three and one-third) by weight.

Embodiment 104. The arm of embodiment 101, wherein the release rate-modulating film comprises PDL, PEG, PPG, wherein the (PDL):(PEG):(PPG) ratio is about 27:2:1 by weight.

Embodiment 105. The arm of embodiment 101, wherein the release rate-modulating film comprises PCL, PEG, PPG, wherein the (PCL):(PEG):(PPG) ratio is about 27:2:1 by weight.

Embodiment 106. The arm of embodiment 101, wherein the release rate-modulating film comprises (PDL-HMW+PDL-LMW), PEG, PPG, wherein the (PDL-HMW+PDL-LMW):(PEG):(PPG) ratio is about 27:2:1 by weight.

Embodiment 107. The arm of any one of embodiments 99 or 101-106, wherein the PEG comprises PEG of Mₙ about 800 to about 1,200.

Embodiment 108. The arm of any one of embodiments 100-106, wherein the PPG comprises PPG of about Mₙ 2,500 to about Mₙ 6,000.

Embodiment 109. The arm of any one of embodiments 99-108, wherein the release rate-modulating film is substantially free of porogen.

Embodiment 110. The arm of any one of embodiments 99-109, wherein the increase in the weight of the arm due to addition of the release rate-modulating film is about 2% to about 6% of the weight of the uncoated arm.

Embodiment 111. The arm of any one of embodiments 99-110, wherein the release rate of agent from the arm in aqueous media is substantially linear over at least a 96-hour period.

Embodiment 112. The arm of any one of embodiments 99-111, wherein the release rate of agent from the arm is substantially the same before and after thermal cycling.

Embodiment 113. A gastric residence system comprising an arm of any one of embodiments 99-112.

Embodiment 114. A gastric residence system comprising:

one or more arms of any one of embodiments 99-112; and

a central elastic polymeric component;

wherein the one or more arms are each connected to the central elastic polymeric component via a separate linker component;

wherein the gastric residence system is configured to be folded and physically constrained during administration and is configured to assume an open retention shape upon removal of a constraint;

wherein change between the folded shape and the open retention shape is mediated by the elastic polymeric component that undergoes elastic deformation when the residence system is in the folded shape and recoils when the gastric residence system assumes the open retention shape; and

wherein said linker degrades, dissolves, disassociates, or mechanically weakens in a gastric environment which results in loss of retention shape integrity and passage out of a gastric cavity.

Embodiment 115. An arm for use in a gastric residence system, comprising:

a carrier polymer,
at least one agent or a pharmaceutically acceptable salt thereof, and
a release rate-modulating film coated on at least a portion of the surface of the arm;
wherein the release rate-modulating film comprises poly-D-lactide-polycaprolactone co-polymer (PDL-PCL copolymer).

Embodiment 116. The arm of embodiment 115, wherein PDL comprises between about 15% to about 90% of the PDL-PCL copolymer.

Embodiment 117. The arm of embodiment 115, wherein PDL comprises between about 15% to about 35% of the PDL-PCL copolymer.

Embodiment 118. The arm of embodiment 115, wherein PDL comprises between about 70% to about 90% of the PDL-PCL copolymer.

Embodiment 119. The arm of any one of embodiments 115-118, wherein the PDL-PCL copolymer comprises PDL-PCL copolymer having intrinsic viscosity of about 0.6 dl/g to about 4 dl/g, preferably about 0.6 dl/g to about 2 dl/g.

Embodiment 120. The arm of any one of embodiments 115-119, wherein the release rate-modulating film further comprises PEG.

Embodiment 121. The arm of embodiment 120, wherein the PEG comprises PEG of average molecular weight between about 800 and about 1,200.

Embodiment 122. The arm of embodiment 120 or embodiment 121, wherein the PDL-PCL copolymer comprises about 75% to about 95% of the release rate modulating film by weight and the PEG comprises about 5% to about 25% of the release rate modulating film by weight.

Embodiment 123. The arm of embodiment 120 or embodiment 121, wherein the PDL-PCL copolymer comprises about 90% of the release rate modulating film by weight and the PEG comprises about 10% of the release rate modulating film by weight.

Embodiment 124. The arm of embodiment 115, wherein:

(a) PDL comprises about 25% of the PDL-PCL copolymer; or
(b) PDL comprises about 80% of the PDL-PCL copolymer.

Embodiment 125. The arm of any one of embodiments 115-124, wherein the release rate-modulating film is substantially free of porogen.

Embodiment 126. The arm of any one of embodiments 115-125, wherein the increase in the weight of the arm due to addition of the release rate-modulating film is about 2% to about 6% of the weight of the uncoated arm.

Embodiment 127. The arm of any one of embodiments 115-126, wherein the release rate of agent from the arm in aqueous media is substantially linear over at least a 96-hour period.

Embodiment 128. The arm of any one of embodiments 115-127, wherein the release rate of agent from the arm is substantially the same before and after thermal cycling.

Embodiment 129. A gastric residence system comprising an arm of any one of embodiments 115-128.

Embodiment 130. A gastric residence system comprising:

one or more arms of any one of embodiments 115-129; and

a central elastic polymeric component;

wherein the one or more arms are each connected to the central elastic polymeric component via a separate linker component;

wherein the gastric residence system is configured to be folded and physically constrained during administration and is configured to assume an open retention shape upon removal of a constraint;

wherein change between the folded shape and the open retention shape is mediated by the elastic polymeric component that undergoes elastic deformation when the residence system is in the folded shape and recoils when the gastric residence system assumes the open retention shape; and

wherein said linker degrades, dissolves, disassociates, or mechanically weakens in a gastric environment which results in loss of retention shape integrity and passage out of a gastric cavity.

Embodiment 131. The arm of any one of embodiments 115-123, wherein the release rate-modulating film further comprises a polyethylene glycol-polypropylene glycol-polyethylene glycol (PEG-PPG-PEG) block copolymer.

Embodiment 132. The arm of embodiment 131, wherein the PEG-PPG-PEG block copolymer comprises PEG-PPG-PEG block copolymer of Mₙ about 14,000 to about 15,000.

Embodiment 133. The arm of embodiment 131 or embodiment 132, wherein the PEG-PPG-PEG block copolymer comprises about 75% to about 90% ethylene glycol.

Embodiment 134. The arm of any one of embodiments 131-133, wherein the (PDL-PCL copolymer):(PEG-PPG-PEG block copolymer) ratio is between about 85:15 to about 95:5 (w/w).

Embodiment 135. The arm of any one of embodiments 131-133, wherein the (PDL-PCL copolymer):(PEG-PPG-PEG block copolymer) ratio is about 9:1 (w/w).

Embodiment 136. The arm of embodiment 131-135, wherein:

(a) PDL comprises about 25% of the PDL-PCL copolymer; or
(b) PDL comprises about 80% to about 90% of the PDL-PCL copolymer.

Embodiment 137. The arm of any one of embodiments 131-136, wherein the release rate-modulating film is substantially free of porogen.

Embodiment 138. The arm of any one of embodiments 131-137, wherein the increase in the weight of the arm due to addition of the release rate-modulating film is about 2% to about 6% of the weight of the uncoated arm.

Embodiment 139. The arm of any one of embodiments 131-138, wherein the release rate of agent from the arm in aqueous media is substantially linear over at least a 96-hour period.

Embodiment 140. The arm of any one of embodiments 131-139, wherein the release rate of agent from the arm is substantially the same before and after thermal cycling.

Embodiment 141. A gastric residence system comprising an arm of any one of embodiments 131-140.

Embodiment 142. A gastric residence system comprising:

one or more arms of any one of embodiments 131-140; and

a central elastic polymeric component;

wherein the one or more arms are each connected to the central elastic polymeric component via a separate linker component;

wherein the gastric residence system is configured to be folded and physically constrained during administration and is configured to assume an open retention shape upon removal of a constraint;

wherein change between the folded shape and the open retention shape is mediated by the elastic polymeric component that undergoes elastic deformation when the residence system is in the folded shape and recoils when the gastric residence system assumes the open retention shape; and

wherein said linker degrades, dissolves, disassociates, or mechanically weakens in a gastric environment which results in loss of retention shape integrity and passage out of a gastric cavity.

Embodiment 143. The arm or gastric residence system of any one of embodiments 1-142, wherein the release rate-modulating film is applied by pan coating.

Embodiment 144. The arm or gastric residence system of any one of embodiments 1-142, wherein the release rate-modulating film is applied by dip coating.

Embodiment 145. The arm or gastric residence system of any one of embodiments 1-144, wherein the at least one agent or a pharmaceutically acceptable salt thereof comprises one or more of drug, a pro-drug, a biologic, a statin, rosuvastatin, a nonsteroidal anti-inflammatory drug (NSAID), meloxicam, a selective serotonin reuptake inhibitor (SSRs), escitalopram, citalopram, a blood thinner, clopidogrel, a steroid, prednisone, an antipsychotic, aripiprazole, risperidone, an analgesic, buprenorphine, an opioid antagonist, naloxone, an anti-asthmatic, montelukast, an anti-dementia drug, memantine, a cardiac glycoside, digoxin, an alpha blocker, tamsulosin, a cholesterol absorption inhibitor, ezetimibe, an anti-gout treatment, colchicine, an antihistamine, loratadine, cetirizine, an opioid, loperamide, a proton-pump inhibitor, omeprazole, an antiviral agent, entecavir, an antibiotic, doxycycline, ciprofloxacin, azithromycin, an anti-malarial agent, levothyroxine, a substance abuse treatment, methadone, varenicline, a contraceptive, a stimulant, caffeine, a nutrient, folic acid, calcium, iodine, iron, zinc, thiamine, niacin, vitamin C, vitamin D, biotin, a plant extract, a phytohormone, a vitamin, a mineral, a protein, a polypeptide, a polynucleotide, a hormone, an anti-inflammatory drug, an antipyretic, an antidepressant, an antiepileptic, an antipsychotic agent, a neuroprotective agent, an anti-proliferative, an anti-cancer agent, an antimigraine drug, a prostaglandin, an antimicrobial, an antifungals, an antiparasitic, an anti-muscarinic, an anxiolytic, a bacteriostatic, an immunosuppressant agent, a sedative, a hypnotic, a bronchodilator, a cardiovascular drug, an anesthetic, an anti-coagulant, an enzyme inhibitor, a corticosteroid, a dopaminergic, an electrolyte, a gastro-intestinal drug, a muscle relaxant, a parasympathomimetic, an anorectic, an anti-narcoleptics, quinine, lumefantrine, chloroquine, amodiaquine, pyrimethamine, proguanil, chlorproguanil-dapsone, a sulfonamide, sulfadoxine, sulfamethoxypyridazine, mefloquine, atovaquone, primaquine, halofantrine, doxycycline, clindamycin, artemisinin, an artemisinin derivative, artemether, dihydroartemisinin, arteether, or artesunate.

Embodiment 146. The arm or gastric residence system of any one of embodiments 1-144, wherein the at least one agent or a pharmaceutically acceptable salt thereof comprises memantine.

Embodiment 147. The arm or gastric residence system of any one of embodiments 1-144, wherein the at least one agent or a pharmaceutically acceptable salt thereof comprises donepezil.

Embodiment 148. The arm or gastric residence system of any one of embodiments 1-144, wherein the at least one agent or a pharmaceutically acceptable salt thereof comprises memantine and donepezil.

Embodiment 149. The arm or gastric residence system of any one of embodiments 1-144, wherein the at least one agent or a pharmaceutically acceptable salt thereof comprises risperidone.

Embodiment 150. The arm or gastric residence system of any one of embodiments 1-144, wherein the at least one agent or a pharmaceutically acceptable salt thereof comprises dapagliflozin.

The disclosures of all publications, patents, patent applications and published patent applications referred to herein by an identifying citation are hereby incorporated herein by reference in their entirety. Web sites references using “World-Wide-Web” at the beginning of the Uniform Resource Locator (URL) can be accessed by replacing “World-Wide-Web” with www.

Although the foregoing invention has been described in some detail by way of illustration and example for purposes of clarity of understanding, it is apparent to those skilled in the art that certain changes and modifications will be practiced. Therefore, the description and examples should not be construed as limiting the scope of the invention.

What is claimed is:

1. An arm for use in a gastric residence system, comprising:
a carrier polymer,
at least one agent or a pharmaceutically acceptable salt thereof, and
a release rate-modulating film coated on at least a portion of the surface of the arm;
wherein the release rate-modulating film comprises poly-D,L-lactide (PDL) and poly-D,L-lactide/glycolide (PDLG).

2. The arm of claim 1, wherein the PDL comprises PDL having an intrinsic viscosity of about 1 dl/g to about 4 dl/g.

3. The arm of claim 1, wherein the PDLG comprises PDLG having an intrinsic viscosity of about 0.1 dl/g to about 3 dl/g; 0.1 dl/g to about 1.5 dl/g; or 0.1 dl/g to about 0.5 dl/g.

4. The arm of any one of claims 1-3, wherein the PDL:PDLG ratio is between about 2:1 to about 1:2 (weight/weight).

5. The arm of any one of claims 1-3, wherein the PDL:PDLG ratio is between about 1.25:1 to about 1:1.25 (w/w).

6. The arm of any one of claims 1-3, wherein the PDL:PDLG ratio is about 1:1 (w/w).

7. The arm of any one of claims 1-6, wherein the release rate-modulating film is substantially free of porogen.

8. The arm of any one of claims 1-7, wherein the increase in the weight of the arm due to addition of the release rate-modulating film is about 2% to about 6% of the weight of the uncoated arm.

9. The arm of any one of claims 1-8, wherein the release rate of agent from the arm in aqueous media is substantially linear over at least a 96-hour period.

10. The arm of any one of claims 1-9, wherein the release rate of agent from the arm is substantially the same before and after thermal cycling.

11. A gastric residence system comprising an arm of any one of claims 1-10.

12. A gastric residence system comprising:
one or more arms of any one of claims 1-10; and
a central elastic polymeric component;
wherein the one or more arms are each connected to the central elastic polymeric component via a separate linker component;
wherein the gastric residence system is configured to be folded and physically constrained during administration and is configured to assume an open retention shape upon removal of a constraint;
wherein change between the folded shape and the open retention shape is mediated by the elastic polymeric component that undergoes elastic deformation when the residence system is in the folded shape and recoils when the gastric residence system assumes the open retention shape; and
wherein said linker degrades, dissolves, disassociates, or mechanically weakens in a gastric environment which results in loss of retention shape integrity and passage out of a gastric cavity.

13. An arm for use in a gastric residence system, comprising:
a carrier polymer,
at least one agent or a pharmaceutically acceptable salt thereof, and
a release rate-modulating film coated on at least a portion of the surface of the arm;
wherein the release rate-modulating film comprises high molecular weight polycaprolactone (PCL-HMW) and low molecular weight polycaprolactone (PCL-LMW).

14. The arm of claim 13, wherein the PCL-HMW comprises PCL of about Mₙ 75,000 to about Mₙ 250,000; or PCL having an intrinsic viscosity of about 1.0 dl/g to about 2.4 dl/g; or PCL having an intrinsic viscosity of about 1.2 dl/g to about 2.4 dl/g; or PCL having an intrinsic viscosity of about 1.6 dl/g to about 2.4 dl/g.

15. The arm of claim 13 or claim 14, wherein the PCL-LMW comprises PCL of about Mₙ 10,000 to about Mₙ 20,000; or PCL having an intrinsic viscosity of about 0.1 dl/g to about 0.8 dl/g.

16. The arm of claim 13, wherein the PCL-HMW comprises PCL of about Mₙ 75,000 to about Mₙ 250,000, or PCL having an intrinsic viscosity of about 1.0 dl/g to about 2.4 dl/g, or PCL having an intrinsic viscosity of about 1.2 dl/g to about 2.4 dl/g, or PCL having an intrinsic viscosity of about 1.6 dl/g to about 2.4 dl/g; and the PCL-LMW comprises PCL of about Mₙ 10,000 to about Mₙ 20,000, or PCL having an intrinsic viscosity of about 0.1 dl/g to about 0.8 dl/g.

17. The arm of any one of claims 13-16, wherein the (PCL-HMW):(PCL-LMW) ratio is between about 1:4 to about 95:5 (weight/weight).

18. The arm of any one of claims 13-16, wherein the (PCL-HMW):(PCL-LMW) ratio is between about 2:3 to about 95:5 (weight/weight).

19. The arm of any one of claims 13-16, wherein the (PCL-HMW):(PCL-LMW) ratio is between about 3:1 to about 95:5 (weight/weight).

20. The arm of any one of claims 13-16, wherein the (PCL-HMW):(PCL-LMW) ratio is about 9:1 (w/w).

21. The arm of any one of claims 13-16, wherein the (PCL-HMW):(PCL-LMW) ratio is about 1:3 (w/w).

22. The arm of any one of claims 13-16, wherein the (PCL-HMW):(PCL-LMW) ratio is about 4:6 (w/w); or wherein the (PCL-HMW):(PCL-LMW) ratio is about 6:4 (w/w).

23. The arm of any one of claims 13-16, wherein the (PCL-HMW):(PCL-LMW) ratio is about 1:1 (w/w).

24. The arm of any one of claims 13-16, wherein the (PCL-HMW):(PCL-LMW) ratio is about 3:1 (w/w).

25. The arm of any one of claims 13-16, wherein the (PCL-HMW):(PCL-LMW) ratio is about 85:15 (w/w).

26. The arm of any one of claims 13-16, wherein the release rate-modulating film is substantially free of porogen.

27. The arm of any one of claims 13-26, wherein the increase in the weight of the arm due to addition of the release rate-modulating film is about 2% to about 6% of the weight of the uncoated arm.

28. The arm of any one of claims 13-27, wherein the release rate of agent from the arm in aqueous media is substantially linear over at least a 96-hour period.

29. The arm of any one of claims 13-28, wherein the release rate of agent from the arm is substantially the same before and after thermal cycling.

30. A gastric residence system comprising an arm of any one of claims 13-29.

31. A gastric residence system comprising:
one or more arms of any one of claims 13-29; and
a central elastic polymeric component;
wherein the one or more arms are each connected to the central elastic polymeric component via a separate linker component;
wherein the gastric residence system is configured to be folded and physically constrained during administration and is configured to assume an open retention shape upon removal of a constraint;
wherein change between the folded shape and the open retention shape is mediated by the elastic polymeric component that undergoes elastic deformation when the residence system is in the folded shape and recoils when the gastric residence system assumes the open retention shape; and
wherein said linker degrades, dissolves, disassociates, or mechanically weakens in a gastric environment which results in loss of retention shape integrity and passage out of a gastric cavity.

32. An arm for use in a gastric residence system, comprising:
a carrier polymer,
at least one agent or a pharmaceutically acceptable salt thereof, and
a release rate-modulating film coated on at least a portion of the surface of the arm;
wherein the release rate-modulating film comprises poly-D,L-lactide (PDL).

33. The arm of claim 32, wherein the PDL comprises PDL having an intrinsic viscosity of about 1 dl/g to about 5 dl/g, or about 1.6 dl/g to about 2.4 dl/g.

34. The arm of claim 32 or claim 33, wherein the release rate-modulating film further comprises polycaprolactone (PCL).

35. The arm of claim 32 or claim 33, wherein the release rate-modulating film further comprises polycaprolactone (PCL) and polyethylene glycol (PEG).

36. The arm of claim 32 or claim 33, wherein the release rate-modulating film further comprises polycaprolactone (PCL), polyethylene glycol (PEG) and polypropylene glycol (PPG).

37. The arm of any one of claims 34-36, wherein the PCL comprises PCL of about Mₙ 75,000 to about Mₙ 250,000.

38. The arm of any one of claims 35-37, wherein the PEG comprises PEG of about Mₙ 800 to about Mₙ 20,000.

39. The arm of any one of claims 36-38, wherein the PPG comprises PPG having Mₙ of at least about 2,500.

40. The arm of any one of claims 36-38, wherein the PPG comprises PPG of about Mₙ 2,500 to about Mₙ 6,000.

41. The arm of any one of claims 34-39, wherein the PDL:PCL ratio is about 9:27 (w/w).

42. The arm of any one of claims 34-39, wherein the PDL:PCL ratio is about 36:9 (w/w).

43. The arm of any one of claims 36-39, wherein the PDL:PCL:PEG ratio is about 9:27:4 (w/w/w).

44. The arm of any one of claims 36-39, wherein the PDL:PCL:PEG ratio is about 36:9:5 (w/w/w).

45. The arm of any one of claims 32-44, wherein the release rate-modulating film is substantially free of porogen.

46. The arm of any one of claims 32-45, wherein the increase in the weight of the arm due to addition of the release rate-modulating film is about 2% to about 6% of the weight of the uncoated arm.

47. The arm of any one of claims 32-46, wherein the release rate of agent from the arm in aqueous media is substantially linear over at least a 96-hour period.

48. The arm of any one of claims 32-47, wherein the release rate of agent from the arm is substantially the same before and after thermal cycling.

49. A gastric residence system comprising an arm of any one of claims 32-48.

50. A gastric residence system comprising:
one or more arms of any one of claims 32-48; and
a central elastic polymeric component;
wherein the one or more arms are each connected to the central elastic polymeric component via a separate linker component;
wherein the gastric residence system is configured to be folded and physically constrained during administration and is configured to assume an open retention shape upon removal of a constraint;
wherein change between the folded shape and the open retention shape is mediated by the elastic polymeric component that undergoes elastic deformation when the residence system is in the folded shape and recoils when the gastric residence system assumes the open retention shape; and
wherein said linker degrades, dissolves, disassociates, or mechanically weakens in a gastric environment which results in loss of retention shape integrity and passage out of a gastric cavity.

51. An arm for use in a gastric residence system, comprising:
a carrier polymer,
at least one agent or a pharmaceutically acceptable salt thereof, and
a release rate-modulating film coated on at least a portion of the surface of the arm;
wherein the release rate-modulating film comprises polycaprolactone (PCL).

52. The arm of claim 51, wherein the PCL comprises PCL of about Mₙ 75,000 to about Mₙ 250,000.

53. The arm of claim 51 or claim 52, wherein the release rate-modulating film further comprises polyethylene glycol (PEG).

54. The arm of claim 51 or claim 52, wherein the release rate-modulating film further comprises polyethylene glycol (PEG) and polypropylene glycol (PPG).

55. The arm of any one of claims 53-54, wherein the PEG comprises PEG of Mₙ about 800 to about 1,200.

56. The arm of any one of claims 54-55, wherein the PPG comprises PPG of about Mₙ 2,500 to about Mₙ 6,000.

57. The arm of any one of claims 54-55, wherein the PCL comprises between about 15% to about 80% of the release rate-modulating film, the PEG comprises between about 5% to about 15% of the release rate-modulating film, and/or the PPG comprises between about 5% to about 15% of the release rate-modulating film by weight.

58. The arm of any one of claims 51-57, wherein the release rate-modulating film is substantially free of porogen.

59. The arm of any one of claims 51-58, wherein the increase in the weight of the arm due to addition of the release rate-modulating film is about 2% to about 6% of the weight of the uncoated arm.

60. The arm of any one of claims 51-59, wherein the release rate of agent from the arm in aqueous media is substantially linear over at least a 96-hour period.

61. The arm of any one of claims 51-60, wherein the release rate of agent from the arm is substantially the same before and after thermal cycling.

62. A gastric residence system comprising an arm of any one of claims 51-61.

63. A gastric residence system comprising:
one or more arms of any one of claims 51-61; and
a central elastic polymeric component;
wherein the one or more arms are each connected to the central elastic polymeric component via a separate linker component;
wherein the gastric residence system is configured to be folded and physically constrained during administration and is configured to assume an open retention shape upon removal of a constraint;
wherein change between the folded shape and the open retention shape is mediated by the elastic polymeric component that undergoes elastic deformation when the residence system is in the folded shape and recoils when the gastric residence system assumes the open retention shape; and
wherein said linker degrades, dissolves, disassociates, or mechanically weakens in a gastric environment which results in loss of retention shape integrity and passage out of a gastric cavity.

64. An arm for use in a gastric residence system, comprising:
a carrier polymer,
at least one agent or a pharmaceutically acceptable salt thereof, and
a release rate-modulating film coated on at least a portion of the surface of the arm;
wherein the release rate-modulating film comprises high molecular weight poly-D,L-lactide (PDL-HMW) and low molecular weight poly-D,L-lactide (PDL-LMW).

65. The arm of claim 64, wherein the PDL-HMW comprises PDL of inherent viscosity of about 1.6 dl/g to about 2.4 dl/g.

66. The arm of claim 64 or claim 65, wherein the PDL-LMW comprises PDL of inherent viscosity of about 0.5 dl/g to about 1.5 dl/g.

67. The arm of claim 64, wherein the PDL-HMW comprises PDL having an intrinsic viscosity midpoint of about 2 dl/g and the PDL-LMW comprises PDL having an intrinsic viscosity midpoint of about 1.5 dl/g.

68. The arm of any one of claims 64-67, wherein the (PDL-HMW):(PDL-LMW) ratio is between about 5:95 to about 95:5 (weight/weight).

69. The arm of any one of claims 64-67, wherein the (PDL-HMW):(PDL-LMW) ratio is between about 2:3 to about 95:5 (weight/weight).

70. The arm of any one of claims 54-67, wherein the (PDL-HMW):(PDL-LMW) ratio is between about 3:1 to about 95:5 (weight/weight).

71. The arm of any one of claims 64-67, wherein the (PDL-HMW):(PDL-LMW) ratio is about 9:1 (w/w).

72. The arm of claim 64 or claim 65, wherein the release rate-modulating film further comprises polycaprolactone (PCL) and polyethylene glycol (PEG).

73. The arm of claim 72, wherein the PCL comprises PCL of about Mₙ 80,000 to about Mₙ 200,000.

74. The arm of claim 72 or 73, wherein the PEG comprises PEG of about Mₙ 1000 to about Mₙ 20,000.

75. The arm of any one of claims 72-74, wherein the (PDL-HMW+PDL-LMW) comprises between about 15% to about 80% of the release rate-modulating film, the PCL comprises between about 15% to about 75% of the release rate-modulating film, and the PEG comprises between about 5% to about 15% of the release rate-modulating film, by weight.

76. The arm of any one of claims 72-74, wherein the (PDL-HMW+PDL-LMW):PCL:PEG ratio is about 9:27:4 (w/w/w).

77. The arm of any one of claims 72-74, wherein the (PDL-HMW+PDL-LMW):PCL:PEG ratio is about 36:9:5 (w/w/w).

78. The arm of any one of claims 64-77, wherein the release rate-modulating film is substantially free of porogen.

79. The arm of any one of claims 64-78, wherein the increase in the weight of the arm due to addition of the release rate-modulating film is about 2% to about 6% of the weight of the uncoated arm.

80. The arm of any one of claims 64-79, wherein the release rate of agent from the arm in aqueous media is substantially linear over at least a 96-hour period.

81. The arm of any one of claims 64-80, wherein the release rate of agent from the arm is substantially the same before and after thermal cycling.

82. A gastric residence system comprising an arm of any one of claims 64-81.

83. A gastric residence system comprising:
one or more arms of any one of claims 64-81; and
a central elastic polymeric component;
wherein the one or more arms are each connected to the central elastic polymeric component via a separate linker component;
wherein the gastric residence system is configured to be folded and physically constrained during administration and is configured to assume an open retention shape upon removal of a constraint;
wherein change between the folded shape and the open retention shape is mediated by the elastic polymeric component that undergoes elastic deformation when the residence system is in the folded shape and recoils when the gastric residence system assumes the open retention shape; and
wherein said linker degrades, dissolves, disassociates, or mechanically weakens in a gastric environment which results in loss of retention shape integrity and passage out of a gastric cavity.

84. The arm of any one of claims 32, 51, or 64, wherein the release rate-modulating film further comprises a polyethylene glycol-polypropylene glycol-polyethylene glycol (PEG-PPG-PEG) block copolymer.

85. The arm of claim 84, wherein the PEG-PPG-PEG block copolymer comprises PEG-PPG-PEG block copolymer of Mₙ about 14,000 to about 15,000.

86. The arm of claim 84 or claim 85, wherein the PEG-PPG-PEG block copolymer comprises about 75% to about 90% ethylene glycol.

87. The arm of any one of claims 84-86, wherein the release rate-modulating film comprises PDL and PEG-PPG-PEG block copolymer, and wherein the (PDL):(PEG-PPG-PEG block copolymer) ratio is between about 85:15 to about 95:5 (w/w).

88. The arm of any one of claims 84-86, wherein the release rate-modulating film comprises PDL-HMW+PDL-LMW and PEG-PPG-PEG block copolymer, wherein the (PDL-HMW+PDL-LMW):(PEG-PPG-PEG block copolymer) ratio is between about 85:15 to about 95:5 (w/w).

89. The arm of any one of claims 84-86, wherein the release rate-modulating film comprises PCL and PEG-PPG-PEG block copolymer, wherein the (PCL):(PEG-PPG-PEG block copolymer) ratio is between about 85:15 to about 95:5 (w/w).

90. The arm of any one of claims 84-86, wherein the release rate-modulating film comprises PDL and PEG-PPG-PEG block copolymer, and wherein the (PDL):(PEG-PPG-PEG block copolymer) ratio is about 9:1 (w/w).

91. The arm of any one of claims 84-86, wherein the release rate-modulating film comprises PDL-HMW+PDL-LMW and PEG-PPG-PEG block copolymer, wherein the (PDL-HMW+PDL-LMW):(PEG-PPG-PEG block copolymer) ratio is about 9:1 (w/w).

92. The arm of any one of claims 84-86, wherein the release rate-modulating film comprises PCL and PEG-PPG-PEG block copolymer, wherein the (PCL):(PEG-PPG-PEG block copolymer) ratio is about 9:1 (w/w).

93. The arm of any one of claims 84-92, wherein the release rate-modulating film is substantially free of porogen.

94. The arm of any one of claims 84-93, wherein the increase in the weight of the arm due to addition of the release rate-modulating film is about 2% to about 6% of the weight of the uncoated arm.

95. The arm of any one of claims 84-94, wherein the release rate of agent from the arm in aqueous media is substantially linear over at least a 96-hour period.

96. The arm of any one of claims 84-95, wherein the release rate of agent from the arm is substantially the same before and after thermal cycling.

97. A gastric residence system comprising an arm of any one of claims 84-96.

98. A gastric residence system comprising:
one or more arms of any one of claims 84-96; and
a central elastic polymeric component;
wherein the one or more arms are each connected to the central elastic polymeric component via a separate linker component;
wherein the gastric residence system is configured to be folded and physically constrained during administration and is configured to assume an open retention shape upon removal of a constraint;
wherein change between the folded shape and the open retention shape is mediated by the elastic polymeric component that undergoes elastic deformation when the residence system is in the folded shape and recoils when the gastric residence system assumes the open retention shape; and
wherein said linker degrades, dissolves, disassociates, or mechanically weakens in a gastric environment which results in loss of retention shape integrity and passage out of a gastric cavity.

99. The arm of claim 32, wherein the release rate-modulating film further comprises polyethylene glycol (PEG).

100. The arm of claim 32, wherein the release rate-modulating film further comprises polypropylene glycol (PPG).

101. The arm of any one of claims 32, 51, or 64, wherein the release rate-modulating film further comprises polyethylene glycol (PEG) and polypropylene glycol (PPG).

102. The arm of claim 101, wherein the PDL comprises between about 75% to about 95% of the release rate-modulating film, the PEG comprises between about 3% to about 10% of the release rate-modulating film, and the PPG comprises between about 1% to about 7% of the release rate-modulating film, by weight.

103. The arm of claim 101, wherein the release rate-modulating film comprises PDL, PEG, and PPG, and wherein the (PDL):(PEG):(PPG) ratio is about 90:(six and two-thirds):(three and one-third) by weight.

104. The arm of claim 101, wherein the release rate-modulating film comprises PDL, PEG, PPG, wherein the (PDL):(PEG):(PPG) ratio is about 27:2:1 by weight.

105. The arm of claim 101, wherein the release rate-modulating film comprises PCL, PEG, PPG, wherein the (PCL):(PEG):(PPG) ratio is about 27:2:1 by weight.

106. The arm of claim 101, wherein the release rate-modulating film comprises (PDL-HMW+PDL-LMW), PEG, PPG, wherein the (PDL-HMW+PDL-LMW):(PEG):(PPG) ratio is about 27:2:1 by weight.

107. The arm of any one of claims 99 or 101-106, wherein the PEG comprises PEG of Mₙ about 800 to about 1,200.

108. The arm of any one of claims 100-106, wherein the PPG comprises PPG of about Mₙ 2,500 to about Mₙ 6,000.

109. The arm of any one of claims 99-108, wherein the release rate-modulating film is substantially free of porogen.

110. The arm of any one of claims 99-109, wherein the increase in the weight of the arm due to addition of the release rate-modulating film is about 2% to about 6% of the weight of the uncoated arm.

111. The arm of any one of claims 99-110, wherein the release rate of agent from the arm in aqueous media is substantially linear over at least a 96-hour period.

112. The arm of any one of claims 99-111, wherein the release rate of agent from the arm is substantially the same before and after thermal cycling.

113. A gastric residence system comprising an arm of any one of claims 99-112.

114. A gastric residence system comprising:
one or more arms of any one of claims 99-112; and
a central elastic polymeric component;
wherein the one or more arms are each connected to the central elastic polymeric component via a separate linker component;
wherein the gastric residence system is configured to be folded and physically constrained during administration and is configured to assume an open retention shape upon removal of a constraint;
wherein change between the folded shape and the open retention shape is mediated by the elastic polymeric component that undergoes elastic deformation when the residence system is in the folded shape and recoils when the gastric residence system assumes the open retention shape; and
wherein said linker degrades, dissolves, disassociates, or mechanically weakens in a gastric environment which results in loss of retention shape integrity and passage out of a gastric cavity.

115. An arm for use in a gastric residence system, comprising:
a carrier polymer,
at least one agent or a pharmaceutically acceptable salt thereof, and
a release rate-modulating film coated on at least a portion of the surface of the arm;
wherein the release rate-modulating film comprises poly-D-lactide-polycaprolactone co-polymer (PDL-PCL copolymer).

116. The arm of claim 115, wherein PDL comprises between about 15% to about 90% of the PDL-PCL copolymer.

117. The arm of claim 115, wherein PDL comprises between about 15% to about 35% of the PDL-PCL copolymer.

118. The arm of claim 115, wherein PDL comprises between about 70% to about 90% of the PDL-PCL copolymer.

119. The arm of any one of claims 115-118, wherein the PDL-PCL copolymer comprises PDL-PCL copolymer having intrinsic viscosity of about 0.6 dl/g to about 4 dl/g, preferably about 0.6 dl/g to about 2 dl/g.

120. The arm of any one of claims 115-119, wherein the release rate-modulating film further comprises PEG.

121. The arm of claim 120, wherein the PEG comprises PEG of average molecular weight between about 800 and about 1,200.

122. The arm of claim 120 or claim 121, wherein the PDL-PCL copolymer comprises about 75% to about 95% of the release rate modulating film by weight and the PEG comprises about 5% to about 25% of the release rate modulating film by weight.

123. The arm of claim 120 or claim 121, wherein the PDL-PCL copolymer comprises about 90% of the release rate modulating film by weight and the PEG comprises about 10% of the release rate modulating film by weight.

124. The arm of claim 115, wherein:
(a) PDL comprises about 25% of the PDL-PCL copolymer; or
(b) PDL comprises about 80% of the PDL-PCL copolymer.

125. The arm of any one of claims 115-124, wherein the release rate-modulating film is substantially free of porogen.

126. The arm of any one of claims 115-125, wherein the increase in the weight of the arm due to addition of the release rate-modulating film is about 2% to about 6% of the weight of the uncoated arm.

127. The arm of any one of claims 115-126, wherein the release rate of agent from the arm in aqueous media is substantially linear over at least a 96-hour period.

128. The arm of any one of claims 115-127, wherein the release rate of agent from the arm is substantially the same before and after thermal cycling.

129. A gastric residence system comprising an arm of any one of claims 115-128.

130. A gastric residence system comprising:
one or more arms of any one of claims 115-129; and
a central elastic polymeric component;
wherein the one or more arms are each connected to the central elastic polymeric component via a separate linker component;
wherein the gastric residence system is configured to be folded and physically constrained during administration and is configured to assume an open retention shape upon removal of a constraint;
wherein change between the folded shape and the open retention shape is mediated by the elastic polymeric component that undergoes elastic deformation when the residence system is in the folded shape and recoils when the gastric residence system assumes the open retention shape; and
wherein said linker degrades, dissolves, disassociates, or mechanically weakens in a gastric environment which results in loss of retention shape integrity and passage out of a gastric cavity.

131. The arm of any one of claims 115-123, wherein the release rate-modulating film further comprises a polyethylene glycol-polypropylene glycol-polyethylene glycol (PEG-PPG-PEG) block copolymer.

132. The arm of claim 131, wherein the PEG-PPG-PEG block copolymer comprises PEG-PPG-PEG block copolymer of Mₙ about 14,000 to about 15,000.

133. The arm of claim 131 or claim 132, wherein the PEG-PPG-PEG block copolymer comprises about 75% to about 90% ethylene glycol.

134. The arm of any one of claims 131-133, wherein the (PDL-PCL copolymer):(PEG-PPG-PEG block copolymer) ratio is between about 85:15 to about 95:5 (w/w).

135. The arm of any one of claims 131-133, wherein the (PDL-PCL copolymer):(PEG-PPG-PEG block copolymer) ratio is about 9:1 (w/w).

136. The arm of claim 131-135, wherein:
(a) PDL comprises about 25% of the PDL-PCL copolymer; or
(b) PDL comprises about 80% to about 90% of the PDL-PCL copolymer.

137. The arm of any one of claims 131-136, wherein the release rate-modulating film is substantially free of porogen.

138. The arm of any one of claims 131-137, wherein the increase in the weight of the arm due to addition of the release rate-modulating film is about 2% to about 6% of the weight of the uncoated arm.

139. The arm of any one of claims 131-138, wherein the release rate of agent from the arm in aqueous media is substantially linear over at least a 96-hour period.

140. The arm of any one of claims 131-139, wherein the release rate of agent from the arm is substantially the same before and after thermal cycling.

141. A gastric residence system comprising an arm of any one of claims 131-140.

142. A gastric residence system comprising:
one or more arms of any one of claims 131-140; and
a central elastic polymeric component;
wherein the one or more arms are each connected to the central elastic polymeric component via a separate linker component;
wherein the gastric residence system is configured to be folded and physically constrained during administration and is configured to assume an open retention shape upon removal of a constraint;
wherein change between the folded shape and the open retention shape is mediated by the elastic polymeric component that undergoes elastic deformation when the residence system is in the folded shape and recoils when the gastric residence system assumes the open retention shape; and
wherein said linker degrades, dissolves, disassociates, or mechanically weakens in a gastric environment which results in loss of retention shape integrity and passage out of a gastric cavity.

143. The arm or gastric residence system of any one of claims 1-142, wherein the release rate-modulating film is applied by pan coating.

144. The arm or gastric residence system of any one of claims 1-142, wherein the release rate-modulating film is applied by dip coating.

145. The arm or gastric residence system of any one of claims 1-144, wherein the at least one agent or a pharmaceutically acceptable salt thereof comprises one or more of drug, a pro-drug, a biologic, a statin, rosuvastatin, a nonsteroidal anti-inflammatory drug (NSAID), meloxicam, a selective serotonin reuptake inhibitor (SSRs), escitalopram, citalopram, a blood thinner, clopidogrel, a steroid, prednisone, an antipsychotic, aripiprazole, risperidone, an analgesic, buprenorphine, an opioid antagonist, naloxone, an anti-asthmatic, montelukast, an anti-dementia drug, memantine, a cardiac glycoside, digoxin, an alpha blocker, tamsulosin, a cholesterol absorption inhibitor, ezetimibe, an anti-gout treatment, colchicine, an antihistamine, loratadine, cetirizine, an opioid, loperamide, a proton-pump inhibitor, omeprazole, an antiviral agent, entecavir, an antibiotic, doxycycline, ciprofloxacin, azithromycin, an anti-malarial agent, levothyroxine, a substance abuse treatment, methadone, varenicline, a contraceptive, a stimulant, caffeine, a nutrient, folic acid, calcium, iodine, iron, zinc, thiamine, niacin, vitamin C, vitamin D, biotin, a plant extract, a phytohormone, a vitamin, a mineral, a protein, a polypeptide, a polynucleotide, a hormone, an anti-inflammatory drug, an antipyretic, an antidepressant, an antiepileptic, an antipsychotic agent, a neuroprotective agent, an anti-proliferative, an anti-cancer agent, an antimigraine drug, a prostaglandin, an antimicrobial, an antifungals, an antiparasitic, an anti-muscarinic, an anxiolytic, a bacteriostatic, an immunosuppressant agent, a sedative, a hypnotic, a bronchodilator, a cardiovascular drug, an anesthetic, an anti-coagulant, an enzyme inhibitor, a corticosteroid, a dopaminergic, an electrolyte, a gastro-intestinal drug, a muscle relaxant, a parasympathomimetic, an anorectic, an anti-narcoleptics, quinine, lumefantrine, chloroquine, amodiaquine, pyrimethamine, proguanil, chlorproguanil-dapsone, a sulfonamide, sulfadoxine, sulfamethoxypyridazine, mefloquine, atovaquone, primaquine, halofantrine, doxycycline, clindamycin, artemisinin, an artemisinin derivative, artemether, dihydroartemisinin, arteether, or artesunate.

146. The arm or gastric residence system of any one of claims 1-144, wherein the at least one agent or a pharmaceutically acceptable salt thereof comprises memantine.

147. The arm or gastric residence system of any one of claims 1-144, wherein the at least one agent or a pharmaceutically acceptable salt thereof comprises donepezil.

148. The arm or gastric residence system of any one of claims 1-144, wherein the at least one agent or a pharmaceutically acceptable salt thereof comprises memantine and donepezil.

149. The arm or gastric residence system of any one of claims 1-144, wherein the at least one agent or a pharmaceutically acceptable salt thereof comprises risperidone.

150. The arm or gastric residence system of any one of claims 1-144, wherein the at least one agent or a pharmaceutically acceptable salt thereof comprises dapagliflozin.
